(19) United States
(12) Patent Application Publication
(10) Pub. No.: US 2026/0240865 A1
(43) Pub. Date: Aug. 20, 2026

(54) HARNESSING IKZF:BETA-CATENIN COMPLEXES IN THE TREATMENT OF LYMPHOCYTE ASSOCIATED DISEASES OR CONDITIONS

(71) Applicant: Yale University, New Haven, CT (US)

(72) Inventor: Markus Müschen, Branford, CT (US)

(73) Assignee: Yale University, New Haven, CT (US)

(21) Appl. No.: 18/869,754

(22) PCT Filed: May 24, 2023
(86) PCT No.: PCT/US2023/067398

§ 371 Date: Nov. 26, 2024

Related U.S. Application Data
(60) Provisional application No. 63/346,407, filed on May 27, 2022.

(51) Int. Cl. A61K 31/5517; A61K 31/407; A61K 31/433; A61K 31/475; A61K 31/506; A61K 31/52; A61K 31/573; A61K 31/664; A61K 31/704; A61P 19/02; A61P 35/00; A61P 37/06

(52) U.S. Cl. CPC A61K 31/5517; A61K 31/407; A61K 31/433; A61K 31/475; A61K 31/506; A61K 31/52; A61K 31/573; A61K 31/664; A61K 31/704; A61P 19/02; A61P 35/00; A61P 37/06

(57) ABSTRACT

The present invention relates of treating a lymphocyte associated disease or condition, the method comprising administering to a subject in need thereof an effective amount of an agonist or activator of a β-catenin:Ikaros zinc finger (IKZF) protein complex. The present invention relates also to methods of eradicating pathogenic lymphocyte populations or enhancing adoptive cellular therapy (ACT) (e.g., via preconditioning) in a subject, the method comprising administering to a subject in need thereof a therapeutically effective amount of an agonist or activator of a β-catenin:IKZF protein complex. The present invention also relates to methods of treating a lymphopenic associated disease or condition or enhancing ACT, the method comprising administering to a subject in need thereof an effective amount of an agent that inhibits the expression or function of β-catenin or a β-catenin:IKZF protein complex.

### CROSS-REFERENCE TO RELATED APPLICATIONS

This patent application claims priority to U.S. Provisional Application No. 63/346,407, filed May 27, 2022, the disclosure of which is herein incorporated by reference in its entirety.

### SEQUENCE LISTING

The instant application contains a Sequence Listing which has been submitted electronically in XML file format and is hereby incorporated by reference in its entirety. Said XML copy, created on May 23, 2023, is named 251609_000087_SL.xml and is 20,178 bytes in size.

### FIELD OF THE INVENTION

The present invention relates to methods of treating a lymphocyte associated disease or condition, the method comprising administering to a subject in need thereof an effective amount of an agonist or activator of a β-catenin:Ikaros zinc finger (IKZF) protein complex.

The present invention also relates to methods of eradicating pathogenic lymphocyte populations, the method comprising administering to a subject in need thereof a therapeutically effective amount of an agonist or activator of a β-catenin:IKZF protein complex and to methods of enhancing adoptive cellular therapy (ACT) (e.g., via preconditioning) in a subject, the method comprising administering to a subject in need thereof a therapeutically effective amount of an agonist or activator of a β-catenin:IKZF protein complex. The present invention also relates to methods of treating a lymphopenic associated disease or condition or enhancing ACT, the method comprising administering to a subject in need thereof an effective amount of an agent that inhibits the expression or function of β-catenin or a β-catenin:IKZF protein complex.

### BACKGROUND

Lymphoid malignancies together represent the most frequent type of cancer in children and young adults. Despite steady improvements in clinical outcomes over the past decades, roughly 25% of children who experience bone marrow relapse still exhibit a poor prognosis. In addition, current algorithms of risk-stratification, unfortunately, are unable to distinguish patients that will relapse from those who will respond well to standard-chemotherapy. As a consequence, many patients who would benefit from milder forms of chemotherapy are nonetheless treated with aggressive regimen and will suffer late effects from unnecessary toxicity. For example, among ~110,000 childhood B-ALL survivors in the U.S., main late effects include developmental delays, damage to heart and bone formation, impaired fertility, and secondary cancers. Survivorship of childhood ALL is steadily increasing, thus, highlighting the importance of efforts to reduce toxicity and minimize late effects.

Thus, there exists a need to for effective therapy against lymphoid malignancies and other lymphocyte associated diseases or conditions (e.g., autoimmune diseases, graft versus host disease, etc.). This need can be met with methods and compositions that induce or accelerate the formation of β-catenin: IKZF protein complexes in lymphocytes.

### SUMMARY OF THE INVENTION

In certain aspects, the present disclosure provides a method of treating a lymphocyte associated disease or condition, the method comprising administering to a subject in need thereof an effective amount of an agonist or activator of a β-catenin:Ikaros zinc finger (IKZF) protein complex.

In certain aspects, the present disclosure provides a method of eradicating pathogenic lymphocyte populations, the method comprising administering to a subject in need thereof a therapeutically effective amount of an agonist or activator of a β-catenin:IKZF protein complex.

In certain aspects, the present disclosure provides a method of enhancing adoptive cellular therapy (ACT) (e.g., via preconditioning) in a subject, the method comprising administering to a subject in need thereof a therapeutically effective amount of an agonist or activator of a β-catenin: IKZF protein complex. In some embodiments, the agonist or activator of a β-catenin:Ikaros zinc finger (IKZF) protein complex is administered prior to administering the ACT.

In some embodiments, the agonist or activator of the β-catenin:IKZF protein complex is an agent that inhibits the expression or function of Glycogen Synthase Kinase 3β (GSK3β), Axis Inhibition Protein 1 (AXIN1), Axis Inhibition Protein 2 (AXIN2), Adenomatous Polyposis Coli (APC), and/or beta-transducin repeat containing (beta-TCRP).

In some embodiments, the agonist or activator of the β-catenin:IKZF protein complex is a small molecule, a peptide, an antibody or antibody fragment, an siRNA, an shRNA, a gapmer, an antisense oligonucleotide, an aptamer, PROTAC-degrader, or a site-specific nuclease. In some embodiments, the site-specific nuclease is an engineered homing endo-nuclease or meganuclease, a zinc-finger nucleases (ZFNs), a transcription activator-like effector nucleases (TALENs), or a clustered regularly interspaced short palindromic repeat (CRISPR) system.

In some embodiments, the agent that inhibits the expression or function of GSK3β is a GSK3β inhibitor.

In some embodiments, the GSK3β inhibitor is a small molecule, a peptide, an antibody or antibody fragment, an siRNA, an shRNA, a gapmer, an antisense oligonucleotide, an aptamer, PROTAC-degrader, or a site-specific nuclease.

In some embodiments, the small molecule GSK3β inhibitor is a diazepinoindole, a biindole, an aminopyrimidine, a thiadiazolidine, or a maleimide-based molecule. In some embodiments, the diazepinoindole is LY2090314, the biindole is 6-Bromoindirubin-3′-oxime, the aminopyrimidine is CHIR98014 or CHIR99021, the thiadiazolidine is Tideglusib, or the maleimide-based molecule is 9-ING-41.

In some embodiments, the GSK3β inhibitor is administered to the subject a dose sufficient to result in a steady state plasma concentration of the inhibitor from about 5 nM to about 500 nM. In some embodiments, the inhibitor inhibits GSK3β with an IC₅₀ of 100 nM or less.

In some embodiments, the IKZF protein is IKZF1, IKZF2, or IKZF3. In some embodiments, the IKZF protein is IKZF1 or IKZF3.

In some embodiments, the lymphocyte associated disease or condition is a B-lymphoid malignancy, a T-lymphoid malignancy, or a combination of both. In some embodiments, the lymphocyte associated disease or condition is a premalignant condition or a cancer.

In some embodiments, the premalignant condition is lymphoid clonal hematopoiesis of indeterminate potential (L-CHIP), Monoclonal B lymphocytosis (MBL), or a monoclonal gammopathy of unknown significance (MGUS).

In some embodiments, the cancer is a metastatic cancer. In some embodiments, the cancer is an acute T-lymphoblastic lymphoma/leukemia (T-ALL), a peripheral T-cell lymphoma (PTCL), a cutaneous T-cell lymphomas, an adult T-cell leukemia/lymphoma, an angioimmunoblastic T-cell lymphoma, an extranodal natural killer/T-cell lymphoma, an enteropathy-associated intestinal T-cell lymphoma (EATL), an anaplastic large cell lymphoma (ALCL), a peripheral T-cell lymphoma not otherwise specified cancer (PTCL-NOS), a B-cell acute lymphoblastic leukemia (B-ALL), a diffuse large B-cell lymphoma (DLBCL), a follicular lymphoma, a chronic lymphocytic leukemia (CLL)/small lymphocytic lymphoma (SLL), a mantle cell lymphoma (MCL), a marginal zone lymphoma, a Burkitt lymphoma, a lymphoplasmacytic lymphoma (Waldenstrom macroglobulinemia), a hairy cell leukemia, a primary central nervous system (CNS) lymphoma, a primary intraocular lymphoma, or a non-Hodgkin lymphoma (NHL).

In some embodiments, the lymphocyte associated disease or condition is an autoimmune disease. In some embodiments, the autoimmune disease or condition is rheumatoid arthritis, systemic lupus erythematosus, vasculitis, scleroderma, or Sjogren disease.

In some embodiments, the lymphocyte associated disease or condition is a graft versus host disease (GvHD).

In some embodiments, the inhibitor of the β-catenin:IKZF protein complex is administered in combination with at least one other treatment regimen for the lymphocyte associated disease or condition. In some embodiments, the at least one other treatment comprising glucocorticoids; azathioprine; methotrexate; a combination of vincristine, prednisolone, L-asparaginase, daunorubicin (VPLD); a combination of cyclophosphamide, vincristine, Adriamycin, and dexamethasone (hyper-CVAD); a combination of rituximab, cyclophosphamide, doxorubicin, vincristine, and prednisone (R-CHOP); a combination of cyclophosphamide, doxorubicin, vincristine, and prednisone (CHOP); or combinations thereof.

In certain aspects, the present disclosure provides a method of treating a lymphopenic associated disease or condition, the method comprising administering to a subject in need thereof an effective amount of an agent that inhibits the expression or function of β-catenin or a β-catenin:Ikaros zinc finger (IKZF) protein complex.

In certain aspects, the present disclosure provides a method of enhancing adoptive cellular therapy (ACT) in a subject, the method comprising administering to a subject in need thereof or an ACT preparation a therapeutically effective amount of an agent that inhibits the expression or function of β-catenin or a β-catenin:IKZF protein complex.

In some embodiments, the agent that inhibits the expression or function of β-catenin inhibitor or a β-catenin:Ikaros zinc finger (IKZF) protein complex is a small molecule, a peptide, an antibody or antibody fragment, an siRNA, an shRNA, a gapmer, an antisense oligonucleotide, an aptamer, PROTAC-degrader, or a site-specific nuclease. In some embodiments, the site-specific nuclease is an engineered homing endo-nuclease or meganuclease, a zinc-finger nucleases (ZFNs), a transcription activator-like effector nucleases (TALENs), or a clustered regularly interspaced short palindromic repeat (CRISPR) system. In some embodiments, the β-catenin gene is knocked out or knocked down.

In some embodiments, the lymphopenic associated disease or condition is lymphocytopenia and/or bone marrow failure.

In some embodiments, lymphopenic associated disease or condition is caused by myeloid skewing, immunosenescence, side effects of drug-treatment, bone marrow transplantation, viral infections, and/or immunodeficiencies.

In some embodiments, wherein the disease or condition is a drug-resistant disease or condition.

In some embodiments, the agonist, activator, inhibitor, or agent is administered intravenously, subcutaneously, or orally.

In some embodiments, the agonist, activator, inhibitor, or agent is administered in a dosage range from 5 nM to 100 nM.

### BRIEF DESCRIPTION OF THE DRAWINGS

FIGS. 1A-IG show that B-lymphoid cells are exempt from β-catenin signaling. FIG. 1A: Computational analyses of positive or negative selection of known driver mutations along eight signaling pathways were performed for 14 cancer types, including B-ALL and β-cell lymphoma. FIG. 1B: Frequencies of pathogenic mutations (FATHMM score >0.5) of β-catenin (CTNNB1; filtered for hot spot mutations in exon 3), APC, AXIN1, AXIN2 and GSK3β (GSK3β) are depicted for 14 types of cancer including B-cell malignancies and solid tumors. FIG. 1C: Analysis of Wnt/β-catenin activity in B-cells (CD19⁺ B220⁺), T-cells (CD3⁺) and NK-cells (NK.K1⁺) in Axin2-mTurquoise reporter transgenic mice. FIG. 1D: Transcriptional analysis of 1,389 cancer cell lines by RNA-seq for the expression of CTNNB1 (left). Protein levels of β-catenin were assessed by RPPA (middle) and mass-spectrometry (right) in B-cell malignancies compared to solid tumors. FIG. 1E: CTNNB1 dependency among human cancer cell lines evaluated by CRISPR loss-of function screen. FIG. 1F: Representative immunohistochemical stainings for β-catenin in normal epithelial and lymphoid tissues in comparison to lung cancer (n=15), colon cancer (n=25), malignant melanoma (n=5), mantle cell lymphoma (MCL; n=26), follicular lymphoma (n=38), diffuse large B-cell lymphoma (DLBCL; n=35) and Hodgkin's lymphoma (HD; n=44) using H&E as counterstain. FIG. 1G: Western blot analysis for β-catenin, β-tubulin, and TATA box binding protein (TBP) on nuclear fractions of lung and colon cancer, malignant melanoma, B cell acute lymphoblastic leukemia (B-ALL), DLBCL, MCL, Burkitt's lymphoma, HD and multiple myeloma cell lines. Western blots of the cytoplasmic fractions from the same cell lysates are shown in FIG. 9.

FIGS. 2A-2M show that genetic accumulation of β-catenin suppresses B-cell development and malignant transformation in vivo. FIGS. 2A-B: B-cell development in the bone marrow and spleen of Mb1^{Cre/+} Ctnnb1^{ex3fl} mice was analyzed by flow cytometry. FIG. 2A: The numbers of pro-B cells (CD43⁺ B220^{low} IgM⁻ BP1⁻) and pre-BI cells (CD43⁺ B220^{low} IgM⁻ BP1⁺), pre-BII cells (CD43⁻ B220^{low} IgM⁻), immature B cells (CD43⁻ B220^{low} IgM⁺) and mature B cells (CD43⁻ B220^{high} IgM⁺) in the bone marrow ofMblcre/+ Ctnnble^{x3fl} mice are shown from 6 independent experiments. FIG. 2B: Absolute numbers and frequencies of B220⁺ splenic B-cells and representative FACS plots are shown. Ctnnb1^{ex3fl/+} BCR-ABL1 or NRAS^{G12D} transformed B-ALL cells were transduced with vectors expressing GFP and 4-hydroxy-tamoxifen (4-OHT)-inducible Cre (Cre-ER^{T2}) or ER^{T2}. FIG. 2C: Changes of percentages of GFP⁺ cells were monitored for 8 days following 4-OHT addition, data representative of three independent experiments (triplicates). FIG. 2D: B-ALL cells were sorted for GFP expression and plated for colony formation assays after 4-OHT treatment. Representative images for 10 days after plating. (FIG. 2E: Cell cycle phases of Ctnnb1^{ex3fl/+} NRAS^{G12D} or BCR-ABL1 B-ALL cells were measured by EdU incorporation in combination with DAPI staining 2 days after β-catenin accumulation. Data shown are representative of two independent experiments (triplicates). FIGS. 2F-G: Extreme limiting dilution analysis (ELDA) was performed to assess effects of β-catenin accumulation on leukemia-initiation capacity (LIC) of BCR-ABL1-driven B-ALL cells. FIG. 2F: Kaplan-Meier analysis of overall survival in each group and dose level (n=4; P=8.2E-10; Log-rank test). FIG. 2G: LIC was determined in B-ALL cells with β-catenin accumulation (1 in 40,063 cells) and control cells (1 in 1,042; P=0.0006). FIGS. 2H-I: Gene expression changes were studied by RNA-seq analysis in Ctnnb1^{x3fl/+} B-ALL one day after 4-OHT-treatment (n=4). FIG. 2H: Gene set enrichment analysis (GSEA) identified depletion of Myc target genes and enrichment of Ikaros target genes as top-ranking gene sets following β-catenin accumulation. FIG. 2AI: Genes that were upregulated (n=354) or down-regulated (n=119) upon β-catenin stabilization are shown as heatmap. FIG. 2J: Flow cytometry analysis to validate CD5, Ccr2 and CD244 (2B4) upregulation 3 days after Cre-mediated stabilization of β-catenin. FIG. 2K: Changes in protein levels of β-catenin, Myc, Dgka, Prdm1 were studied by Western blot 0-3 days after β-catenin activation. BCR-ABL1 transformed Ctnnb1^{ex3fl/+} B-ALL cells expressing Cre-ER^{T2} or ER^{T2} (puromycin selected) were transduced with GFP-tagged Myc or empty vector (EV). FIG. 2L: Expression of β-catenin and Myc in FACS-sorted GFP⁺ cells was confirmed by Western blot 3 days after 4-OHT treatment. FACS analyses were performed to monitor enrichment or depletion of GFP⁺ cells (Myc vs EV) upon β-catenin activation. Representative data from three independent experiments (triplicates) is shown. FIG. 2M: Colony formation ability of cells expressing Myc, or empty vector (EV) was assessed 2 days after 4-OHT induced β-catenin accumulation. Data shown is a representative of two independent experiments (triplicates).

FIGS. 3A-3F show deleterious effects of β-catenin-accumulation in B-lymphoid but not myeloid and epithelial cells. Human cancer cell lines or patient derived xenografts were transduced with Tet-3G doxycycline-inducible vectors for expression of GFP tagged stabilized β-catenin with point mutations of GSK3β-phosphorylation sites (CTNNB1) or empty vector (EV). FIGS. 3A-3B: Doxycycline was added to induce expression of β-catenin and GFP and changes in the percentages of GFP⁺ cells were monitored by FACS. Data shown are representative of two independent experiments (triplicates). FIG. 3C: B-ALL (BLQ5), mantle cell lymphoma (JEKO1), and colon cancer (LOVO) cell lines carrying inducible β-catenin constructs were treated with doxycycline for two days. Western blot was performed to detect the expression of β-catenin, MYC and the lymphoid transcription factors IKZF1 and IKZF3, using R-actin as loading control. FIG. 3D: Cell viability following β-catenin accumulation was monitored over time by flow cytometry based on Annexin V and DAPI staining. Data shown are representative of two independent experiments. FIG. 3E: One day after doxycycline treatment GFP⁺ cells were FACS sorted (99.8% pure) and plated on methylcellulose medium for colony forming assays. Colonies were imaged and counted 14 days after plating. Representative images from two independent experiments are shown (triplicates). Cell lines left to right: BV173 (B-ALL), JEKO and Z138 (mantle cell lymphoma), and MV4-11 (acute myeloid leukemia). FIG. 3F: Cell cycle analyses were performed by measuring EdU incorporation 2 days after doxycycline mediated expression of stabilized β-catenin. Changes in frequencies of cells in S phase following β-catenin accumulation were shown. Data are representative of two independent experiments (two replicates each). Cell lines left to right: BV173 (B-ALL), JEKO (mantle cell lymphoma), MV4-11 (acute myeloid leukemia) and SW480 (colon cancer).

FIGS. 4A-4H show that β-catenin forms repressive complexes with B-lymphoid transcription factors Ikzf1 and Ikzf3. FIGS. 4A-4B: Proteins bound to β-catenin in B-ALL cells from Ctnnb1^{ex3fl/+} mice were enriched by co-IP, identified by mass spectrometry, and plotted based on statistical significance and log 2-fold enrichment over IgG background control (n=4). Proteins with the most prominent binding to β-catenin included Ikaros factors Ikzf1 and Ikzf3 and members of the repressive NuRD complex Chd4, Gatad2a, Gatad2b, Mta1, Mta2, Mdb3, Rbbp4, Hdac1, Hdac2. FIG. 4B: β-catenin interacting proteins were validated by co-IP and Western blot in whole cell lysates (Input), proteins bound (Elute) and flow-through (FT) to isotype control or antibodies against β-catenin, using Stat5 as negative control. Co-IP experiments with antibodies against β-catenin or control Ig were performed in human B-ALL (MXP2), B-cell lymphoma (JEKO), AML (MOLM13), colon (SW480) and lung (H446) cancer cell lines expressing doxycycline inducible β-catenin. Eluted proteins were analyzed by mass-spectrometry. FIG. 4C: Principal component analysis was performed to cluster cell lines based on similarity of β-catenin interactomes. FIG. 4D: Heatmap of proteins that were enriched for β-catenin binding relative to Ig-control in B-ALL, mantle cell lymphoma (MCL), myeloid leukemia (AML), colon and lung cancer cell lines. FIG. 4E: Whole cell lysates (Input), proteins bound and flow-through (FT) with β-catenin-antibodies or control Ig were analyzed by Western blotting to study interactions between β-catenin and Ikaros factors (IKZF1, IKZF3), NuRD complex components (MTA1, MTA2, GATAD2A) and TCF7L2, and LEF1 in B-ALL (PDX2), myeloid leukemia (JURL-MK1) and colon cancer (SW620) cells 16 hours following pharmacological β-catenin stabilization (LY2090314, 20 nM). FIG. 4F: β-catenin binding proteins in each cell type were plotted as a function of background binding (x-axis, non-specific binding defined by CRAPOME database) and log 2-fold enrichment over control Ig (y-axis). FIG. 4G: Changes in β-catenin interactomes in B-ALL cells upon Ikaros factor deletion (gIkzf1/3) were analyzed by co-IP and mass-spectrometry. Proteins bound to β-catenin were plotted based on significance (y-axis) and log 2-fold enrichment (x-axis) compared to B-ALL cells without deletion of Ikaros factors (gNT; n=3). FIG. 4H: Amplification of Ikaros-mediated gene expression changes by β-catenin: depletion of genes repressed by Ikaros factors and enrichment of genes indirectly activated by Ikaros factors in murine B-ALL cells upon β-catenin accumulation.

FIGS. 5A-5J show that β-catenin functions as an amplifier of Ikaros-mediated gene expression changes. BCR-ABL1-transformed Ctnnb1^{ex3fl/+} B-ALL cells were gene-edited with crRNAs targeting Ikaros factors (Ikzf1, Ikzf3) individually or both or non-targeting crRNAs (gNT). Deletion of Ikaros factors was confirmed by Western blot in clonal cell lines established from single cells. Multiple clones were studied for each genotype. Ctnnb1^{ex3fl/+} B-ALL cells were transduced with 4-OHT-inducible GFP-tagged Cre-ER^{T2} or ER^{T2}. Color code for boxes in FIGS. 5A-5E: light grey box (β-catenin baseline), dark grey box with an X (β-catenin accumulated), medium grey box (Ikaros factors baseline) and white box (Ikaros factors deleted). FIG. 5A: Western blot was performed for β-catenin, Ikzf1, Ikzf3, Myc and β-actin two days after induction of Cre and β-catenin accumulation. FIG. 5B: Competitive fitness of B-ALL clones was assessed in the presence or absence of β-catenin accumulation and deletion of either Ikzf1, Ikzf3 or both Ikaros factors, using non-targeting crRNAs (gNT) as reference. FIG. 5C: Heatmap to show changes in Myc target gene expression levels upon β-catenin activation with and without concurrent deletion of both Ikaros factors (Ikzf1, Ikzf3). FIG. 5D: Western blot analyses to measure protein levels of β-catenin, Myc, Ikzf1 and Ikzf3 in relation to β-actin for 0-3 days after 4-OHT addition. FIG. 5E: Colony forming assays for B-ALL cells with and without Ikaros factor deletion and with and without 3-catenin accumulation (2 days) are shown. Representative images and colony numbers from three independent experiments are shown at 10 days after plating (triplicates). FIG. 5F: GSEA plots for enrichment of β-catenin signaling (left) and MYC target genes (right) upon β-catenin accumulation and in the presence (bottom) or absence (top) of Ikaros factor deletion. FIG. 5G: Quantification of changes in H3K27Ac ChIP-seq signals at β-catenin target regions vs. other regions following β-catenin accumulation in the presence or absence of Ikaros factor deletion. FIG. 5H: ChIP-qPCR to measure enhancer activity (H3K27ac) and recruitment of NuRD complex components (MTA2 and CHD4) to the Myc superenhancer region (BENC-C) in B-ALL cells upon deletion of β-catenin (white circles) or accumulation of β-catenin (dark grey circles) in comparison to wild type cells (light grey circles). Data were pooled from 7 independent qChIP experiments. FIG. 5I: Murine B-ALL cells with and without engineered deletion of β-catenin were plated on methylcellulose. Primary (1^{st}) and secondary (2^{nd}) platings are shown, representative images and average counts of primary and secondary colonies from three independent experiments. FIG. 5J: Murine B-ALL cells with (white box) and without engineered deletion of β-catenin (light grey box) were transduced with vectors for inducible expression of GFP-tagged IKZF1 (dark grey box with an X) or GFP empty vector (medium grey box). Changes in the frequencies of GFP⁺ cells were monitored by flow cytometry. Representative data from three independent experiments are shown.

FIGS. 6A-6J show that mutation of a single Ikaros-motif of the BENC-C region subverts β-catenin-mediated repression of MYC. FIG. 6A: ChIP-qPCR analysis of recruitment of NuRD complex components (MTA2 and CHD4) to the BENC-C enhancer region in Ctnnb1^{ex3fl/+} B-ALL cells. Genotypes are denoted by light grey boxes (β-catenin baseline), dark grey with an X boxes (β-catenin accumulation), medium grey boxes (Ikaros factors baseline) and empty boxes (Ikaros-null or β-catenin-null). Data represent a pool of 6 independent experiments. FIG. 6B: Quantification of H3K27ac ChIP-seq signals at BENC enhancer regions, other regions with binding of both Ikaros factors and β-catenin (Co-bound) and all other regions. H3K27ac ChIP was performed with and without β-catenin accumulation and in the presence or absence of Ikaros factor deletion. FIG. 6C: BENC elements C and D were analyzed for changes in β-catenin, Ikzf1, and Ikzf3 binding and H3K27 acetylation, upon induction of β-catenin in B-ALL cells with and without deletion of Ikaros factors. FIG. 6D: Identification of Ikaros binding motifs in the BENC-C (m1, m2) and BENC-D (m3) elements. FIGS. 6E-6J: Homology directed repair (HDR)-mediated editing of the BENC-C m1 motif to generate a new EcoRI site. To abrogate the binding of Ikzf1 and Ikzf3, the Ikaros core motif GGGAA was mutated, and clonal cell lines were generated and analyzed by (FIG. 6E) Sanger sequencing and (FIG. 6F) EcoRI digestion and gel electroporation. FIGS. 6G-6H: Western blot analysis of Myc protein levels one day after β-catenin accumulation in B-ALL cells carrying intact or mutated BENC-C Ikaros m1 motifs. FIGS. 6I-6J: Growth kinetics of B-ALL cells with intact and mutant BENC-C Ikaros m1 motif following Cre-mediated induction of β-catenin. FIG. 6I: Representative FACS plots and (FIG. 6J) changes in the percentages of GFP⁺ cells are depicted.

FIGS. 7A-7L show Pharmacological engagement of β-catenin-Ikaros complexes for targeted repression of MYC. FIG. 7A: B-ALL (MXP2, LAX2, BLQ5, IAH8R), mantle cell lymphoma (MCL; JEKO1), colon (SW480, LOVO, HT-29), and lung cancer (H82, H446) cell lines were treated with the GSK3β small molecule inhibitor LY2090314 (20 nM) for one day. B-catenin, MYC and IKZF1 protein levels were assessed by Western blot, using 3-actin as loading control. FIG. 7B: Human B-ALL cells (BV173) were edited with crRNAs targeting β-catenin (gCTNNB1) or non-targeting crRNAs (gNT) and single cell-derived colonies were generated (FIG. 16B). B-ALL cells with (clones 1E4, 1C3; gCTNNB1) or without (clones 2C2, 2D9; gNT) deletion of β-catenin were treated with LY2090314 for 3 days at the indicated concentrations and relative viability was determined by luminescence measurements. FIG. 7C: Human B-ALL cells (BV173) with and without deletion of CTNNB1 were treated with LY2090314 (20 nM) for 16 hours to force accumulation of β-catenin. Western blot was performed to analyze β-catenin and MYC levels. FIG. 7D: Growth inhibition by the GSK3β inhibitor LY2090314 was compared for human B-ALL samples from patients who responded to conventional chemotherapy (sensitive) and from patients with refractory B-ALL (refractory). FIG. 7E: Sensitivity to LY2090314 was assessed in a panel of 28 B-ALL, B-cell lymphoma, myeloid leukemia, colon, and lung cancer cell lines. Growth inhibitory effects were shown as heatmap. FIG. 7F: B-ALL cells, myeloid leukemia and colon cancer cell lines were treated with LY2090314 at concentrations between 0 up to 200 nM for 3 days and cell viability was determined by normalizing the luminescence signal of treated cells to untreated cells. FIG. 7G: Responses to LY2090314 in 343 epithelial cancer cell lines (Prism Drug Repurposing Secondary Screen)⁴⁶ and 17 B-lymphoid cell lines (B-ALL, 7 B-cell lymphoma; red circles) were plotted as IC₅₀ values (nM). FIG. 7H: Drug responses (AUC) to the GSK3β-inhibitor CHIR99021 were plotted for 84 B-cell lymphomas with (8q24+; n=31) and without (8q24-; n=53) MYC rearrangement. FIG. 7I: Computational analyses of gene expression (biomarker) correlations with responses to the GSK3β-inhibitor CHIR99021 in epithelial cancers and B-lymphoid cell lines⁴⁶. Expression of Ikaros-factors was positively associated with sensitivity to CHIR99021, while expression of β-catenin and the epithelial marker TEAD1 correlate with CHIR99021-resistance. FIGS. 7J-7L: Luciferase-labelled LAX2 cells were injected into sub-lethally irradiated NSG mice. Mice were either treated with 10 mg/kg LY2090314 or vehicle control. FIG. 7J: Leukemia burden was assessed by bioluminescence imaging at day 18 (top), 28 (middle) and 42 (bottom) following transplantation. FIG. 7K: Kaplan-Meier analysis of overall survival in each group (n=9, P=6.5E-05; calculated by Logrank test). FIG. 7L: Effect of LY2090314 on leukemia-initiation was studied by transplanting limiting doses (100-2,500 cells) of B-ALL cells prior to treatment into sub-lethally irradiated NSG mice.

FIGS. 8A-8C show lack of β-catenin expression and activity in B-lymphoid cells. FIG. 8A: Immunohistochemical staining for β-catenin expression in human lymphoid tissues, including bone marrow (n=7), spleen (n=6), lymph node (n=8), tonsil (n=9) as well as epithelial tissues, including colon (n=7), liver (n=9), pancreas (n=12), kidney (n=8), lung (n=8) and skin (n=7). Representative images are shown. β-catenin signaling was measured in B-lymphoid (FIG. 8B) and T-lymphoid (FIG. 8C) cells in mice carrying the Axin2-mTurquoise transgene²⁸ (dark gray) relative to background signal in control mice lacking the reporter transgene (light gray). Data shown are representative of three mice from two independent experiments. FIG. 8B: For B-lymphoid cells, bone marrow B220⁺ CD43⁺ B-cell progenitors were separated as Fraction A (Bp1⁻ CD24⁻), B (Bp1⁻ CD24⁺), C (Bp1^{lo} CD24⁺), C′ (Bp1^{hi} CD24⁺) and B220⁺ CD43⁻ B-cells were classified as Fraction D (IgM⁻ IgD⁻), E (IgM⁺ IgD⁻) and F (IgM⁺ IgD⁺). Among B220⁺ splenic B-cells, immature (Immature, CD21⁻ CD23⁻), marginal zone (MZ, CD21⁺ CD23⁻) and follicular (CD21⁺ CD23⁺) B-cells were studied. FIG. 8C: β-catenin signaling was measured in CD4⁻ CD8⁻ double negative (DN), CD4⁺ CD8⁺ double positive thymocytes as well as CD4⁺ and CD8⁺ single positive T-cells. DN thymocytes were further separated as DN1-4 based on CD25 and CD44 expression.

FIGS. 9A-9B show lack of β-catenin expression and activity in B-malignancies. FIG. 9A: β-catenin expression was visualized by immunohistochemistry on tissue microarrays from B-lymphoid malignancies, including mantle cell lymphoma (n=12), follicular lymphoma (n=24), DLBCL (n=24) and Hodgkin's lymphoma (n=24), as well as epithelial cancers, including colon cancer (n=12), lung cancer (n=12) and malignant melanoma (n=3). FIG. 9B: Western blot analysis of β-catenin expression in cytoplasmic fractions of epithelial cancers, including lung and colon cancer, malignant melanoma, as well as B-lymphoid malignancies, including B-ALL, diffuse large B-cell lymphoma (DLBCL), mantle cell lymphoma (MCL), Burkitt's, Hodgkin's disease (HD) and multiple myeloma cell lines. β-tubulin and TBP were used to indicate purity of cytoplasmic and nuclear fractions, respectively. Western blots of the nuclear fractions from the same cell lysates are shown in FIG. 1G.

FIGS. 10A-10D show that genetic accumulation of β-catenin suppresses B-cell development in vivo. FIG. 10A: Bone marrow pre-B cells from Ctnnb1^{ex3fl/+} mice were transduced with 4-hydroxy-tamoxifen (4-OHT)-inducible Cre-ER^{T2} or ER^{T2} constructs. Upon addition of 4-OHT, activation of Cre leads to excision of GSK3β-phosphorylation sites, preventing GSK3β-mediated degradation of β-catenin. Western blot analysis was performed to visualize β-catenin accumulation at the times indicated following 4-OHT addition. FIG. 10B: Ctnnb1^{ex3fl/+} mice were crossed with Mb1^{Cre/+} for B-cell-specific excision of GSK3β-phosphorylation sites. In vitro differentiation of hematopoietic stem cells from the bone marrow of Mb1^{Cre/+} Ctnnb1^{ex3fl/+} and Mb1^{Cre/+} Ctnnb1^{+/+} control mice into pro-B and pre-B cells was studied in the presence of IL7. The frequencies of B220⁺ B cells were measured by FACS 7-11 days after removal of Flt3L and SCF. Data shown represent a pool of 5 independent experiments. FIGS. 10C-10D: B-cell development in the bone marrow and peripheral lymphoid organs of Mb1^{Cre/+} Ctnnb1^{ex3fl/+} and Mb1^{Cre/+} Ctnnb1^{+/+} mice was studied by flow cytometry. FIG. 10C: Relative fractions (%) of B-cell precursor subsets in the bone marrow of the mice are shown for both genotypes. Bone marrow B-cell precursors were distinguished as pro-B cells (CD43⁺ B220^{low} IgM⁻ BP1⁻), pre-BI cells (CD43⁺ B220^{low} IgM⁻ BP1⁺), pre-BII cells (CD43⁻ B220^{low}, IgM⁻), immature B cells (CD43⁻ B220^{low} IgM⁺) and mature B cells (CD43⁻ B220^{high} IgM⁺). FIG. 10D: Representative FACS plots, absolute numbers and fractions (%) of B-cells in the peripheral lymph nodes of Mb1^{Cre/+} Ctnnb1^{ex3fl/+} and Mb1^{Cre/+} Ctnnb1^{+/+} mice are shown.

FIGS. 11A-11F show B-lymphoid-specific β-catenin-Ikaros factor complexes. FIG. 11A: B-ALL (MXP2), B-cell lymphoma (JEKO), T-ALL (KOPT-K), AML (MOLM13), colon cancer (SW480) and lung cancer (H446) cell lines were transduced with Tet-3G transactivator and Tre-3G for doxycycline-inducible expression of β-catenin. Co-IP experiments with antibodies against β-catenin or control IgG were performed for input cell lysates, β-catenin-binding of Ikaros factors IKZF1, IKZF2, IKZF3 and NuRD complex component MTA2, as well as AXIN1 (positive control), shown by Western blot. FIGS. 11B-11C: B-ALL (PDX2), colon (n=3) and lung (n=3) cancer cell lines were transduced with Tet-3G transactivator and Tre-3G for doxycycline-inducible expression of GFP-tagged IKZF1 or EV. FIG. 11B: B-ALL (PDX2) and colon (SW480, HT-29) cancer cell lines were treated with doxycycline for 2 days to induce IKZF1 expression and 16 hours with the GSK3β small molecule inhibitor LY2090314 to accumulate β-catenin. Western blot was performed to study the MYC levels in relation to IKZF1 and β-catenin expression. FIG. 11C: Expression of GFP-tagged IKZF1 or empty vector were induced by addition of doxycycline. GFP⁺ cells were monitored by flow cytometry. Changes in frequencies of GFP⁺ cells were normalized to controls. Representative data of 2 independent experiments (triplicates). FIGS. 11D-11F: Ctnnb1^{ex3fl/+} B-ALL (BCR-ABL1) cells were transduced with Tet-3G transactivator and Tre3G for doxycycline-inducible expression of the myeloid transcription factor CEBPα or empty vector (EV). B-ALL cells carrying inducible CEBPα were subsequently transduced with GFP-tagged Cre-ER^{T2} or ER^{T2} vectors for excision of GSK3β phosphorylation sites. CEBPα-driven myeloid reprogramming was induced upon addition of doxycycline. FIG. 11D: Flow cytometry analysis was performed to identify myeloid (Mac1⁺) and B-lymphoid (CD19⁺) cells two days after doxycycline treatment. FIG. 11E: Western blot analysis to measure CEBPα, Ikzf1, Ikzf3 and Myc levels following β-catenin accumulation in B-ALL after CEBPα myeloid reprogramming (CEBPα) or EV conditions. FIG. 11F: Changes in frequencies of GFP⁺ cells were monitored by FACS for 6 days after 4-OHT mediated activation of Cre and accumulation of β-catenin. Data shown is a representative of three independent experiments with three replicates each.

FIGS. 12A-12D show interactions between Ikaros factors and β-catenin in transcriptional regulation. FIG. 12A: To assess whether Ikaros factors can only bind to β-catenin as Ikzf1/Ikzf3 heterodimers (i.e. both Ikzf1 and Ikzf3 are required for binding), CRISPR-mediated deletion of Ikzf1 (clone 1C7 Ikzf1^{-/-} Ikzf3^{+/+}) and Ikzf3 (clone 2F9 Ikzf1⁺⁺ Ikzf3^{-/-}) were engineered in Ctnnb1^{ex3fl/+} B-ALL cells carrying 4-OHT-inducible Cre-ER^{T2}. Co-IP for β-catenin was performed in cells with single deletion of Ikzf1 or Ikzf3. Western blot analysis for β-catenin, Ikzf1, Ikzf3 and Axin1 in whole cell lysates (input), proteins bound (elute) and flow through (FT) to antibodies against β-catenin or control (Ig). Binding of Ikzf1 alone and Ikzf3 alone to β-catenin remained intact. FIG. 12B: Gene expression changes induced by β-catenin accumulation in the presence and absence of deletion of both Ikaros factors (Ikzf1/3^{-/-}) are shown as heatmap. Genes that are repressed (left heatmap) or activated (right heatmap) by β-catenin and how gene expression changes were affected by Ikaros-deletion are shown. FIG. 12C: Effects of Ikaros factor-deletion (Ikzf1/3^{-/-}) on expression of 3-catenin target genes (y-axis, log 2 fold change) vs. β-catenin binding (x-axis, log 2 fold change) are shown. FIG. 12D: Correlation of gene expression changes (y-axis, log 2 fold change) with changes in active enhancer regions, H3K27ac signal (x-axis, log 2 fold change) upon loss of Ikzf1 and Ikzf3. Absence of Ikaros-factors allowed de-novo binding of β-catenin (gained) and activation of extra-lineage genes such as Tead1, Tbx19, Lmo1, Lhx2 while lymphoid-specific genes such as Blk1, Bach2, Foxo1 lost binding of β-catenin (lost), H3K27ac and were silenced. Note prominent increased β-catenin binding and increased H3K27ac marks at Myc-BENC superenhancer regions (FIG. 12D) upon Ikaros-deletion, consistent with increased Myc expression (FIG. 12C).

FIGS. 13A-13B show that lenalidomide-induced degradation of Ikaros factors relieves β-catenin-mediated repression of MYC. FIG. 13A: Patient derived B-ALL xenografts (PDX, SFO5) were treated with lenalidomide (0.5 μM) to induce CRBN-CRL4-mediated degradation of IKZF1 and IKZF3 Ikaros factors. SFO5 cells were treated with the GSK3β-inhibitor LY2090314 (20 nM) to accumulate β-catenin. Western blot analysis was performed for β-catenin, IKZF1, IKZF3, MYC and P-actin. FIG. 13B: Human B-ALL xenograft cells (SFO5) were treated with lenalidomide (0.5 μM) or vehicle for 2 days and plated for colony formation experiments. Representative images and normalized counts (setting mean of vehicle controls as 100%) from two independent experiments (triplicates) are shown.

FIGS. 14A-14D show that Ikaros factors profoundly impact β-catenin-binding and β-catenin-mediated gene expression but not vice versa. ChIP-seq analysis was performed for Ikzf1, Ikzf3 and β-catenin in Ctnnb1^{e3f1/+} B-ALL cells upon β-catenin accumulation and deletion of Ikaros factors. FIG. 14A: Venn diagram shows the number of regions only bound by β-catenin (4,356), Ikaros factors only (4,596) or both (11,354). Of 15,710 β-catenin peaks, 11,354 (72.2%) were also bound by Ikaros factors. FIG. 14B: Changes in global distribution of Ikzf1 and Ikzf3 peaks upon β-catenin accumulation. 87% of Ikzf1 and Ikzf3 peaks remained unchanged upon β-catenin accumulation. Color coding for light grey box (β-catenin baseline), dark grey with an X box (β-catenin accumulation) and medium grey box (Ikzf1/3 baseline). FIG. 14C: Effects of β-catenin accumulation on Ikaros-factor binding (top) and effects of Ikaros factor deletion on β-catenin binding (bottom) are shown as dot plots for individual ChIP-seq peaks. For each peak, x-axes denote baseline ChIP-seq signals and y-axes show log 2-fold changes for Ikaros binding upon β-catenin accumulation (top) and β-catenin-binding upon Ikaros deletion (bottom). FIG. 14D: Likewise, effects of β-catenin accumulation (top) or Ikaros factor deletion (bottom) on mRNA levels are shown. Baseline levels for each gene are shown on the x-axes, log 2-fold changes denoted on the y-axis for each target gene.

FIGS. 15A-15C show that Ikaros factors compete with TCF7 family transcription factors for binding to β-catenin. FIG. 15A: ChIP-seq analysis to study genome-wide distribution of β-catenin peaks, colocalization with Ikzf1 and Ikzf3 Ikaros factors as well as H3K4me3 and H3K27ac histone marks. Changes of Ikaros and β-catenin peaks as well as H3K4me3 and H3K27ac histone marks were assessed in the presence and absence of Ikaros deletion (empty boxes) and inducible accumulation of β-catenin (dark grey with an X boxes). Ikzf1 and Ikzf3 deletion enabled binding of β-catenin to inactive enhancers and their subsequent activation (gained H3K27ac). Regions that gained β-catenin binding (n=1,202) are bound by Ikzf1 and Ikzf3 Ikaros factors (P=2.5E-62). Deletion of Ikaros factors resulted in redistribution of β-catenin to canonical Tcf7 (P=1.0E-42), Tcf712 (P=1.0E-17) and Tcf711 (P=1.0E-16) motifs. FIG. 15B: β-catenin interacting proteins were studied in the presence or absence of Ikzf1 and Ikzf3 Ikaros factors by Co-TP. Western blot analysis was performed in whole cell lysates (input), proteins bound (elute) and flow through (FT) after Co-TP with antibodies against β-catenin or control Ig antibodies. Co-TP was performed under conditions of β-catenin accumulation (dark grey with an X box) and in the presence Ikaros factor deletion (empty boxes) or Ikaros baseline levels (light grey boxes). Deletion of Ikaros factors enabled binding of β-catenin to Tcf7 and increased interactions with Tcf712 and Tcf711. FIG. 15C: Scenario of transcription factor complexes with β-catenin in epithelial cells and B-lymphoid cells: β-catenin pairs with TCF7/TCF7L1/TCF7L2 factors for transcriptional activation of Myc at Wnt responsive elements (WRE) and epithelial enhancer regions (top). In B-cells, Ikaros factors outcompete TCF7 to bind to β-catenin. Thereby, Ikaros factors and β-catenin cooperate for effective recruitment of repressive NuRD complexes to lymphoid BENC enhancer regions of Myc, resulting in transcriptional repression of Myc (bottom left). Loss of Ikaros factors (Ikzf1 and Ikzf3) enables interactions between β-catenin and TCF7-family factors to restore transcriptional activation of Myc (bottom right).

FIGS. 16A-16G show that β-catenin enables Ikaros-mediated tumor suppression. Mouse (FIG. 16A) and human (BV173) (FIG. 16B) B-ALL cells were gene-edited with guides targeting Ctnnb1 (gCtnnb1) or non-targeting controls (gNTC). Multiple single-cell clones were selected based on evidence for successful deletion of β-catenin in cells treated with GSK3β-inhibitor LY2090314 to force β-catenin accumulation (Western blot). FIG. 16C: ChIP-qPCR was performed for H3K27ac histone marks, reflecting enhancer activity, and recruitment of NuRD complex components (MTA2 and CHD4) at the Igll1 promoter (Ikaros target gene), the Myc promoter, epithelial Myc enhancer regions as well as the lymphoid BENC Myc enhancer region. For each region, H3K27ac, MTA2 and CHD4 ChIP was performed for B-ALL cells carrying β-catenin deletion (empty circles) or intact β-catenin (light grey circles). Data shown represents a pool of four independent experiments. FIG. 16D: Murine myeloid progenitor cells with deletion of β-catenin (gCtnnb1) or non-targeting control (gNTC) were plated in primary (1^{st}) and secondary replatings (2^{nd}) for colony forming assays. Representative images from primary and secondary colonies are shown. Western blot was performed to validate β-catenin loss (representative of two independent experiments). FIG. 16E: Human AML xenografts were edited with guides targeting CTNNB1 (gCTNNB1) or non-targeting control (gNTC) and serially plated on methylcellulose medium. Representative images from primary (1^{st}) and secondary (2^{nd}) colonies from two independent experiments are shown. β-catenin deletion was validated by Western blot. FIG. 16F: Mouse B-ALL cells with deletion of β-catenin (clone 2A6) or non-targeting control (clone 1C4) were plated on methylcellulose and 7 days later secondary plating was performed. Number of primary (1^{st}) and secondary (2^{nd}) colonies from three independent experiments are shown (Images, FIG. 5I). Cell cycle phases were studied by Edu incorporation and DAPI staining (n=2). FIG. 16G: Human B-ALL xenografts (SFO5) with CTNNB1 deletion (gCTNNB1) or non-targeting control (gNTC) were compared in a serial plating assay. Number of colonies in primary (1^{st}) and secondary (2^{nd}) plating and Western blot for validation of β-catenin-deletion are shown.

FIGS. 17A-17B show that β-catenin and Ikaros factors target the BENC-C superenhancer region of MYC and are both are required for NuRD complex recruitment. FIG. 17A: ChIP-qPCR was performed for NuRD complex components (MTA2 and CHD4) at the Igll1 promoter (positive control as known Ikaros and NuRD complex target gene), the Myc promoter, epithelial Myc enhancer regions as well as lymphoid BENC Myc enhancer regions. For each region, MTA2 and CHD4 ChIP was performed for B-ALL cells with induced β-catenin accumulation (dark grey with an X boxes), β-catenin deletion (empty boxes) or intact β-catenin (light green boxes), as well as deletion of Ikaros factors (empty boxes) or intact Ikaros factors (medium grey boxes). Data shown represent a pool of 6 independent experiments. FIG. 17B: ChIP-seq analysis for β-catenin, Ikaros factors Ikzf1 and Ikzf3, histone marks H3K27ac and H3K4me3 is shown for the Myc locus, including upstream Myc promoter regions and long-range transcriptional enhancers of Myc in B-ALL cells from Ctnnb1^{ex3fl/+} mice. Heat map of H3K27ac distribution marking active enhancer regions, shows that most of the H3K27ac enhancer activity is concentrated in lymphoid blood enhancer cluster (BENC) regions in B-ALL cells (top). Peak density plots show colocalization of β-catenin, Ikzf1 and Ikzf3 peaks and their concentration at the BENC enhancer regions (middle). Close-up view of ChIP-seq peaks of β-catenin, Ikzf1, Ikzf3, H3K27ac at BENC enhancer elements C and D in B-ALL cells with accumulation of 3-catenin (dark grey with an X boxes), β-catenin baseline (light grey boxes), Ikaros factor deletion (empty boxes) or Ikaros baseline (medium grey boxes) is shown (bottom). Ikaros factors and β-catenin show marked enrichment at BENC-C and BENC-D regions. While accumulation of β-catenin depleted H3K27ac marks at BENC-C and -D enhancer regions, 3-catenin had the opposite effect and increased BENC-C enhancer activity and H3K27ac signals when Ikaros factors (Ikzf1 and Ikzf3) were deleted (P=0.002, FIG. 6B).

FIG. 18A-18D show repurposing of clinically approved GSK3β-inhibitors for refractory B-cell malignancies. Responses to GSK3β small molecule inhibitors were assessed in three B-cell leukemia (B-cell) cell lines and each one myeloid, colon and lung cancer cell line. FIG. 18A: Chemical structures of tested compounds are shown. FIG. 18B: Drug responses are shown as a heatmap for LY2090314, 6-bromo-indirubin 3′-oxime (6B10), Tideglusib, 9-ING-41, CHIR98014 and CHIR99021 in B-cell lines (PDX2, BV173, LAX2) vs. other cell lines (THP1, SW620, H82) at the indicated concentrations. FIG. 18C: B-ALL (PDX2) cells were treated with indicated GSK3 inhibitors for 16 hours. Changes in protein levels of β-catenin and Myc in relation to 3-actin levels were shown by Western blot. FIG. 18D: Summary of Phase I and Phase II clinical trials with GSK3β inhibitors for a variety of clinical indications. In a total of 22 clinical trials, all tested small molecule inhibitors achieved favorable safety and PK/PD profiles at micromolar plasma concentrations (Cmax). None of inhibitors achieved clinical responses. Moderate adverse effects included diarrhea, anemia and lymphopenia.

FIGS. 19A-19H show genetic hyperactivation of β-catenin in murine NRAS^{G12D} and BCR-ABL1-driven B-ALL. NRASG^{12D}-driven (FIGS. 19A-19C) or BCR-ABL1-driven (FIGS. 19D-19F) Ctnnb1^{ex3fl/+} B-ALL cells were transduced with GFP-tagged Cre-ER^{T2} or ER^{T2} constructs. Changes of percentages of GFP⁺ cells were monitored for 8 days following 4-OHT addition. Data is a representative of three independent experiments (n=3). Ctnnb1^{ex3fl/+} NRASG^{12D} (FIG. 19B) or BCR-ABL1 (FIG. 19E) transformed B-ALL cells carrying Cre-ER^{T2}or ER^{T2} constructs were plated for colony formation assays 2 days after 4-OHT treatment. Representative images are shown for 10 days after plating. Graphs depict pooled data from two independent experiments and number of colonies formed upon β-catenin activation (Cre-ER^{T2}) were normalized to control cells (ER^{T2}). Cell cycle phases of Ctnnb1^{ex3fl/+} NRASG^{12D} (FIG. 19C) or BCR-ABL1 (FIG. 19F) transformed B-ALL cells carrying Cre-ER^{T2} or ER^{T2} vectors were measured by EdU incorporation in combination with DAPI staining 2 days after upon-catenin induction. Data shown is a representative of two independent experiments (n=3). FIG. 19G: To validate the β-catenin mediated transcriptional changes, FACS dot plots of double staining for CD19 with CD5, CD25, Ccr2 and CD244 (2B4) 0-3 days after 4-OHT-mediated induction of Cre-ER^{T2}. Numbers in FACS plots denote mean fluorescence intensities. FIG. 19H: Ctnnb1^{ex3fl/+} B-ALL cells with Cre-ER^{T2} or ER^{T2} constructs were transduced with GFP-tagged Myc, Myc^{T58A} or EV. Western blot analyses were performed for β-catenin, global Myc, Myc-pT⁵⁸, Myc-pS⁶² on FACS-sorted GFP⁺ cells 3 days after β-catenin induction.

FIGS. 20A-20E show that oncogenic β-catenin-activation has deleterious effects in lymphoid but not other lineages. Human lymphoid, myeloid and epithelial cancer cell lines or patient derived xenografts were transduced with Tet-3G activator, then with GFP-tagged, constitutively active β-catenin harboring point mutations of GSK3β-phosphorylation sites (CTNNB1) or empty vector (EV). FIGS. 5A-5B: Doxycycline was added to induce expression of β-catenin and GFP. Changes in the percentages of GFP⁺ cells were monitored by flow cytometry at the indicated time points and normalized to the frequencies GFP⁺ cells on day 0. Fold changes in GFP⁺ cells upon β-catenin accumulation were normalized to control cells (EV). Representative FACS plots are shown in FIG. 21. Data shown is a representative of two independent experiments with each three replicates. FIG. 20C: One day after doxycycline-treatment, GFP⁺ cells were flow-sorted (99.8% pure) and plated on methylcellulose medium. Colonies were imaged and counted 14 days after plating. Colony forming capacity of cells expressing CTNNB1 was calculated by normalizing to the number of colonies generated by control cells (EV). Representative images from two independent experiments are shown (n=3; FIG. 21). Cell lines left to right: BV173 (B-ALL), Z138 (B-NHL), Jurkat (T-ALL) and MV4-11 (AML). FIG. 20D: Cell cycle analyses were performed by measuring EdU incorporation 2 days after doxycycline-mediated activation of β-catenin. Changes in frequencies of cells in S-phase were plotted relative to control cells. Data shown is a representative of two independent experiments with two replicates each. Cell lines left to right: BV173 (B-ALL), JEKO (B-NHL), Jurkat (T-ALL), MV4-11 (AML) and SW480 (Colon). FIG. 20E: Annexin V and DAPI staining was performed to measure the frequencies of viable cells following β-catenin activation. Cell viabilities at each time point were normalized to cell viabilities at day 0. Data shown are representative of two independent experiments.

FIGS. 21A-21C show that genetic hyperactivation of β-catenin suppresses human lymphoid malignancies. Human lymphoid, myeloid and epithelial cell lines or PDXs were engineered to express stabilized β-catenin (CTNNB1) or empty vector (EV) together with GFP in a doxycycline dependent manner. FIG. 21A: Expansion or depletion of β-catenin expressing cells were analyzed FACs and frequencies GFP⁺ cells were normalized to the values measured at the start of doxycycline treatment. Representative FACS plots and growth kinetics from two independent experiments are shown (n=3). FIG. 21B: Western blot was performed to confirm the expression of β-catenin in AML (MV-4-11), colon cancer (SW480) and B-ALL (BLQ5) cell lines 0-2 days after treatment with doxycycline. FIG. 21C: Western blot measuring β-catenin, MYC and β-actin levels in cells treated with doxycycline for two days. 1 day after doxycycline treatment, GFP⁺ cells were sorted (99.8% pure) and plated on methylcellulose medium. Colonies were imaged and counted 14 days after plating

FIGS. 22A-22B show that Ikzf1 and Ikzf3 deletion rescues deleterious effects of β-catenin activation in B-ALL cells. BCR-ABL1-transformed Ctnnb1^{ex3fl/+} B-ALL cells were electroporated with Cas9-RNPs in complex with non-targeting crRNAs (gNT) or crRNAs targeting Ikzf1 and Ikzf3. Deletion of both Ikzf1 and Ikzf3 was confirmed by Western blot in clonal cell lines that grew out from single cells. FIG. 22A: Changes in the β-catenin interactome upon Ikzf1 and Ikzf3 deletion were analyzed by co-IP and mass-spectrometry. Proteins bound to β-catenin were plotted based on significance (y-axis) and log 2-fold enrichment (x-axis) over the control (n=3). FIG. 22B: BCR-ABL1-transformed Ctnnb1^{ex3fl/+} B-ALL cells that were previously transduced with GFP-tagged, 4-OHT inducible Cre-ER^{T2} (β-catenin^{GOF}) or ER^{T2} were transduced with red fluorescent protein (RFP)-tagged Foxp1 or control vector (EV). Increase or decrease of GFP⁺ cells within the RFP⁺ compartment was analyzed by FACS for 6 days after β-catenin activation.

FIGS. 23A-23E show that β-catenin negatively regulates human lymphopoiesis but not myeloid cell expansion. Human CD34⁺ cord blood HSCs were edited with non-targeting controls (gNT) or guides targeting Ctnnb1 (gCTNNB1) and injected into NSGW41 mice. Data shown is a pool of two independent transplant experiments with two different cord blood donors; n=6. FIG. 23A: Bone marrow samples were analyzed for presence of human leukocytes (hCD45⁺), hematopoietic stem (CD34⁺ CD38⁻) and progenitor cells (CD34⁺ CD38⁺), myeloid cells (CD33⁺), B-cells (CD19⁺) and T-cells (CD3⁺) 15 weeks after engraftment. Frequencies of human leukocytes (CD45⁺) cells in the blood of NSGW41 mice are shown for 10 and 15 weeks after transplantation. FIG. 23B: Representative FACS plots and numbers of human pro-B cells (CD10⁺ CD19⁺ CD34⁺), pre-B cells (CD10⁺ CD19⁺ CD34⁻ IgM⁻), immature B-cells (CD10⁺ CD19⁺ IgM⁺) and mature B-cells (CD10⁻ CD19⁺ IgM⁺ IgD⁺) in the bone marrows of NSGW41 mice. FIG. 23C: Images and total cell numbers are shown from spleens (left) and thymi (right) of NSGW41 mice humanized with hematopoietic progenitor cells with and without CTNNB1-deletion. FIG. 23D: Representative FACS plots and cell numbers of human splenic leukocytes (hCD45⁺), myeloid (CD33⁺), B-cells (CD19⁺: IgM⁻ IgD⁻ immature, IgM⁺ IgD⁻ and IgM⁺ IgD⁺ mature B cells) and T-cells (CD3⁺). FIG. 23E: Representative flow cytometry analyses and absolute cell numbers of human pro-T cells (CD7⁺ CD34⁺), CD4⁺ CD8⁺ double positive thymocytes (DP), CD4⁺ or CD8⁺ single positive T-cells in the thymus of NSGW41 mice.

FIGS. 24A-24C show that engineered deletion of CTNNB1 improves lymphopoiesis from MDS progenitor cells. ssDNA repair template-mediated deletion of β-catenin was performed and CD34⁺ HDRT-GFP⁺ bone marrow progenitor cells from an MDS-patient were flow sorted (FIG. 24A) for transplantation into MISTRG mice. After 16 weeks, human multi-lineage reconstitution was assessed by flow cytometry, demonstrating increased human chimerism and enhanced B-lymphopoiesis from CTNNB1^{-/-} progenitor cells (FIGS. 24B-24C). Strikingly, MDS bone marrow progenitor cells were not able to produce T-cells unless CTNNB1 was deleted.

FIGS. 25A-25D show that negative regulation of β-catenin is essential for early B-cell development. β-catenin residues S33 and S37 (Exon 3) are phosphorylated by GSK3β for β-catenin-degradation. B-cell-specific expression of Cre and excision of exon 3 (Mbl-Cre) prevents GSK3β-mediated degradation of β-catenin and results in profound depletion of B-cells in vivo (FIG. 25A) and in vitro (FIG. 25B). The pool of mature B-cells in the spleen was drastically reduced (FIG. 25C). Flow cytometry analyses of early B-cell development in the bone marrow (FIG. 25D) revealed a profound B-cell defect from Hardy Fractions C and C′.

FIG. 26 shows that pathological BCR-signaling induces nuclear β-catenin accumulation in autoreactive B-cells. As a classical model for anergy and clonal deletion of autoreactive B-cells, Ig^{HEL} mice were crossed ML5 mice that express soluble HEL (sHEL). Splenic Ig^{HEL} B-cells in the presence of sHEL for12 hours induced anergic phenotypes and cell cycle exit. Pathological BCR signaling upon chronic persistent exposure to self-antigen induces phosphorylation and nuclear accumulation of β-catenin and repression of Myc.

FIG. 27 shows that β-catenin accumulation functions as sensor for pathological BCR-signaling in autoreactive B-cells. Ctnnb1^{+/+} and Ctnnb1^{ex3fl/f1} mice crossed with tamoxifen-inducible Mb1-CreER^{T2}. Upon inducible β-catenin accumulation, splenic B-cells upregulated IgD at the expense of IgM, transitional B-cells acquired a T3 phenotype that is typically enriched for autoreactive B-cells with massive downregulation of IgM and upregulation of CD23. As a functional readout of B-cell anergy, β-catenin accumulation suppressed responsiveness of the BCR, measured as loss of calcium flux.

FIGS. 28A-28C show that GSK3β small molecule inhibitors selectively kill B-cell lines by β-catenin-Ikaros-mediated MYC repression. Pre-B cell and mature B-cell lines as well as colon and lung cancer cell lines were treated with two FDA-approved GSK3β small molecule inhibitors LY2090314 (FIGS. 28A-28B) and CHIR99021 (FIG. 28C). B-cell lines express β-catenin at very low baseline levels. However, GSK3β-inhibition induced rapid accumulation of β-catenin protein and suppression of MYC in Ikaros-expressing B-cell lines but not colon and lung cell lines lacking Ikaros expression (FIG. 28A). B-cell lines of pre-B cell and mature B-cell origin had IC₅₀ values for the CHIR99021 GSK3β small molecule inhibitor that were 307-436-fold lower than colon and lung cancer cell lines (FIG. 28C).

FIG. 29 shows defective central B-cell tolerance in humanized mice engrafted with HSCs from SLE and RA patients. Humanized mice engrafted with HSCs isolated from the bone marrow of four patients with SLE and four patients with RA were generated. Mice engrafted with patients' HSCs generated elevated frequencies of autoreactive B-cells compared to mice engrafted with HSCs from health donors, similar to those in the blood of patients and healthy donors. Frequencies of polyreactive clones in new emigrant and transitional B-cells isolated from the blood of patients or the spleen of humanized mice are shown (HD: healthy donors). Each symbol represents one patient sample, studied in humanized mice.

FIG. 30 depicts exemplary graphical representation showing that in B-cells, instead of MYC-activation as in other cells, β-catenin was essential to enable Ikaros-mediated recruitment of nucleosome remodeling and deacetylation (NuRD) complexes for transcriptional repression of MYC.

### DETAILED DESCRIPTION

Oncogenic activation of Wnt/β-catenin signaling is common throughout all types of cancer. In striking contrast, it was found in the present disclosure that lymphoid malignancies are not only exempt from activating Wnt/β-catenin lesions but are highly sensitive to oncogenic activation of β-catenin: Unlike other cell types, inducible activation of β-catenin in human lymphoid malignancies cells, suppressed MYC-expression, cell proliferation, colony formation and induced cell death. The global interactome studies of the present disclosure in lymphoid malignancies cells revealed repressive β-catenin complexes with lymphoid-specific Ikaros zinc finger (IKZF) proteins that were responsible for lymphoid-specific toxicity of β-catenin activation.

To leverage β-catenin:IKZF complexes as previously unrecognized therapeutic vulnerability in lymphoid malignancies and other lymphocyte associated diseases or conditions (e.g., autoimmune diseases, graft versus host disease, etc.), inhibition of GSK3p, a central negative regulator of β-catenin, proved to be effective as indicated in the Examples. Strikingly, as disclosed herein, small molecule GSK3β-inhibitors such as those used in clinical trials for the treatment of solid tumors, were effective at low nanomolar concentrations (e.g., in patient-derived xenografts (PDX) from lymphoid malignancies PDX, induced massive accumulation of β-catenin, repression of MYC, and acute cell death. Importantly, four GSK3β-inhibitors have already undergone full clinical development and demonstrated favorable safety profiles in phase 1 and 2 trials for solid tumors (18 trials, 11 cancer types). In these trials, GSK3β-inhibitors were used to reach micromolar serum concentrations but failed to achieve clinical responses. Given that prolonged treatment at >100-fold higher concentrations than what is needed to elicit therapeutic responses in lymphoid malignancies has proven to be safe in these trials, it is proposed herein to repurpose existing GSK3β-inhibitors for the treatment of refractory lymphoid malignancies based on targeted engagement of repressive β-catenin:IKZF1 complexes. Experiments based on patient-derived xenografts validated GSK3β-inhibitors for targeted engagement of lymphoid β-catenin:IKZF complexes in vivo as a novel strategy. In certain embodiments, GSK3β-inhibitors as also useful to overcome drug-resistance in refractory lymphoid malignancies. In certain embodiments, GSK3β-inhibitors as also useful to treat patients that have relapsed. In certain embodiments, GSK3β-inhibitors as also useful in a combination treatment (e.g., to reduce the dosage amount of the non-GSK3β inhibitor and/or enhance the effectiveness of the non-GSK3β inhibitor).

Given that that IKZF1, TKZF2 and IKZF3 are only expressed and active in lymphoid cells, thus, the present invention leverages a unique vulnerability of associated diseases or conditions (e.g., lymphoid malignancies, autoimmune diseases, graft versus host disease, etc.). It is particular advantageous that GSK3β inhibitors are effective in low nanomolar ranges in these diseases and conditions, while having essential no effects in any other cell types.

In most cell types, nuclear β-catenin functions as prominent oncogenic driver and pairs with TCF7-family factors for transcriptional activation of MYC. Surprisingly, B-lymphoid malignancies not only lacked expression and activating lesions of β-catenin but critically depended on GSK3β3 for effective β-catenin degradation. The present interactome studies in B-lymphoid tumors revealed that β-catenin formed repressive complexes with lymphoid-specific Ikaros factors at the expense of TCF7. Instead of MYC-activation, β-catenin was essential to enable Ikaros-mediated recruitment of nucleosome remodeling and deacetylation (NuRD) complexes for transcriptional repression of MYC.

To leverage this previously unrecognized vulnerability of B-cell-specific repressive β-catenin-Ikaros-complexes in refractory B-cell malignancies, GSK3β3 small molecule inhibitors were examined to subvert β-catenin degradation. Clinically approved GSK3β0-inhibitors that achieved favorable safety profiles at micromolar concentrations in clinical trials for neurological disorders and solid tumors were effective at low nanomolar concentrations in B-cell malignancies, induced massive accumulation of β-catenin, repression of MYC and acute cell death. Preclinical in vivo treatment experiments in patient-derived xenografts validated small molecule GSK3β-inhibitors for targeted engagement of lymphoid-specific β-catenin-Ikaros complexes as a novel strategy to overcome conventional mechanisms of drug-resistance in refractory B-cell malignancies.

The present disclosure also demonstrated that: unlike other cell lineages, B-cells express nuclear β-catenin protein at low baseline levels and depend on GSK3β for its degradation; in B-cells, β-catenin forms unique complexes with lymphoid-specific Ikaros factors and is required for Ikaros-mediated tumor suppression and assembly of repressive NuRD complexes; CRISPR-based knockin mutation of a single Ikaros-binding motif in a lymphoid MYC superenhancer region reversed β-catenin-dependent Myc repression and induction of cell death; the discovery of GSK3β-dependent degradation of β-catenin as unique B-lymphoid vulnerability provides a rationale to repurpose clinically approved GSK3β-inhibitors for the treatment of refractory B-cell malignancies.

### Definitions

Unless defined otherwise, technical and scientific terms used herein have the same meaning as commonly understood by one of ordinary skill in the art to which this invention belongs.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The terms “patient”, “individual”, and “subject”, are used interchangeably herein and refer to mammals, including, without limitation, human and veterinary animals (e.g., cats, dogs, cows, horses, sheep, pigs, etc.) and experimental animal models. In a preferred embodiment, the subject is a human.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof, or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The terms “therapeutically effective amount” and “effective amount” are used interchangeably herein to refer to the administration of an agent to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like.

The term “inhibit”, “inhibitor”, “suppress” or “suppressor”, with respect to a biological activity or process (e.g., β-catenin activation or β-catenin:IKZF complex formation), refers to a decrease in the biological activity or basal activity of the biological process.

The term “relapse” is used herein to refer to the return of a disease or the signs and symptoms of a disease after a period of improvement or remission.

As used in this disclosure and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

### β-Catenin:Ikaros Zinc Finger (IKZF) Protein Complex Agents

In some embodiments, the agonist or activator of the β-catenin:IKZF protein complex can be an agent that inhibits the expression or function of Glycogen Synthase Kinase 33 (GSK30), Axis Inhibition Protein 1 (AXIN1), Axis Inhibition Protein 2 (AXIN2), Adenomatous Polyposis Coli (APC), and/or beta-transducin repeat containing (beta-TCRP).

In some embodiments, the agonist or activator of the β-catenin:IKZF protein complex can be a small molecule, a peptide, an antibody or functional fragment thereof, an siRNA, an shRNA, a gapmer, an antisense oligonucleotide, an aptamer, a site-specific nuclease, or a proteolysis targeting chimeric (PROTAC)-degrader.

In some embodiments, the agent that inhibits the expression or function of GSK3pR is a GSK3β inhibitor.

In some embodiments, the GSK3β inhibitor can be a small molecule, a peptide, an antibody or functional fragment thereof, an siRNA, an shRNA, a gapmer, an antisense oligonucleotide, an aptamer, a site-specific nuclease, or a proteolysis targeting chimeric (PROTAC)-degrader.

In some embodiments, examples of the GSK3β inhibitor small molecule include, but are not limited to, KY19382 (A3051), 2-D08 (2′,3′,4′-trihydroxy flavone), TWS119, AR-A014418 (GSK-30 Inhibitor VIII), IM-12, AT7519, Indirubin (NSC 105327), TDZD-8 (NP 01139), MAZ51, CP21R7 (CP21), Resibufogenin (Bufogenin, Recibufogenin), Alsterpaullone (Alp, 9-Nitropaullone, NSC 705701), BIO-acetoxime (GSK-3 Inhibitor X), 1-Azakenpaullone (1-Akp), AZD1080, SB216763, SB415286, BRD0705, a diazepinoindole-based molecule, a biindole-based molecule, an aminopyrimidine-based molecule, a thiadiazolidine-based molecule, or a maleimide-based molecule.

In some embodiments, the small molecule can be a diazepinoindole, a biindole, an aminopyrimidine, a thiadiazolidine or a maleimide-based molecule.

In some embodiments, the small molecule can be a diazepinoindole, a biindole, or an aminopyrimidine.

In some embodiments, the diazepinoindole can be LY2090314. In some embodiments, the small molecule can be a diazepinoindole molecules as described in WO 2009/006043, herein incorporated by reference in its entirety for all purposes

In some embodiments, the biindole can be 6-Bromoindirubin-3′-oxime.

In some embodiments, the aminopyrimidine can be CHIR98014 or CHIR99021.

In some embodiments, the thiadiazolidine can be Tideglusib.

In some embodiments, the maleimide-based molecule can be 9-ING-41.

In some embodiments, the agent that inhibits the expression or function of β-catenin or a β-catenin:IKZF protein complex is a small molecule, a peptide, an antibody or antibody fragment, an siRNA, an shRNA, a gapmer, an antisense oligonucleotide, an aptamer, PROTAC-degrader, or a site-specific nuclease.

In some embodiments, the β-catenin gene is knocked out or knocked down.

Broadly, an “antibody” refers to a polypeptide or protein that consists of or comprises antibody domains, which are understood as constant and/or variable domains of the heavy and/or light chains of immunoglobulins, with or without a linker sequence. In an embodiment, polypeptides are understood as antibody domains if they comprise a beta-barrel sequence consisting of at least two beta-strands of an antibody domain structure connected by a loop sequence. Antibody domains may be of native structure or modified by mutagenesis or derivatization, e.g., to modify binding specificity or any other property.

The term “antibody” refers to an intact antibody. In an embodiment, an “antibody” may comprise a complete (i.e., full-length) immunoglobulin molecule, including e.g., polyclonal, monoclonal, chimeric, humanized and/or human versions having full length heavy and/or light chains. The term “antibody” encompasses any and all isotypes and subclasses, including without limitation the major classes of IgA, IgD, IgE, IgG and IgM, and the subclasses IgG1, IgG2, IgG3, IgG4, IgAQ1 and IgA2. In an embodiment, the antibody is an IgG. The antibody may be one that is naturally occurring or one that is prepared by any means available to the skilled person, such as for example by using animals or hybridomas, and/or by immunoglobulin gene fragment recombinatorial processes.

The antibody may be of any origin, including natural, recombinant and/or synthetic sources. In an embodiment, the antibody may be of animal origin. In an embodiment, the antibody may be of mammalian origin, including without limitation human, murine, rabbit and goat. In an embodiment, the antibody may be a recombinant antibody.

In an embodiment, the antibody may be a monoclonal antibody, a polyclonal antibody, a chimeric antibody, a humanized antibody, a human antibody or a fully human antibody. The meaning applied to these terms and the types of antibodies encompassed therein will be well understood by the skilled person.

As used herein, the term “functional fragment”, with respect to an antibody, refers to an antigen-binding portion of an antibody. In this context, by “functional” it is meant that the fragment maintains its ability to bind to the target antigen. In an embodiment, the binding affinity may be equivalent to, or greater than, that of parent antibody. In an embodiment, the binding affinity may be less than the parent antibody, but nevertheless the functional fragment maintains a specificity and/or selectivity for the target antigen.

Functional fragments of antibodies include, without limitation, a portion of an antibody such as a F(ab′)₂, a F(ab)₂, a Fab′, a Fab, a Fab₂, a Fab₃, a single domain antibody (e.g., a Dab or VHHs) and the like, including half-molecules of IgG4 (van der Neut Kolfschoten, 2007). Regardless of structure, a functional fragment of an antibody binds with the same antigen that is recognized by the intact antibody. The term “functional fragment”, in relation to antibodies, also includes isolated fragments consisting of the variable regions, such as the “Fv” fragments consisting of the variable regions of the heavy and light chains and recombinant single chain polypeptide molecules in which light and heavy chain variable regions are connected by a peptide linker (“scFv proteins”). As used herein, the term “functional fragment” does not include fragments such as Fc fragments that do not contain antigen-binding sites.

Antibody fragments, such as those described herein, can be incorporated into single domain antibodies (e.g., nanobodies), single-chain antibodies, maxibodies, evibodies, minibodies, intrabodies, diabodies, triabodies, tetrabodies, vNAR, bis-scFv and other like structures (see e.g., Hollinger and Hudson, 2005). Antibody polypeptides including fibronectin polypeptide monobodies, also are disclosed in U.S. Pat. No. 6,703,199. Other antibody polypeptides are disclosed in U.S. Patent Publication No. 20050238646. Each reference cited herein is incorporated by reference in their entirety for all purposes.

Another form of a functional fragment is a peptide comprising one or more CDRs of an antibody or one or more portions of the CDRs, provided the resultant peptide retains the ability to bind the target antigen.

A functional fragment may be a synthetic or genetically engineer protein. For example, functional fragments include isolated fragments consisting of the light chain variable region, “Fv” fragments consisting of the variable regions of the heavy and light chains, and recombinant single chain polypeptide molecules which light and heavy regions are connected by a peptide linker (scFv proteins)

In some embodiments, the GSK3β inhibitor can be an inhibitory oligonucleotide. In some embodiments, the inhibitory oligonucleotide can be, but not limited to, a double-stranded RNA (dsRNA), a small hairpin RNA (shRNA), a small interfering RNA (siRNA), a microRNA (miRNA), a Piwi-interacting RNA (piRNA), a ribozyme, a long non-coding RNA (lncRNA), an antisense RNAs, or a RNAse external guide sequences (EGSs).

In some embodiments, the site-specific nuclease can be an engineered homing endo-nuclease or meganuclease, a zinc-finger nucleases (ZFNs), a transcription activator-like effector nucleases (TALENs), or a clustered regularly interspaced short palindromic repeat (CRISPR) system. In some embodiments, the GSK3β inhibitor can be a gene-editing molecule.

The methods disclosed herein can utilize the Clustered Regularly Interspersed Short Palindromic Repeats (CRISPR)/CRISPR-associated (Cas) systems or components of such systems to modify a genome within a cell. CRISPR/Cas systems include transcripts and other elements involved in the expression of, or directing the activity of, Cas genes. A CRISPR/Cas system can be, for example, a type I, a type II, or a type III system. Alternatively, a CRISPR/Cas system can be a type V system (e.g., subtype V-A or subtype V-B). The methods disclosed herein can employ CRISPR/Cas systems by utilizing CRISPR complexes (comprising a guide RNA (gRNA) complexed with a Cas protein) for site-directed cleavage of nucleic acids.

CRISPR/Cas systems used in the methods disclosed herein can be non-naturally occurring. A “non-naturally occurring” system includes anything indicating the involvement of the hand of man, such as one or more components of the system being altered or mutated from their naturally occurring state, being at least substantially free from at least one other component with which they are naturally associated in nature, or being associated with at least one other component with which they are not naturally associated. For example, some CRISPR/Cas systems employ non-naturally occurring CRISPR complexes comprising a gRNA and a Cas protein that do not naturally occur together, employ a Cas protein that does not occur naturally, or employ a gRNA that does not occur naturally.

“Cas molecules”, “Cas proteins” or “Cas nucleases” useful in the compositions and methods of the invention generally comprise at least one RNA recognition or binding domain that can interact with guide RNAs (gRNAs, described in more detail below). Cas proteins can also comprise nuclease domains (e.g., DNase or RNase domains), DNA binding domains, helicase domains, protein-protein interaction domains, dimerization domains, and other domains. A nuclease domain possesses catalytic activity for nucleic acid cleavage, which includes the breakage of the covalent bonds of a nucleic acid molecule. Cleavage can produce blunt ends or staggered ends, and it can be single-stranded or double-stranded. For example, a wild type Cas9 protein will typically create a blunt cleavage product. Alternatively, a wild type Cpf1 protein (e.g., FnCpf1) can result in a cleavage product with a 5-nucleotide 5′ overhang, with the cleavage occurring after the 18th base pair from the PAM sequence on the non-targeted strand and after the 23rd base on the targeted strand. A Cas protein can have full cleavage activity to create a double-strand break at a target genomic locus (e.g., a double-strand break with blunt ends), or it can be a nickase that creates a single-strand break at a target genomic locus.

Examples of Cas proteins useful in the methods of the invention include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

An exemplary Cas protein is a Cas9 protein or a protein derived from Cas9 from a type II CRISPR/Cas system. Cas9 proteins are from a type II CRISPR/Cas system and typically share four key motifs with a conserved architecture. Motifs 1, 2, and 4 are RuvC-like motifs, and motif 3 is an HNH motif. Exemplary Cas9 proteins are from Streptococcus pyogenes, Streptococcus thermophilus, Streptococcus sp., Staphylococcus aureus, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Neisseria meningitidis, or Campylobacter jejuni. Additional examples of the Cas9 family members are described in WO 2014/131833, herein incorporated by reference in its entirety for all purposes. Cas9 from S. pyogenes (SpCas9) (assigned SwissProt accession number Q99ZW2) is an exemplary Cas9 protein. Cas9 from S. aureus (SaCas9) (assigned UniProt accession number J7RUA5) is another exemplary Cas9 protein. Cas9 from Campylobacter jejuni (CjCas9) (assigned UniProt accession number Q0P897) is another exemplary Cas9 protein. See, e.g., Kim et al. (2017) Nat. Comm. 8:14500, herein incorporated by reference in its entirety for all purposes. SaCas9 is smaller than SpCas9, and CjCas9 is smaller than both SaCas9 and SpCas9.

Another example of a Cas protein is a Cpf1 (CRISPR from Prevotella and Francisella 1) protein. Cpf1 is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in Cas9 proteins, and the RuvC-like domain is contiguous in the Cpf1 sequence, in contrast to Cas9 where it contains long inserts including the HNH domain. See, e.g., Zetsche et al. (2015) Cell 163(3):759-771, herein incorporated by reference in its entirety for all purposes. Exemplary Cpf1 proteins are from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. Cpf1 from Francisella novicida U112 (FnCpf1; assigned UniProt accession number AOQ7Q2) is an exemplary Cpf1 protein.

Cas proteins can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins. Active variants or fragments with respect to catalytic activity can comprise at least 80%, 85%, 90%, 91%, 92%, 93%, 94%, 95%, 96%, 97%, 98%, 99% or more sequence identity to the wild type or modified Cas protein or a portion thereof, wherein the active variants retain the ability to cut at a desired cleavage site and hence retain nick-inducing or double-strand-break-inducing activity. Assays for nick-inducing or double-strand-break-inducing activity are known and generally measure the overall activity and specificity of the Cas protein on DNA substrates containing the cleavage site.

Cas proteins can be modified to increase or decrease one or more of nucleic acid binding affinity, nucleic acid binding specificity, and enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein.

Cas proteins can comprise at least one nuclease domain, such as a DNase domain. For example, a wild type Cpf1 protein generally comprises a RuvC-like domain that cleaves both strands of target DNA, perhaps in a dimeric configuration. Cas proteins can also comprise at least two nuclease domains, such as DNase domains. For example, a wild type Cas9 protein generally comprises a RuvC-like nuclease domain and an HNH-like nuclease domain. The RuvC and HNH domains can each cut a different strand of double-stranded DNA to make a double-stranded break in the DNA. See, e.g., Jinek et al. (2012) Science 337:816-821, herein incorporated by reference in its entirety for all purposes.

In certain embodiments, the Cas molecule is a Cas9 molecule, or a functional fragment or derivative thereof. In certain embodiments, the Cas9 can be wild type Cas9, a Cas9 nickase, a dead Cas9 (dCas9) a split Cas9, and a Cas9 fusion protein. In certain embodiments, the Cas9 is a Streptococcus pyogenes or Staphylococcus aureus Cas9. In certain embodiments, the sequence of the Cas9 mRNA is codon optimized for expression in a eukaryotic cell.

In some embodiments, the gRNA sequences used for CRISPR-mediated gene modification is GCGAGGTATTCGGCTCCGCG (SEQ ID NO: 1) (non-targeting control gRNA). In some embodiments, the gRNA sequences used for CRISPR-mediated gene modification is ACAATGGCAGACACCATCTG (SEQ ID NO: 2) (mouse Ctnnb1 deletion gRNA). In some embodiments, the gRNA sequences used for CRISPR-mediated gene modification is CTGGAGTGTCACTGACTGGG (SEQ ID NO: 3) (mouse Ikzf1 deletion gRNA). In some embodiments, the gRNA sequences used for CRISPR-mediated gene modification is ATTATGAAGCCGGAGCCCAT (SEQ ID NO: 4) (mouse Ikzf3 deletion gRNA target). In some embodiments, the gRNA sequences used for CRISPR-mediated gene modification is GGTTCTTGACTACCGTAATT (SEQ ID NO: 5) (non-targeting control gRNA). In some embodiments, the gRNA sequences used for CRISPR-mediated gene modification is AAGGTTATGCAAGGTCCCAG (SEQ ID NO: 6) (human CTNNB1 deletion gRNA).

Transcription activator-like effector nucleases (TALEN) are restriction enzymes that can be engineered to cut target sequences of DNA. They are made by fusing a TAL effector DNA-binding domain to a DNA cleavage domain (a nuclease which cuts DNA strands). TAL effector nucleases are a class of sequence-specific nucleases that can be used to make double-strand breaks at specific target sequences in the genome of a prokaryotic or eukaryotic organism. TAL effector nucleases are created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas.1013133107; Scholze & Boch (2010) Virulence 1:428-432; Christian et al. Genetics (2010) 186:757-761; Li et al. (2010) Nuc. Acids Res. doi: 10.1093/nar/gkg704; and Miller et al. (2011) Nature Biotechnology 29:143-148; all of which are herein incorporated by reference in their entirety and for all purposes.

Examples of suitable TAL nucleases, and methods for preparing suitable TAL nucleases, are disclosed, e.g., in US Patent Application No. 2011/0239315 A1, 2011/0269234 A1, 2011/0145940 A1, 2003/0232410 A1, 2005/0208489 A1, 2005/0026157 A1, 2005/0064474 A1, 2006/0188987 A1, and 2006/0063231 A1 (each hereby incorporated by reference in their entirety and for all purposes). In some embodiments, TAL effector nucleases are engineered that cut in or near a target nucleic acid sequence in, e.g., a genomic locus of interest, wherein the target nucleic acid sequence is at or near a sequence to be modified by a targeting vector. The TAL nucleases suitable for use with the various methods provided herein include those that are specifically designed to bind at or near target nucleic acid sequences to be modified by targeting vectors.

In some embodiments, each monomer of the TALEN comprises 12-25 TAL repeats, wherein each TAL repeat binds a 1 bp subsite. In certain embodiments, the gene-editing molecule is a chimeric protein comprising a TAL repeat-based DNA binding domain operably linked to an independent nuclease. In some embodiments, the independent nuclease is a FokI endonuclease. In some embodiments, the gene-editing molecule comprises a first TAL-repeat-based DNA binding domain and a second TAL-repeat-based DNA binding domain, wherein each of the first and the second TAL-repeat-based DNA binding domain is operably linked to a FokI nuclease, wherein the first and the second TAL-repeat-based DNA binding domain recognize two contiguous target DNA sequences in each strand of the target DNA sequence separated by about 6 bp to about 40 bp cleavage site, and wherein the FokI nucleases dimerize and make a double strand break at a target sequence.

In some embodiments, the gene-editing molecule comprises a first TAL-repeat-based DNA binding domain and a second TAL-repeat-based DNA binding domain, wherein each of the first and the second TAL-repeat-based DNA binding domain is operably linked to a FokI nuclease, wherein the first and the second TAL-repeat-based DNA binding domain recognize two contiguous target DNA sequences in each strand of the target DNA sequence separated by a 5 bp or 6 bp cleavage site, and wherein the FokI nucleases dimerize and make a double strand break.

The gene-editing molecule employed in the various methods and compositions disclosed herein can further comprise a zinc-finger nuclease (ZFN). Zinc finger nucleases (ZFNs) are a class of engineered DNA-binding proteins that assist targeted editing of the genome by creating double strand breaks (DSBs) in DNA at targeted locations. ZFNs comprise two functional domains: i) a DNA-binding domain comprising a chain of two-finger modules (each recognizing a unique hexamer (6 bp) sequence of DNA—two-finger modules are stitched together to form a Zinc Finger Protein, each with specificity of ≥24 bp) and ii) a DNA-cleaving domain comprising a nuclease domain of Fok I. When the DNA-binding and -cleaving domains are fused together, a highly-specific pair of “genomic scissors” are created.

In some embodiments, each monomer of the ZFN comprises 3 or more zinc finger-based DNA binding domains, wherein each zinc finger-based DNA binding domain binds to a 3 bp subsite. In other embodiments, the ZFN is a chimeric protein comprising a zinc finger-based DNA binding domain operably linked to an independent nuclease. In some embodiments, the independent endonuclease is a FokI endonuclease. In some embodiments, the gene-editing molecule comprises a first ZFN and a second ZFN, wherein each of the first ZFN and the second ZFN is operably linked to a FokI nuclease, wherein the first and the second ZFN recognize two contiguous target DNA sequences in each strand of the target DNA sequence separated by about 6 bp to about 40 bp cleavage site or about a 5 bp to about 6 bp cleavage site, and wherein the FokI nucleases dimerize and make a double strand break. See, e.g., US20060246567; US20080182332; US20020081614; US20030021776; WO/2002/057308A2; US20130123484; US20100291048; and, WO/2011/017293A2, each of which is herein incorporated by reference in their entirety for all purposes.

In some embodiments of the compositions and methods provided herein, the gene-editing molecule comprises (a) a chimeric protein comprising a zinc finger-based DNA binding domain fused to a FokI endonuclease; or (b) a chimeric protein comprising a Transcription Activator-Like Effector Nuclease (TALEN) fused to a FokI endonuclease.

In still another embodiment, the gene-editing molecule is a meganuclease. Meganucleases have been classified into four families based on conserved sequence motifs, the families are the LAGLIDADG (SEQ ID NO: 7), GIY-YIG, H-N-H, and His-Cys box families. These motifs participate in the coordination of metal ions and hydrolysis of phosphodiester bonds. HEases are notable for their long recognition sites, and for tolerating some sequence polymorphisms in their DNA substrates. Meganuclease domains, structure and function are known, see e.g., Guhan and Muniyappa (2003) Crit Rev Biochem Mol Biol 38:199-248; Lucas et al., (2001) Nucleic Acids Res 29:960-9; Jurica and Stoddard, (1999) Cell Mol Life Sci 55:1304-26; Stoddard, (2006) Q Rev Biophys 38:49-95; and Moure et al., (2002) Nat Struct Biol 9:764. In some examples a naturally occurring variant, and/or engineered derivative meganuclease is used. Methods for modifying the kinetics, cofactor interactions, expression, optimal conditions, and/or recognition site specificity, and screening for activity are known, see e.g., Epinat et al., (2003) Nucleic Acids Res 31:2952-62; Chevalier et al., (2002) Mol Cell 10:895-905; Gimble et al., (2003) Mol Biol 334:993-1008; Seligman et al., (2002) Nucleic Acids Res 30:3870-9; Sussman et al., (2004) J Mol Biol 342:31-41; Rosen et al., (2006) Nucleic Acids Res 34:4791-800; Chames et al., (2005) Nucleic Acids Res 33:e178; Smith et al., (2006) Nucleic Acids Res 34:e149; Gruen et al., (2002) Nucleic Acids Res 30:e29; Chen and Zhao, (2005) Nucleic Acids Res 33:e154; WO2005105989; WO2003078619; WO2006097854; WO2006097853; WO2006097784; and WO2004031346.

Any meganuclease can be used herein, including, but not limited to, I-SceI, I-SceII, I-SceIII, I-SceIV, I-SceV, I-SceVI, I-SceVII, I-CeuI, I-CeuAIIP, I-CreI, I-CrepsbIP, I-CrepsbIIP, I-CrepsbIIIP, I-CrepsbIVP, I-TliI, I-PpoI, PI-PspI, F-SceI, F-SceII, F-SuvI, F-TevI, F-TevII, I-Aural, I-Anil, I-ChuI, I-CmoeI, I-CpaI, I-CpaII, I-CsmI, I-CvuI, I-CvuAIP, I-DdiI, I-DdiII, I-DirI, I-DmoI, I-HmuI, I-HmuII, I-HsNIP, I-LlaI, I-MsoI, I-NaaI, I-NanI, I-NcIIP, I-NgrIP, I-NitI, I-NjaI, I-Nsp236IP, I-PakI, I-PboIP, I-PcuIP, I-PcuAI, I-PcuVI, I-PgrIP, I-PobIP, I-PorI, I-PorIIP, I-PbpIP, I-SpBetaIP, I-ScaI, I-SexIP, I-SneIP, I-SpomI, I-SpomCP, I-SpomIP, I-SpomIIP, I-SquIP, I-Ssp6803I, I-SthPhiJP, I-SthPhiST3P, I-SthPhiSTe3bP, I-TdeIP, I-TevI, I-TevII, I-TevIII, I-UarAP, I-UarHGPAIP, I-UarHGPA13P, I-VinIP, I-ZbiIP, PI-MtuI, PI-MtuHIP PI-MtuHIIP, PI-PfuI, PI-PfuII, PI-PkoI, PI-PkoII, PI-Rma43812IP, PI-SpBetaIP, PI-SceI, PI-TfuI, PI-TfuII, PI-ThyI, PI-TliI, PI-TliII, or any active variants or fragments thereof.

In one embodiment, the meganuclease recognizes double-stranded DNA sequences of 12 to 40 base pairs. In one embodiment, the meganuclease recognizes one perfectly matched target sequence in the genome. In one embodiment, the meganuclease is a homing nuclease. In one embodiment, the homing nuclease is a LAGLIDADG (SEQ ID NO: 7) family of homing nuclease. In one embodiment, the LAGLIDADG (SEQ ID NO: 7) family of homing nuclease is selected from I-SceI, I-CreI, and I-Dmol.

ZFNs and TALENs introduce DSBs in a target genomic sequence and activate non-homologous end-joining (NHEJ)-mediated DNA repair, which generates a mutant allele comprising an insertion or a deletion of a nucleic acid sequence at the genomic locus of interest and thereby causes disruption of the genomic locus of interest in a cell. DSBs also stimulate homology-directed repair (HDR) by homologous recombination if a repair template is provided. HDR can result in a perfect repair that restores the original sequence at the broken site, or it can be used to direct a designed modification, such as a deletion, insertion, or replacement of the sequence at the site of the double strand break.

In some embodiments, the GSK3β inhibitor can be administered to the subject a dose sufficient to result in a steady state plasma concentration of the inhibitor from about 0.1 nM to about 900 nM, about 1 nM to about 750 nM, about 5 nM to about 500 nM, about 10 nM to about 400 nM, about 20 nM to about 300 nM, about 30 nM to about 250 nM, about 40 nM to about 200 nM, or about 50 nM to about 100 nM. In some embodiments, the GSK3β inhibitor can be administered to the subject a dose sufficient to result in a steady state plasma concentration of the inhibitor from about 1 nM to about 100 nM, from about 1 nM to about 200 nM, from about 1 nM to about 300 nM, from about 1 nM to about 400 nM, from about 1 nM to about 500 nM, about 5 nM to about 100 nM, from about 5 nM to about 200 nM, from about 5 nM to about 300 nM, from about 5 nM to about 400 nM, from about 5 nM to about 500 nM, about 10 nM to about 100 nM, from about 10 nM to about 200 nM, from about 10 nM to about 300 nM, from about 10 nM to about 400 nM, or from about 10 nM to about 500 nM. In some embodiments, the GSK3β inhibitor can be administered to the subject a dose sufficient to result in a steady state plasma concentration of the inhibitor from about 5 nM to about 500 nM.

In various embodiments, the GSK3β inhibitor can be administered to the subject a dose sufficient to result in a steady state plasma concentration of the inhibitor of about, at least about, or no more than about 0.1 nM, 0.2 mM, 0.3 nM, 0.4 nM, 0.5 nM, 0.6 nM, 0.7 mM, 0.8 nM, 0.9 nM, 1 nM, 1.5 nM, 2 nM, 2.5 nM, 3 nM, 3.5 nM, 4 nM, 4.5 nM, 5 nM, 10 nM, 15 nM, 20 nM, 25 nM, 30 nM, 35 nM, 40 nM, 45 nM, 50 nM, 55 nM, 60 nM, 65 nM, 70 nM, 75 nM, 80 nM, 85 nM, 90 nM, 95 nM, 100 nM, 105 nM, 110 nM, 115 nM, 120 nM, 125 nM, 130 nM, 135 nM, 140 nM, 145 nM, 150 nM, 155 nM, 160 nM, 165 nM, 170 nM, 175 nM, 180 nM, 185 nM, 190 nM, 195 nM, 200 nM, 205 nM, 210 nM, 215 nM, 220 nM, 225 nM, 230 nM, 235 nM, 240 nM, 245 nM, 250 nM, 255 nM, 260 nM, 265 nM, 270 nM, 275 nM, 280 nM, 285 nM, 290 nM, 295 nM, 300 nM, 305 nM, 310 nM, 315 nM, 320 nM, 325 nM, 330 nM, 335 nM, 340 nM, 345 nM, 350 nM, 355 nM, 360 nM, 365 nM, 370 nM, 375 nM, 380 nM, 385 nM, 390 nM, 395 nM, 400 nM, 405 nM, 410 nM, 415 nM, 420 nM, 425 nM, 430 nM, 435 nM, 440 nM, 445 nM, 450 nM, 455 nM, 460 nM, 465 nM, 470 nM, 475 nM, 480 nM, 485 nM, 490 nM, 495 nM, 500 nM, 505 nM, 510 nM, 515 nM, 520 nM, 525 nM, 530 nM, 535 nM, 540 nM, 545 nM, 550 nM, 555 nM, 560 nM, 565 nM, 570 nM, 575 nM, 580 nM, 585 nM, 590 nM, 595 nM, 600 nM, 605 nM, 610 nM, 615 nM, 620 nM, 625 nM, 630 nM, 635 nM, 640 nM, 645 nM, 650 nM, 655 nM, 660 nM, 665 nM, 670 nM, 675 nM, 680 nM, 685 nM, 690 nM, 695 nM, 7000 nM, 705 nM, 710 nM, 715 nM, 720 nM, 725 nM, 730 nM, 735 nM, 740 nM, 745 nM, or 750 nM.

In some embodiments, the inhibitor inhibits GSK3β with an IC₅₀ of 100 nM or less. In some embodiments, the inhibitor inhibits GSK3β with an IC₅₀ about 5 to about 100 nM. In some embodiments, the inhibitor inhibits GSK3β with an IC₅₀ of about 5 nM. In some embodiments, the inhibitor inhibits GSK3β with an IC₅₀ of about 10 nM. In some embodiments, the inhibitor inhibits GSK3β with an IC₅₀ of about 15 nM. In some embodiments, the inhibitor inhibits GSK3β with an IC₅₀ of about 20 nM. In some embodiments, the inhibitor inhibits GSK3β with an IC₅₀ of about 25 nM. In some embodiments, the inhibitor inhibits GSK3β with an IC₅₀ of about 30 nM. In some embodiments, the inhibitor inhibits GSK3β with an IC₅₀ of about 35 nM. In some embodiments, the inhibitor inhibits GSK3β with an IC₅₀ of about 40 nM. In some embodiments, the inhibitor inhibits GSK3β with an IC₅₀ of about 45 nM. In some embodiments, the inhibitor inhibits GSK3β with an IC₅₀ of about 50 nM. In some embodiments, the inhibitor inhibits GSK3β with an IC₅₀ of about 55 nM. In some embodiments, the inhibitor inhibits GSK3β with an IC₅₀ of about 60 nM. In some embodiments, the inhibitor inhibits GSK3β with an IC₅₀ of about 65 nM. In some embodiments, the inhibitor inhibits GSK3β with an IC₅₀ of about 70 nM. In some embodiments, the inhibitor inhibits GSK3β with an IC₅₀ of about 75 nM. In some embodiments, the inhibitor inhibits GSK3β with an IC₅₀ of about 80 nM. In some embodiments, the inhibitor inhibits GSK3β with an IC₅₀ of about 85 nM. In some embodiments, the inhibitor inhibits GSK3β with an IC₅₀ of about 90 nM. In some embodiments, the inhibitor inhibits GSK3β with an IC₅₀ of about 95 nM. In some embodiments, the inhibitor inhibits GSK3β with an IC₅₀ of about 100 nM.

In some embodiments, the IC₅₀ can be calculated by using the Broad Repurposing Library and the PRISM multiplexed cell-line viability assay. Any assay or technique known in the art can be used for the calculation of the IC₅₀ for the purposed of the present invention.

### Methods of the Invention

In some embodiments, the present disclosure provides a method of treating a lymphocyte associated disease or condition, the method comprising administering to a subject in need thereof an effective amount of an agonist or activator of a β-catenin:Ikaros zinc finger (IKZF) protein complex.

In some embodiments, the present disclosure provides a method of eradicating pathogenic lymphocyte populations, the method comprising administering to a subject in need thereof a therapeutically effective amount of an agonist or activator of a β-catenin:IKZF protein complex.

In some embodiments, the present disclosure provides a method of enhancing adoptive cellular therapy (ACT) (e.g., via preconditioning) in a subject, the method comprising administering to a subject in need thereof a therapeutically effective amount of an agonist or activator of a β-catenin: IKZF protein complex. In certain embodiments, the agonist or activator of a β-catenin:Ikaros zinc finger (IKZF) protein complex is administered prior to administering the ACT.

In some embodiments, the present disclosure provides a method of treating a lymphopenic associated disease or condition, the method comprising administering to a subject in need thereof an effective amount of an agent that inhibits the expression or function of β-catenin or a β-catenin:IKZF protein complex. In certain embodiments, the β-catenin gene is knocked out or knocked down.

In some embodiments, the present disclosure provides a method of enhancing ACT in a subject, the method comprising administering to a subject in need thereof or an ACT preparation a therapeutically effective amount of an agent that inhibits the expression or function of β-catenin or a β-catenin:Ikaros zinc finger (IKZF) protein complex. In certain embodiments, the β-catenin gene is knocked out or knocked down.

In some embodiments, the IKZF protein can be IKZF1, IKZF2, or IKZF3. In some embodiments, the IKZF protein can be IKZF1. In some embodiments, the IKZF protein can be IKZF2. In some embodiments, the IKZF protein can be IKZF3. In some embodiments, the IKZF protein can be IKZF1, IKZF2, and IKZF3. In some embodiments, the IKZF protein can be IKZF1 and IKZF2. In some embodiments, the IKZF protein can be IKZF1 and IKZF3. In some embodiments, the IKZF protein can be IKZF2 and IKZF3. In some embodiments, the IKZF protein can be IKZF1 or IKZF2. In some embodiments, the IKZF protein can be IKZF1 or IKZF3. In some embodiments, the IKZF protein can be IKZF2 or IKZF3.

In some embodiments, the lymphocyte associated disease or condition can be a B-lymphoid malignancy and/or a T-lymphoid malignancy. In some embodiments, the lymphocyte associated disease or condition can be a B-lymphoid malignancy. In some embodiments, the lymphocyte associated disease or condition can be a T-lymphoid malignancy.

In some embodiments, the disease or condition can be a premalignant condition. In some embodiments, the premalignant condition can lead to overt leukemia or lymphoma. In some embodiments, the premalignant condition can be lymphoid clonal hematopoiesis of indeterminate potential (L-CHIP), Monoclonal B lymphocytosis (MBL), or a monoclonal gammopathy of unknown significance (MGUS).

In some embodiments, the lymphocyte associated disease or condition can be a cancer. In some embodiments, the cancer can be a myeloid cancer. In some embodiments, the cancer can be a B-cell cancer. In some embodiments, the cancer can be a T-cell cancer.

In some embodiments, the cancer can be an acute T-lymphoblastic lymphoma/leukemia (T-ALL). In some embodiments the cancer can be a peripheral T-cell lymphoma (PTCL). In some embodiments, the cancer can be cutaneous T-cell lymphomas, adult T-cell leukemia/lymphoma, angioimmunoblastic T-cell lymphoma, extranodal natural killer/T-cell lymphoma, enteropathy-associated intestinal T-cell lymphoma (EATL), anaplastic large cell lymphoma (ALCL), peripheral T-cell lymphoma not otherwise specified (PTCL-NOS).

In come embodiments, the cancer can be B-cell acute lymphoblastic leukemia (B-ALL), diffuse large B-cell lymphoma (DLBCL), follicular lymphoma, chronic lymphocytic leukemia (CLL)/small lymphocytic lymphoma (SLL), mantle cell lymphoma (MCL), marginal zone lymphoma, Burkitt lymphoma, lymphoplasmacytic lymphoma (Waldenstrom macroglobulinemia), hairy cell leukemia, primary central nervous system (CNS) lymphoma, primary intraocular lymphoma, non-Hodgkin lymphoma (NHL).

In some embodiments, the lymphocyte associated disease or condition can be an autoimmune disease. In some embodiments, the autoimmune disease can be driven by pathological autoreactive B- and T-lymphocyte populations. In some embodiments, the autoimmune disease can be arthritis, rheumatoid arthritis, systemic lupus erythematosus, vasculitis, scleroderma, or Sjogren disease.

In some embodiments, the lymphocyte associated disease or condition can be a graft versus host disease (GvHD).

In some embodiments, the lymphopenic associated disease or condition is lymphocytopenia and/or bone marrow failure.

In some embodiments, the lymphopenic associated disease or condition is caused by myeloid skewing, immunosenescence, side effects of drug-treatment, bone marrow transplantation, viral infections, and/or immunodeficiencies.

In some embodiments, the inhibitor of the β-catenin:IKZF protein complex can be administered in combination with at least one other treatment regimen for the lymphocyte associated disease or condition.

In some embodiments, the at least one other treatment comprising glucocorticoids; azathioprine; methotrexate; a combination of vincristine, prednisolone, L-asparaginase, daunorubicin (VPLD); a combination of cyclophosphamide, vincristine, Adriamycin, and dexamethasone (hyper-CVAD); a combination of rituximab, cyclophosphamide, doxorubicin, vincristine, and prednisone (R-CHOP); a combination of cyclophosphamide, doxorubicin, vincristine, and prednisone (CHOP); or combinations thereof.

In some embodiments, the GSK3β inhibitors of the present disclosure can be used in combination with standard treatment for autoimmune diseases and lymphoid malignancies below:

| Autoimmune Disease or | |
|---|---|
| Lymphoid Malignancy | Standard Treatment |
| SLE | glucocorticoids, azathioprine, methotrexate |
| Rheumatoid arthritis | methotrexate |
| B- and T-ALL in | vincristine, prednisolone, L-asparaginase, |
| children | daunorubicin (VPLD) |
| B- and T-ALL in | cyclophosphamide, vincristine, Adriamycin, |
| adults | and dexamethasone (hyper-CVAD) |
| B-cell lymphoma | rituximab, cyclophosphamide, doxorubicin, |
| | vincristine, and prednisone (R-CHOP) |
| T-cell lymphoma | cyclophosphamide, doxorubicin, vincristine, |
| | and prednisone (CHOP) |

In some embodiments, the disease or condition can be a drug-resistant disease or condition.

In some embodiments the inhibitor can be administered by a common route of entry. In some embodiments, the inhibitor can be administered intravenously, subcutaneously, orally, or intranasally. In some embodiments, the inhibitor can be administered orally. Non-limiting examples of routes of entry by which the inhibitor may be administered include orally, intravenously, transdermally, by inhalation, or rectally. In some embodiments, the inhibitors can be formulated for parenteral administration, e.g., intravascular (intravenous or intraarterial), intraperitoneal, intratumoral, intraventricular, intrapleural or intramuscular administration. In some embodiments, the inhibitor can be reconstituted from a lyophilized preparation prior to administration.

It is also contemplated that when used to treat various diseases/disorders, the methods and inhibitors of the present disclosure can be utilized with additional therapeutic methods/agents suitable for the same or similar diseases/disorders. In certain embodiments, such other therapeutic methods/agents can be co-administered (simultaneously or sequentially) to generate additive or synergistic effects. Suitable therapeutically effective dosages for each agent may be lowered due to the additive action or synergy.

In some embodiments, the methods and/or inhibitors of the present disclosure can be used in combination with at least one additional cancer therapy. For example, the methods and/or inhibitors of the present disclosure can be used in combination with conventional cancer therapies, such as, e.g., surgery, chemotherapy or combinations thereof, depending on type of the tumor, patient condition, other health issues, and a variety of factors. Non-limiting examples of cancer therapies also include radiation therapy, bone marrow transplant, immunotherapy, hormone therapy, targeted drug therapy, cryoablation, and radiofrequency ablation. In some embodiments, the additional cancer therapy includes administering to a subject at least one chemotherapeutic agent that is not a β-catenin.IKZF protein complex inhibitor. In some embodiments, the radiation is X-rays, gamma rays, alpha particles, beta particles, proton beams, neutron beams, or negative Pi mesons.

In some embodiments, the GSK3β inhibitor can be administered at a dose range from 5 nM to 500 nM. In some embodiments, the inhibitor is administered at a dose of about 0.1 nM to about 900 nM, about 1 nM to about 750 nM, about 5 nM to about 500 nM, about 10 nM to about 400 nM, about 20 nM to about 300 nM, about 30 nM to about 250 nM, about 40 nM to about 200 nM, or about 50 nM to about 100 nM. In some embodiments, the GSK3β inhibitor can be administered to the subject a dose sufficient to result in a steady state plasma concentration of the inhibitor from about 1 nM to about 100 nM, from about 1 nM to about 200 nM, from about 1 nM to about 300 nM, from about 1 nM to about 400 nM, from about 1 nM to about 500 nM, 5 nM to about 100 nM, from about 5 nM to about 200 nM, from about 5 nM to about 300 nM, from about 5 nM to about 400 nM, from about 5 nM to about 500 nM about 10 nM to about 100 nM, from about 10 nM to about 200 nM, from about 10 nM to about 300 nM, from about 10 nM to about 400 nM, or from about 10 nM to about 500 nM. In some embodiments, the GSK3β inhibitor is administered to the subject a dose sufficient to result in a steady state plasma concentration of the inhibitor of about, at least about, or no more than about 0.1 nM, 0.2 mM, 0.3 nM, 0.4 nM, 0.5 nM, 0.6 nM, 0.7 mM, 0.8 nM, 0.9 nM, 1 nM, 1.5 nM, 2 nM, 2.5 nM, 3 nM, 3.5 nM, 4 nM, 4.5 nM 5 nM, 10 nM, 15 nM, 20 nM, 25 nM, 30 nM, 35 nM, 40 nM, 45 nM, 50 nM, 55 nM, 60 nM, 65 nM, 70 nM, 75 nM, 80 nM, 85 nM, 90 nM, 95 nM, 100 nM, 105 nM, 110 nM, 115 nM, 120 nM, 125 nM, 130 nM, 135 nM, 140 nM, 145 nM, 150 nM, 155 nM, 160 nM, 165 nM, 170 nM, 175 nM, 180 nM, 185 nM, 190 nM, 195 nM, 200 nM, 205 nM, 210 nM, 215 nM, 220 nM, 225 nM, 230 nM, 235 nM, 240 nM, 245 nM, 250 nM, 255 nM, 260 nM, 265 nM, 270 nM, 275 nM, 280 nM, 285 nM, 290 nM, 295 nM, 300 nM, 305 nM, 310 nM, 315 nM, 320 nM, 325 nM, 330 nM, 335 nM, 340 nM, 345 nM, 350 nM, 355 nM, 360 nM, 365 nM, 370 nM, 375 nM, 380 nM, 385 nM, 390 nM, 395 nM, 400 nM, 405 nM, 410 nM, 415 nM, 420 nM, 425 nM, 430 nM, 435 nM, 440 nM, 445 nM, 450 nM, 455 nM, 460 nM, 465 nM, 470 nM, 475 nM, 480 nM, 485 nM, 490 nM, 495 nM, 500 nM, 505 nM, 510 nM, 515 nM, 520 nM, 525 nM, 530 nM, 535 nM, 540 nM, 545 nM, 550 nM, 555 nM, 560 nM, 565 nM, 570 nM, 575 nM, 580 nM, 585 nM, 590 nM, 595 nM, 600 nM, 605 nM, 610 nM, 615 nM, 620 nM, 625 nM, 630 nM, 635 nM, 640 nM, 645 nM, 650 nM, 655 nM, 660 nM, 665 nM, 670 nM, 675 nM, 680 nM, 685 nM, 690 nM, 695 nM, 7000 nM, 705 nM, 710 nM, 715 nM, 720 nM, 725 nM, 730 nM, 735 nM, 740 nM, 745 nM, or 750 nM.

In some embodiments, the inhibitor inhibits GSK3β with an IC₅₀ of 100 nM or less. In some embodiments, the inhibitor inhibits GSK3β with an IC₅₀ about 5 to about 100 nM. In some embodiments, the inhibitor inhibits GSK3β with an IC₅₀ of about 5 nM. In some embodiments, the inhibitor inhibits GSK3β with an IC₅₀ of about 10 nM. In some embodiments, the inhibitor inhibits GSK3β with an IC₅₀ of about 15 nM. In some embodiments, the inhibitor inhibits GSK3β with an IC₅₀ of about 20 nM. In some embodiments, the inhibitor inhibits GSK3β with an IC₅₀ of about 25 nM. In some embodiments, the inhibitor inhibits GSK3β with an IC₅₀ of about 30 nM. In some embodiments, the inhibitor inhibits GSK3β with an IC₅₀ of about 35 nM. In some embodiments, the inhibitor inhibits GSK3β with an IC₅₀ of about 40 nM. In some embodiments, the inhibitor inhibits GSK3β with an IC₅₀ of about 45 nM. In some embodiments, the inhibitor inhibits GSK3β with an IC₅₀ of about 50 nM. In some embodiments, the inhibitor inhibits GSK3β with an IC₅₀ of about 55 nM. In some embodiments, the inhibitor inhibits GSK3β with an IC₅₀ of about 60 nM. In some embodiments, the inhibitor inhibits GSK3β with an IC₅₀ of about 65 nM. In some embodiments, the inhibitor inhibits GSK3β with an IC₅₀ of about 70 nM. In some embodiments, the inhibitor inhibits GSK3β with an IC₅₀ of about 75 nM. In some embodiments, the inhibitor inhibits GSK3β with an IC₅₀ of about 80 nM. In some embodiments, the inhibitor inhibits GSK3β with an IC₅₀ of about 85 nM. In some embodiments, the inhibitor inhibits GSK3β with an IC₅₀ of about 90 nM. In some embodiments, the inhibitor inhibits GSK3β with an IC₅₀ of about 95 nM. In some embodiments, the inhibitor inhibits GSK3β with an IC₅₀ of about 100 nM.

In some embodiments, the pathogenic lymphocyte can be a drug-resistant pathogenic lymphocyte.

In current embodiments, the composition of the present invention comprises the agent in immediate release, immediate release, controlled release, extended release, other release dosage form or pattern, or combinations thereof.

Excipients suitable for inclusion in compositions of the present disclosure include diluents, binders, disintegrants, dispersants, lubricants, glidants, stabilizers, interfaces. An activator and a colorant are included. Diluents, also called “fillers,” can be used to increase tablet bulk so that a practical size for tableting is obtained. Non-limiting examples of diluents include lactose, cellulose, microcrystalline cellulose, mannitol, dry starch, hydrolyzed starch, powdered sugar, talc, Sodium chloride, silicon dioxide, titanium oxide, dicalcium phosphate dihydrate, calcium sulfate, calcium carbonate (calcium calcium) alumina, and kaolin). A binder can impart tackiness to the tablet formulation, and the binder can be used to help keep a tablet intact after tableting. Non-limiting examples of suitable binders include starch (including corn starch and pregelatinized starch), gelatin, sugars (e.g., glucose, dextrose, sucrose, lactose, sorbitol, cellulose, polyethylene glycol, wax, natural rubber and synthetic rubber (e.g., natural and synthetic gums), acacia, tragacanth, sodium alginate, and synthetic polymers (polymethacrylates, polyvinylpyrrolidone, etc.). Non-limiting examples of lubricants include magnesium stearate, calcium stearate, stearic acid, glyceryl behenate, and polyethylene glycol. Disintegrants can facilitate tablet disintegration after administration, and non-limiting examples thereof include starch, alginic acid, cross-linked polymers (e.g., cross-linked polyvinyl pyrrolidone), croscarmellose sodium, glycol potassium acid starch, potassium or sodium starch glycolate, clay, cellulose, starch, gum, and combinations thereof. Non-limiting examples of suitable glidants include silicon dioxide and talc. Stabilizers can inhibit or delay drug degradation reactions (including oxidation reactions). Surfactants can also include and can be anionic, cationic, amphoteric, or nonionic surfactants. If desired, tablets may also contain non-toxic adjuvants (pH buffering agents, preservatives (e.g., antioxidants), wetting agents or emulsifying agents). Compositions of the present disclosure may further include solubilizing agents, coating agents, and/or flavoring agents.

Controlled release formulations can include one or more combinations of excipients that delay the release of the drug by coating the active drug or by transient binding or by reducing its solubility. Examples of these excipients include cellulose ethers (such as hydroxypropyl methylcellulose or silicified microcrystalline cellulose), polyvinyl acetate-based excipients, and methacrylate and methacrylic acid-based polymers and copolymers. In some embodiments, a composition is formulated for extended or controlled release.

Immediate release formulations include one or more combinations of excipients capable of rapid release (such as 1 minute to 1 hour after administration) of a pharmaceutically active agent (such as the pyrimidine synthesis inhibitor or the DNA repair inhibitor). In one embodiment, the immediate release excipient is microcrystalline cellulose, sodium carboxymethyl cellulose, sodium starch glycolate, corn starch, colloidal silica, sodium lauryl sulfate, magnesium stearate, croscarmellose sodium, crospovidone NF, Avicel PH200, and combinations thereof.

Pharmaceutical carriers or vehicles suitable for administration of compositions provided herein include all such carriers known to those skilled in the art to be appropriate for a particular mode of administration.

Compositions disclosed herein may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions may further comprise one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In accordance with the present invention there may be numerous tools and techniques within the skill of the art, such as those commonly used in molecular biology, pharmacology, and microbiology. Such tools and techniques are described in detail in e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

### Examples

The following examples are provided to further describe some of the embodiments disclosed herein. The examples are intended to illustrate, not to limit, the disclosed embodiments.

### Materials and Methods

### Primary Human Samples and Cell Lines

Patient samples (Table 1) were obtained in compliance with the Institutional Review Boards of University of California San Francisco and Yale University. Patient-derived primary human pre-B ALL xenografts were cultured in Alpha Minimum Essential Medium (MEMα; Life Technologies) with GlutaMAX containing 20% fetal bovine serum (FBS, Gibco), 100 IU ml⁻¹ penicillin/streptomycin (P/S; Gibco). Primary hematopoietic stem cells from cord blood were purchased from All Cells and cultured in StemSpan SFEM II medium (Stem Cell Technologies) supplemented with 1% P/S, 50 ng ml⁻¹ recombinant human thrombopoietin (TPO, Peprotech), 50 ng ml⁻¹ recombinant human stem cell factor (SCF, Peprotech), and 100 ng ml⁻¹ recombinant human angiopoietin-like protein 5 (Angpt15, Miltenyi). The human cell lines (Table 2) were cultured in RPMI-1640 (Gibco) with GlutaMAX containing 10% FBS, 100 IU ml⁻¹ P/S at 37° C. in a humidified incubator with 5% CO₂. All human primary samples and cell lines were tested negative for mycoplasma by detection kit (MycoAlert PLUS, LONZA).

TABLE 1
Overview of leukemia xenografts expanded in NSG mice

| | | | Disease | Cytogenetics/ | Gender/ |
|---|---|---|---|---|---|
| Case | Leukemia | Lesion | Stage | Karyotype | Age |
| BLQ5 | B-ALL | BCR-ABL1 | Relapse | FISH der(9), der(22) | f |
| ICN1 | B-ALL | BCR-ABL1 | Diagnosis | t(9; 22)(q34; q11) | m/14 |
| IAH8R | B-ALL | BCR-ABL1 | Relapse | t(9; 22)(q34; q11) | |
| JFK125R | B-ALL | P2RY8-CRLF2 | Relapse | +8, +21, +22 | m/49 |
| | | FLT3-ITD | | | |
| LAX2 | B-ALL | BCR-ABL1 | Relapse | t(9; 22)(q34; q11) | m/38 |
| LAX7 | B-ALL | IL7R^{T244}^{_{—}}^{I245Ins} | Diagnosis | 46, XY | m |
| LAX7R | B-ALL | KRAS^{G12V} | Relapse | 46, XY | m/12 |
| MXP2 | B-ALL | BCR-ABL1 | Diagnosis | t(9; 22)(q34; q11) | m |
| MXP5 | B-ALL | BCR-ABL1 | Diagnosis | t(9; 22)(q34; q11) | m/5 |
| PDX2 | B-ALL | BCR-ABL1 | Diagnosis | t(9; 22)(q34; q11) | f/52 |
| SFO5 | B-ALL | N/A | Diagnosis | N/A | m/23 |

TABLE 2
Overview of cell lines used in this study

| Cell line | Malignancy | Transforming lesions | Source |
|---|---|---|---|
| BV-173 | B-ALL | BCR-ABL1 | DMSZ |
| KASUMI-1 | AML | AML1-ETO; KIT^{N822K}; TP53^{R248G}; RAD21^{K330Pfs*6} | ATCC |
| KOPN8 | B-ALL | MLL-ENL; KRAS^{G12}; TP53^{R248G} | ATCC |
| NALM1 | B-ALL | BCR-ABL1 | ATCC |
| RCH-ACV | B-ALL | E2A-PBX1; EGFR^{R677C}; HRAS^{A11T}; | DSMZ |
| | | KRAS ^{V141} | |
| SEM | B-ALL | MLL-AF4; TP53^{R248G}; CDKN2A^{H83Y} | ATCC |
| SUP-B15 | B-ALL | BCR-ABL1 | DMSZ |
| TOM1 | B-ALL | BCR-ABL1 | DMSZ |
| HBL1 | ABC-DLBCL | N/A | ATCC |
| Karpas-422 | ABC-DLBCL | ARID1A ^{Q2176fs*48}; TP53 ^{K319*} | Sigma-Aldrich |
| SU-DHL-2 | ABC-DLBCL | TNFAIP3^{5459*}; MYD88^{S222R}; EP300^{Q821*} | Dr. Vu Ngo |
| OCI-LY7 | GCB-DLBCL | IGH-MYC; BCL6^{A587D; A531D} | DSMZ |
| OCI-LY19 | GCB-DLBCL | NRAS^{G61K} | DSMZ |
| SU-DHL4 | GCB-DLBCL | IGH-BCL2; EZH2^{Y646S; Y666N}; TP53^{R273C} | Dr. Vu Ngo |
| JEKO1 | MCL | IGH-CCND1; TP53-del | ATCC |
| MINO | MCL | IGH-CCND1; TP53^{V1476}; NRAS^{G13D}; CDKN2A^{Q88K} | Dr. Vu Ngo |
| Z-138 | MCL | IGH-CCND1; TRAF2 ^{W114*} | Dr. Vu Ngo |
| GUMBUS | BL | IGH-MYC | DSMZ |
| RAJI | BL | IGH-MYC; TP53^{R213Q; Y234H} | ATCC |
| RAMOS | BL | IGH-MYC; TP53^{I254D} | ATCC |
| KM-H2 | HL | PRRC2B-MGMT;CIITA-C15orf65 | DSMZ |
| L428 | HL | TP53^{G112-V122del11} | DSMZ |
| JJN3 | MM | NRAS^{G61K} | DSMZ |
| LP-1 | MM | TP53^{G286K}; IGH-NSD2; FGFR3^{P384L}; TRAF3 ^{K2861} | DSMZ |
| U266 | MM | BRAF^{K601N}; TP53^{A161T}; TRAF3^{K550Lfs*}; MSH6^{G141A} | ATTC |
| JURKAT | T-ALL | TP53^{R196*}; NOTCH1^{R1627H}, FBXW7^{R505C} | ATCC |
| KOPTK-1 | T-ALL | NOTCH1 \| CDKN2A \| CREBBP | David Weinstock, |
| | | | DF/HCC |
| MOLT3 | T-ALL | NRAS^{Q61K}; NOTCH1^{L1600P}; PTEN^{K267Rfs*9} | ATCC |
| FE-PD | CTCL | JAK1^{G1097V}; STAT3^{G618R} | David Weinstock, |
| | | | DF/HCC |
| HH | CTCL | FOXK2-TP63; TP53^{Y126-K132del} | David Weinstock, |
| | | | DF/HCC |
| HUT-78 | CTCL | NRAS^{G61K}; TP53^{R196*} | David Weinstock, |
| | | | DF/HCC |
| H9 | CTCL | NRAS^{G61K}; TP53^{R196*} | David Weinstock, |
| | | | DF/HCC |
| MAC1 | CTCL | PCM1-JAK2 | ATCC |
| MA9.3 | AML | MLL-AF9; FLT3-ITD | Dr. Jianjun Chen |
| MV-4-11 | AML | KMT2A-AFF1; FLT3-ITD | ATCC |
| MOLM13 | AML | MLL-AF9 | DSMZ |
| MONOMAC6 | AML | MLL-AF9; TP53^{R273H}; RUNX1-ATP8A2 | DSMZ |
| THP1 | AML | MLL-AF9; CSNK2A1-DDX39B; NRAS^{G12D}; TP53^{R174del} | ATCC |
| JURL-MK1 | CML | BCR-ABL1; KMT2D^{Y1495*}; TP53^{R306*} | DSMZ |
| KCL22 | CML | BCR-ABL1; MSH6^{F1088Sfs*2};TP53^{P301Gfs*44} | DSMZ |
| HT-29 | Colon carcinoma | TP53^{R273H}; APC^{D853*}; BRAF^{V600E}; SMAD4^{Q311*}; | ATCC |
| | | PIK3CA^{P449T} | |
| HCT116 | Colon carcinoma | KRAS^{G13D}; CTNNB1^{S45del}; BRCA2^{I2675Dfs*6}; | ATCC |
| | | CDKN2A^{R24Sfs*20}; ^{E33Rfs*20;D74fs*21} | |
| SW620 | Colon carcinoma | APC^{Q1338*};*; KRAS^{G12V}; TP53^{R273H; P309S} | Dr. Mingye Feng |
| SW480 | Colon carcinoma | APC^{Q1338*}; KRAS^{612V}; TP53^{R273H; P309S} | ATCC |
| LOVO | Colon carcinoma | APC ^{R1114*}; FBXW^{7R505C}; KRAS^{G13D} | ATCC |
| DLD1 | Colon carcinoma | APC^{R2166*}; EP300^{Q1014*}; KRAS^{G13D}; TP53^{S241P}; | Dr. Mingye Feng |
| | | PIK3CA^{G545K; D549N} | |
| H244 | NSCLC | KRAS^{G12V}; TP53^{Y236C} | ATCC |
| H249 | SCLC | PIK3CA^{G106-R108del}, RB1^{E748*}; TP53^{E171*} | ATCC |
| H82 | SCLC | PVT1-MYH7 | ATCC |
| H146 | SCLC | KMT2D^{D2911*}; RB1^{G850*}; TP53^{P318Lfs*21} | ATCC |
| H524 | SCLC | RB1^{S576*}; ATM^{R3375}; TP53^{T155N} | ATCC |
| H446 | SCLC | PVT1-LY6H; TP53^{G154V} | ATCC |
| H526 | SCLC | TP53 splicing mutation | ATCC |
| M285 | Melanoma | N/A | DSMZ |
| M230 | Melanoma | KIT ^{L576P} | DSMZ |
| M229 | Melanoma | BRAF ^{V600Q}; PTEN-del | DSMZ |

### Genetic Mouse Models

NSG, NSGW41 and Axin2-TQ mice were purchased from Jackson Laboratory. Ctnnb1^{ex3fl} strain was provided by Mark Taketo. To activate β-catenin in B cell precursors Ctnnb1^{ex3fl} mice were crossed to Mb1-cre mice. Mice homozygous or heterozygous for the Ctnnb1^{ex3fl} locus were used and since no significant differences were observed between homozygous and heterozygous mice, the exact genotype is not indicated. Both Cre-positive and negative animals were used as controls and no significant differences were observed between these two types of control animals. For modelling B-ALL transformation mice heterozygous for the Ctnnb1^{ex3fl} locus were used. All animals were maintained in a specific pathogen free environment. Experiments were approved by the regional council in Freiburg, Germany and Yale University, USA and carried out in accordance with the German Animal Welfare Act and Institutional Animal Care & Use Committee. Genetic mouse models used in this study are listed in Table 3. The genotyping primers used in this study are shown in Table 4.

TABLE 3
Overview of genetic mouse models used in this study

| Mouse strain | Investigator | Purpose |
|---|---|---|
| Axin2-mTurqouise | Frank Staal, Leiden University | Wnt/β-catenin reporter mice |
| (Axin2-TQ) | Medical Center | |
| Ctnnb1^{ex3fl} | Mark M. Taketo, Kyoto University | Genetic gain-of function experiments |
| Mb1^{cre} Ctnnb1^{ex3fl} | Jellusova Lab, TUM | B-cell specific β-catenin activation |
| NOD.Cg-Prkdc^{scid} | Leonard D. Shultz | Transplant recipient mice |
| Il2rg^{tm1Wjl}/SzJ (NSG) | The Jackson Laboratory | |
| NOD.Cg^{KitW-41J} Tyr⁺ Prkdc^{scid} | James Thomson, | Transplant recipient mice |
| Il2rg^{tm1Wjl}/SzJ/ThomJ | The University of Wisconsin | |
| (NSGW41) | | |

TABLE 4
Oligonucleotide sequences of primers and gRNA sequences for CRISPR-mediated
gene modification

| Experiment | Name | Sequence (5′ to 3′) | SEQ ID NO: |
|---|---|---|---|
| Axin2-TQ mouse genotyping | Forward | CGATTGCTGAGAGGAACTGG | 8 |
| Axin2-TQ mouse genotyping | WT Reverse | AGTGTAAAGACTTGGTCCACCTG | 9 |
| Axin2-TQ mouse genotyping | Mutant Reverse | CAGGACAGGGTGGTCACG | 10 |
| Ctnnb1^{ex3fl} mouse genotyping | Forward | AACTGGCTTTTGGTGTCGGG | 11 |
| Ctnnb1^{ex3fl} mouse genotyping | Reverse | TCGGTGGCTTGCTGATTATTTC | 12 |
| Mb1^{cre} mouse genotyping | WT Forward | TTCAGCCTTCAGTCTAACATC | 13 |
| Mb1^{cre} mouse genotyping | WT Reverse | ATCTGTGAAGACAGGGTG | 14 |
| Mb1^{cre} mouse genotyping | Mutant Forward | CCCTGTGGATGCCACCTCC | 15 |
| Mb1^{cre} mouse genotyping | Mutant Reverse | GTCCTGGCATCTGTCAGAG | 16 |
| Non-targeting control | gRNA control | GCGAGGTATTCGGCTCCGCG | 1 |
| Mouse Ctnnb1 deletion | gRNA target | ACAATGGCAGACACCATCTG | 2 |
| Mouse Ikzf1 deletion | gRNA target | CTGGAGTGTCACTGACTGGG | 3 |
| Mouse Ikzf3 deletion | gRNA target | ATTATGAAGCCGGAGCCCAT | 4 |
| Non-targeting control | gRNA control | GGTTCTTGACTACCGTAATT | 5 |
| Human CTNNB1 deletion | gRNA target | AAGGTTATGCAAGGTCCCAG | 6 |

### Murine Primary and Leukemia Cells

Bone marrow cells were harvested from 6-12 weeks old mice by flushing cavities of femur and tibia with ice-cold PBS supplemented with 2% FBS and cells were filtered through 70 m mesh to generate single cell suspensions. Cells from spleen and thymus were extracted by forcing tissues through a 40 m strainer into ice-cold PBS with 2% FBS. Erythrocyte lysis was performed for bone marrow and spleen cells (RBC Lysis Buffer, BioLegend). After making single cell suspensions, bone marrow cells were cultured in Iscove's modified Dulbecco's medium (IMDM; Gibco) with GlutaMAX containing 20% FBS, 50 mol ml⁻¹ 2-mercaptoethanol, 100 IU ml⁻¹ P/S. Bone marrow cells were cultured in 10 ng ml⁻¹ recombinant mouse IL-7 (Peprotech) to generate IL-7 dependent pre-B cells. For BCR-ABL1 driven leukemia model, pre-B cells were retrovirally transduced by BCR-ABL1 (Table 5) and IL-7 was removed to promote the outgrowth of the transformed cells. For NRAS^{G12D} leukemia model, pre-B cells were retrovirally transduced by NRAS^{G12D} (Table 5) and cultured in the presence of IL-7. Lineage-depleted cells (Gr-1, CD11 b, CD3e, CD49b, Ter 19 and B220 negative) were cultured in 10ng ml⁻¹ recombinant mouse IL-7 (Sigma), 50 ng ml⁻¹ recombinant mouse FLT3L (Sigma) and 50 ng ml⁻¹ recombinant mouse SCF (Sigma) in Opti-MEM medium (Gibco) supplemented with 20% FBS premium (PAN), 1 mmol 1-1 sodium pyruvate (Thermo Fisher Scientific), 2 mmol 1-1 Glutamax, 25 mmol 1-1 HEPES, 1% P/S, 57 μmol⁻¹ 2-mercaptoethanol (Sigma) at 37° C. in an atmosphere with 7.5% CO2. FLT3L was withdrawn after 3 days and SCF was withdrawn after 6-8 days to induce pre-B cell differentiation. Lineage negative mouse hematopoietic progenitors from bone marrow were cultured in Ham's F-12 Nutrient Mix liquid medium (Gibco) supplemented with 10 mmol 1⁻¹ HEPES, 100 ng ml⁻¹ TPO, 10 ng ml⁻¹ SCF, lx Insulin-transferrin-selenium-ethanolamine (Gibco), 1× Penicillin-streptomycin-glutamine (Gibco) and 1 Ig ml⁻¹ Polyvinyl alcohol (Sigma) on fibronectin coated plates (Corning).

TABLE 5
Retroviral and lentiviral constructs

| Construct | Expression of | Source |
|---|---|---|
| FUCas9mCherry | Tet-On; Cas9 | Addgene |
| H1-gRNA-TetR-TagBFP | gRNA; BFP | Müschen Laboratory, Yale University |
| MSCV-BCR-ABL1 | BCR-ABL1 | Müschen Laboratory, Yale University |
| MSCV-NRAS^{G12D}-Puro | NRAS^{G12D} | Müschen Laboratory, Yale University |
| MSCV ER^{T2}-Puro | ER^{T2}; Puromycin resistance | Müschen Laboratory, Yale University |
| MSCV Cre-ER^{T2}-Puro | Cre-ER^{T2}; | Müschen Laboratory, Yale University |
| | Puromycin resistance | |
| MSCV ER^{T2}-GFP | ER^{T2}; GFP | Müschen Laboratory, Yale University |
| MSCV Cre-ER^{T2}-GFP | Cre-ER^{T2}; GFP | Müschen Laboratory, Yale University |
| MSCV-EV-RFP | RFP | Müschen Laboratory, Yale University |
| MSCV-FOXP1-RFP | FOXP1; RFP | Müschen Laboratory, Yale University |
| MSCV-EV-GFP | MYC; GFP | Müschen Laboratory, Yale University |
| MSCV-MycT58A-GFP | MYC^{T58A}; GFP | Müschen Laboratory, Yale University |
| MSCV-Myc-GFP | MYC; GFP | Müschen Laboratory, Yale University |
| pCL6-Luc-Blasticidin | Firefly luciferase | Müschen Laboratory, Yale University |
| pLKO hPGK-puro | sh-Ctnnb1(TRCN0000012692) | Sigma Aldrich |
| pLKO hPGK-puro | scrambled | Sigma Aldrich |
| pLVX-Tre-3G- | GFP; puromycin resistance | Müschen Laboratory, Yale University |
| EV-ires-GFP-puro | | |
| pLVX-Tre-3G- | IKZF1; GFP; puromycin | Müschen Laboratory, Yale University |
| IKZF1-ires-GFP-puro | resistance | |
| pLVX-Tre-3G- | GFP; puromycin resistance | Müschen Laboratory, Yale University |
| EV-ires-GFP-puro | | |
| pLVX-Tre-3G- | CTNNB1^{S33A/S37A/T41A/S45A}; GFP; | Müschen Laboratory, Yale University |
| CTNNB1^{S33A/S37A/T41A/S45A}- | puromycin resistance | |
| ires-GFP-puro | | |
| pLVX-Tet3G-Neo | Tet-On | Clontech Laboratories |
| pRetroX-Tet3G-Neo | Tet-On | Clontech Laboratories |
| pRetroX-Tre-3-G-EV-puro | Tet-On | Muschen Laboratory, Yale University |
| pRetroX-Tre-3-G-Cebpa-puro | Tet-On | Muschen Laboratory, Yale University |

### Retroviral and Lentiviral Transduction

Vectors used for retroviral or lentiviral transduction are listed in Table 5. For virus production 70% confluent HEK 293FT cells were transfected with Lipofectamine 2000 (Invitrogen) reagent according to manufacturer's instructions and cultured in high glucose Dulbecco's modified Eagle's medium (DMEM; Gibco) with GlutaMAX containing 10% FBS, 100 IU ml⁻¹ P/S (Gibco), 1 mmol 1⁻¹ sodium pyruvate (Gibco) and 0.1 mmol 1⁻¹ non-essential amino acids (Gibco). For retrovirus production, pHIT60 (gag-pol) and pHIT123 vectors were used whereas, pCDNL/BH and EM140 or VSVG vectors were used for lentivirus generation. One day after transfection, virus production was induced by treating the cells with 10 mmol 1⁻¹ Sodium butyrate (Sigma-Aldrich) for 6-8 hours. 24 hours after medium change, the virus containing supernatants were collected and filtered through a 0.45 μm filter. For retroviral transduction: viral supernatants were loaded by centrifugation (2,000 g, 90 min at 32° C.) on 50 μg ml⁻¹ Retronectin (Takara) coated non-tissue culture 6-well plates. 2-3 million cells were transduced per well by centrifugation at 600 g for 30 min in the appropriate culture medium and maintained at 37° C. at 5% CO₂ for 48 h. For lentiviral transduction: 2-4 million cells per well were centrifuged at 600 g for 30 min in the presence of lentiviral supernatant (concentrated by ultra-centrifugation) and maintained at 37° C. at 5% CO₂. The lentiviral supernatants were replaced with fresh medium 16 hours after transduction.

### Western Blotting

Cells were washed twice with ice-cold PBS and were lysed in CelLytic buffer (Sigma-Aldrich) supplemented with 1% protease inhibitor cocktail (Roche Diagnostics), 1% phosphatase inhibitor cocktail (EMD Millipore) and 1 mM PMSF (CST) on ice. 10-20 μg of cell lysates were separated on precast gels (Bio-Rad) and transferred on nitrocellulose membranes (Bio-Rad). After blocking for an hour in TBS-T with 2% BSA, membranes were probed with the appropriate primary antibodies listed in Table 6. Membranes were incubated with alkaline-phosphatase conjugated secondary antibodies (Invitrogen) and analyzed with Chemi Doc™ MP Imaging System (Bio-Rad). For fractionation experiment Nuclear and Cytoplasmic Extraction Reagent (Thermo Scientific) was used according to manufacturer's instructions.

TABLE 6
Western blot antibodies used in this study

| Antigen | Clone | Manufacturer |
|---|---|---|
| Axin1 | C76H11 | Cell Signaling Technology |
| β-actin | SC-47778 | Santa Cruz Biotech |
| β-catenin | 14/Beta-Catenin | BD Biosciences |
| β-tubulin | D2N5G | Cell Signaling Technology |
| C/ebp α1 | D56F10 | Cell Signaling Technology |
| Chd (Mi-2β) | 72418 | Abcam |
| Dagkα | 11547-1-AP | Proteintech |
| Foxp1 | D35D10 | Cell Signaling Technology |
| Ikzf1 | D6N9Y | Cell Signaling Technology |
| Ikzf2 | D8W4X | Cell Signaling Technology |
| Ikzf3 | D1C1E | Cell Signaling Technology |
| Lef1 | C12A5 | Cell Signaling Technology |
| Mta2 | 8106 | Abcam |
| Myc | D84C12 | Cell Signaling Technology |
| Myc-pT⁵⁸ | PA5-36673 | Invitrogen |
| Myc-pS⁶² | E1J4K | Cell Signaling Technology |
| Prdm1 | C14A4 | Cell Signaling Technology |
| Tbp | D5C9H | Cell Signaling Technology |
| Tcf1/Tcf7 | C63D9 | Cell Signaling Technology |
| Tcf4/Tcf712 | C48H11 | Cell Signaling Technology |
| Tead1 | D9X2L | Cell Signaling Technology |

### Flow Cytometry

Cells were washed twice with PBS containing 2% FBS and blocked with Fc blocker (BD Biosciences) for 20 min on ice. Cells were stained with the appropriate antibodies listed in Table 7 or isotype controls for 30 min on ice. Cells were then washed and resuspended in PBS containing 0.75 g ml⁻¹ of DAPI to exclude dead cells and analyzed on LSRFortessa X-20 or FACSSympony A3 flow cytometer (BD Biosciences). FACSAria III or FACSAria Fusion (BD Biosciences) were used for fluorescence based cell sorting experiments. For apoptosis analysis, annexin V (BioLegend) and DAPI (BioLegend) reagents were used according to the manufacturer's instructions. For cell cycle analysis, Click-iT EdU kit (Invitrogen) was used according to manufacturer's instructions. All the FACS data were analyzed using FlowJo software (FlowJo, LLC).

TABLE 7
Flow cytometry antibodies used in this study

| Antigen | Clone | Manufacturer |
|---|---|---|
| B220 (CD45R) | RA3-6B2 | Biolegend |
| BP-1 (Ly-51) | 6C3 | Biolegend |
| Ccr2 | 475301 | R&D Systems |
| Cd19 | 6D5 | Biolegend |
| Cd21/CD35 | 7G6 | BD Biosciences |
| Cd23 | B3B4 | BD Biosciences |
| Cd24 | M1/69 | Biolegend |
| Cd244.2 | 2B4 | BD Biosciences |
| Cd25 | PC61 | Biolegend |
| Cd4 | RM4-5 | Biolegend |
| Cd43 | S11 | Biolegend |
| Cd45 | 30F11 | Biolegend |
| Cd5 | 53-7.3 | BD Biosciences |
| Cd8 | 53-6.7 | Biolegend |
| Ctla4 | UC10-4B9 | Biolegend |
| Gr-1 (Ly-6G/Ly-6C) | RB6-8C5 | Biolegend |
| IgD | 11-26c.2a | Biolegend |
| IgM | R6-60.2 | Biolegend |
| Nk-1.1 | PK136 | Biolegend |
| Ter119 | TER-119 | Biolegend |

### Cell Viability Assay

Forty thousand patient-derived pre-B ALL cells or twenty thousand leukemia/lymphoma cell lines or ten thousand colon/lung cancer cells were seed in a volume of 80 μl in complete growth medium on 96-well plate. GSK3β3 inhibitors were added at the indicated concentration in a total volume of 100 μl. After treatment for 3 days, Cell Titer Glo 2.0 assays (Promega) were performed according to the manufacturer's instructions. Relative viability was calculated by measuring the luminescence value and normalizing it to baseline values of untreated cells. LY2090314 (S7063), CHIR99021 (S2924), CHIR98014 (S2745), 6-bromoindirubin-3-oxime (S7198) and Tideglusib (S2823) were bought from Selleck Chemicals and 9-ING-41 (AOB33534) was bought from AOBious.

### CRISPR-Mediated Gene Deletion

For non-viral gene deletion in human cells or mouse cells, Alt-R CRISPR-Cas9 guide RNAs and non-targeting control guide RNAs were purchased from IDT (Table 4). Chemically synthesized crRNAs (100 μmol 1⁻¹) and tracrRNAs (100 μmol 1⁻¹) were annealed by incubation at 95 C° for 5 min. Recombinantly produced Cas9 (40 μmol 1⁻¹) were then added to RNA mixture to produce RNA ribonucleoprotein (RNP) complexes. Electroporation was performed by using Neon™ Transfection system (Invitrogen). For experiments involving Ikzf1 and/or Ikzf3 deletion in mouse B-ALL cells and CTNNB1 deletion in BV173 cells, single cell derived colonies were generated to obtain fully knock-out cell lines.

Ctnnb1 deletion was introduced into mouse B-ALL cells by retroviral delivery vectors. Briefly, B-ALL cells were transduced with FUCas9mCherry vector (Table 5) and subsequently transduced with H1-gRNA-TetR-TagBFP vector (Table 5) carrying gRNA against Ctnnb1 or non-targeting control. Cells were sorted for BFP and mCherry expression. Expression of guide RNA was induced by addition of 1 g ml⁻¹ of Doxycycline. Single cell derived colonies were generated from these cells and used for further experiments.

### Colony Formation Assay

For colony forming assays, 10,000 mouse BCR-ABL1 or NRAS^{G12D} were grown on MethoCult medium (M3231 or M3630 (with IL-7) respectively, StemCell Technologies) in 3-cm diameter dishes with an extra dish filled with water to prevent evaporation. Colony forming assays with human leukemia/lymphoma cell lines were performed by plating 10,000 cells on MethoCult medium without human cytokines (H4230). For mouse pre-B cells 50.000 cells were grown on MethoCult medium with IL-7 (M3630). For colony forming assays assessing mouse myeloid progenitors, 10,000 cells were grown on MethoCult medium with mouse SCF, IL-3, IL-6, EPO (M3434). For human cord-blood derived colonies, 1000 CD34⁺ HSCs were plated on MethoCult medium with recombinant human SCF, IL-3, IL-6, EPO, G-CSF, GM-CSF (H4435). For all colony forming experiments, colonies were imaged and counted using GelCount (Oxford Optronix) and 7-14 days after plating.

### Gene Editing and Functional Analysis of Human HSCs

CD34⁺ cord blood hematopoietic stem cells (HSCs) were bought from All Cells and used in accordance with the guidelines approved by the Institutional Review Board of Yale University. CD34⁺ HSCs were cultured in StemSpan™ SFEM II medium with 1% P/S, 50 ng ml⁻¹ TPO (PeproTech), 50 ng ml⁻¹ SCF (PeproTech), and 100 ng ml⁻¹ Angpt15 (Miltenyi Biotec) for 2 days before electroporating with guides targeting CTNNB11 or control guide. 6 hours after electroporation, 100,000 cells were injected via the tail vein into unconditioned NSGW41 mice (6-8 weeks). 10-15 weeks after transplantation, peripheral blood was collected via submandibular vein and erythrocyte lysis was performed. At 15 weeks, mice were sacrificed and bone marrow, spleen and thymus were harvested and analyzed by flow cytometry.

### In Vivo Analysis of Leukemic Cells

Patient derived BCR-ABL1 ALL were labeled with firefly luciferase and selected by 25 μg ml⁻¹ blasticidin. 1 million PDX cells pre-treated with LY2090314 (10 μg ml⁻¹) or vehicle control for four hours. Cells were washed twice and then injected via the tail vein into sublethally irradiated (190 cGy) NSG mice (8-12 week-old, female). LY2090314 or vehicle control (dissolved in 5% DMSO, 45% PEG300 and PBS) were administered intraperitoneally at dose of 12.5 mg kg⁻¹ body weight twice every day for 20 times. The in vivo expansion and leukemia burden were monitored by luciferase bioimaging (Lago X; Accela). Briefly, D-luciferin (Promega) was dissolved in PBS and injected intraperitoneally at a dose of 2.5 mg per mouse 15 min before measuring luminescent. All mice were anesthetized by 5% isoflurane and continued during detection of light emission with 2% isoflurane introduced through a nose cone. When the mice get the signs of leukemia (hunched back, weight loss and inability to move), they were euthanized. Bone marrow and spleen were collected and flow cytometry analysis was performed to check leukemia engraftment.

### TMA Analysis

Patient biopsies were obtained in compliance with the internal review board of Yale University. Tissue microarrays (TMAs) were constructed with tumor types and normal controls. Formalin-fixed paraffin-embedded TMAs were cut at 4 microns. TMAs were processed on Ventana Discovery Ultra IHC automated stainer (Ventana Medical Systems, Roche Diagnostics, Indianapolis, USA). This includes deparaffinization, rehydration, endogenous peroxidase activity inhibition and antigen retrieval. The TMAs were stained with anti-human β-catenin monoclonal antibody (Clone #14, Ventana), followed by anti-Mouse HQ secondary antibody (DISCOVERY) and anti-HQ-HRP detection system (DISCOVERY). The stains were visualized with ChromoMap DAB Kit (DISCOVERY), counterstained with hematoxylin (Ventana) and coverslipped.

mRNA Sequencing and Data Analysis

RNA was isolated using Macherey-Nagel RNA extraction kit according to manufacturer's instructions. RNA concentration was measured by NanoDrop 1000 (Thermo Fisher Scientific) and RNA integrity was determined using Bioanalyzer (Agilent). Library construction of 280 ng total RNA for each sample was made using KAPA Stranded mRNA-Seq Kit (Illumina Platforms; Kapa Biosystems) using 10 cycles of PCR amplification. Libraries were purified using AxyPrep Mag PCR Clean-up kit (Axygen™). Each library was quantified using a Qubit fluorometer (Life Technologies) and the size distribution assessed using the 2100 Bioanalyzer (Agilent Technologies, Santa Clara, USA). Sequencing was performed on an Illumina® Hiseq 2500 (Illumina, San Diego, CA, USA) instrument using the TruSeq PE Cluster Kit V4-cBot-HS (Illumina®) to generate 101 bp Paired-end reads sequencing with v4 chemistry. Quality control of RNA-Seq reads was performed using FastQC v0.11.9, samtools v1.7 and Picard v2.23.8. Transcipts were quantified with Salmon v1.4.0¹, and reads aligned using STAR v2.7.6²to the mouse genome (mm10/GRCm38, gencode vM24). Downstream analysis was performed in R³—differential expression was analyzed with DESeq2 v1.30.1⁴ with standard models and normal shrinkage estimators. Gene set enrichment analyses were performed with fgsea v1.16.0 using log₂ fold change estimates from DESeq2 and gene sets from MSigDB or internal data as indicated. RNA-seq data was deposited to GEO with the accession number GSE196767.

### Co-Immunoprecipitation

Co-immunoprecipitation experiments were performed using Pierce Crosslink Magnetic IP/Co-IP kit according to manufacturer's instructions (Thermo Scientific). Anti-β-catenin antibody (14/β-catenin, BD Biosciences) or isotype control (107.3, BD Biosciences) were coupled to protein A/G magnetic beads and covalently cross-linked with 20 μmol 1⁻¹ disuccinimidyl substrate (DSS). The antibody cross-linked beads were incubated with cell lysate over night at 4 C°. For all Co-IP experiments, beads washed twice with IP/lysis wash buffer to remove non-bound material and eluted in a low-pH elution buffer that dissociates bound antigen from the antibody cross-linked beads. The enriched antigen in low-pH was immediately neutralized and subjected to Western blotting or Mass Spectrometry.

### Mass Spectrometry

Peptides were analyzed by LC-MS/MS using a Dionex UltiMate 3000 Rapid Separation LC (RSLC) systems and a Orbitrap mass spectrometer (ThermoFisher Scientific). 6 μl peptide samples were loaded onto the trap column, which was 150 μm×3 cm in-house packed with 3 μm C18 beads. The analytical column was a 75 μm×10.5 cm PicoChip column packed with 3 μm C18 beads (New Objectives). The flow rate was kept at 300 nl min⁻¹. Solvent A was 0.1% FA in water and Solvent B was 0.1% FA in ACN. The peptide was separated on a 120-min analytical gradient from 5% ACN/0.1% FA to 40% ACN/0.1% FA. The mass spectrometer was operated in data-dependent mode. The source voltage was 2.40 kV and the capillary temperature was 275° C. MS¹ scans were acquired from 400-2000 m/z at 60,000 resolving power and automatic gain control (AGC) set to 1×10⁶. The fifteen most abundant precursor ions in each MS¹ scan were selected for fragmentation. Precursors were selected with an isolation width of 1 Da and fragmented by collision-induced dissociation (CID) at 35% normalized collision energy in the ion trap. Previously selected ions were dynamically excluded from re-selection for 60 seconds. The MS² AGC was set to 3×10⁵. Proteins were identified from the MS raw files using Mascot search engine (Matrix science). MS/MS spectra were searched against the SwissProt human database. All searches included carbamidomethyl cysteine as a fixed modification and oxidized Met, deamidated Asn and Gln, acetylated N-term as variable modifications. Three missed tryptic cleavages were allowed. The MS¹ precursor mass tolerance was set to 10 ppm and the MS² tolerance was set to 0.6 Da. %10 false discovery rate cutoff was applied at the peptide level.

### Data Analysis for Proteomics Data

Downstream analysis of proteomic data sets was performed in R³; protein values were quantile normalized, and mixed imputation used to estimate missing values. Missing values were classified as missing not at random (MNAR) if proteins were detected for less than 2 replicates from a condition, and missing at random (MAR) otherwise. MNAR values were imputed by minimum probability, while MAR values were estimated by maximum-likelihood imputation using the MSnbase and DEP packages⁶. Normalized, imputed values were used in linear modelling and empirical Bayes testing for differentially enriched proteins between conditions using limma and DEP packages^{6,7}; differential enrichment results are given in pull-down experiments respectively. For visualization, fold change over Ig-background binding was further divided by average background binding detected in CRAPome to down-weight common contaminants⁸.

### ChIP Sequencing and Analysis

ChIP-Rx method was applied to mapping histone marks among various cell conditions⁹. In each experiment, B-ALL cells were counted and fixed with 1% formaldehyde for 10 min at room temperature and quenched by 125 mmol 1⁻¹ glycine. B-ALL cells were spiked with fixed Drosophila S2 cells, and subjected to nuclear extraction. Nuclei were lysed with SDS lysis buffer and sonicated by Bioruptor (Diagenode). After sonication, cell debris was removed by centrifugation (13,000 rpm, 10 min), while supernatant was diluted and pre-cleared by incubation with a blend of isotype control IgG, Dynabeads Protein A and Dynabeads Protein G (Invitrogen). ChIP was performed using antibodies specifically recognizing either H3K27ac (Active motif, #39133) or H3K4me3 (Millipore, MC315). ChIPed DNA was reverse-crosslinked overnight at 65° C., and purified by QIAquick PCR purification kit (Qiagen). ChIP-seq libraries were constructed by a SMARTer ThruPLEX DNA-seq Kit (Takara) and subjected to Illumina deep sequencing. For ChIP-seq of IKZF1 and IKZF3, B-ALL cells were crosslinked by 2 mmol 1⁻¹ disuccinimidyl glutarate for 45 min and 1% formaldehyde for 10 min at room temperature before chromatin enrichment and library construction. These antibodies were used for ChIP-seq of IKZF1 (GeneTex, GTX129438) and IKZF3 (CST, D1C1E). β-catenin ChIP was performed by Active Motif, Inc. (Carlsbad, CA, USA) using the monoclonal antibody CAT-15 (Thermo). Quality control was performed using FastQC v0.11.9, and ChIPQC. Reads were aligned with BWA v0.7.17¹⁰ against the mouse genome (mm10/GRCm38, gencode vM24). Peak calling was performed with MACS2 v2.2.7.1. Downstream analysis was performed in R³—differential binding was analysed with DiffBind v3.0.15¹¹ peaks with -log 10 q-value >10 in 2 or more conditions were retained after black and grey-listing. For transcription factors (IKZF1, IKZF3 and CTNNB1) within-peak normalization was applied, while whole-genome normalization was applied to histone modification data. Annotation was performed with ChIPpeakAnno” to the closest TSS excepting the BENC enhancer region which was manually annotated as described in Bahr et al. 2018¹³. CHIP-seq data was deposited to GEO with accession number GSE196745.

### Quantification and Statistical Analysis

Data are shown as mean±s.d. unless stated. Statistical analysis was performed by GraphPad Prism 7 (GraphPad Software Inc.) using unpaired two-tailed t test or log-rank test as indicated in figure legends. Significance was considered at P<0.05. Kaplan-Meier survival analysis was used to estimate OS with GraphPad Prism 7. The investigators were not blinded to allocation during experiments and outcome assessment. Experiments were repeated to ensure reproducibility of the observations. IC50 values were estimated using default dose-response models in dre v3.0.1¹⁴ with an upper limit of 1.

### Data Availability

The mutation data for CTNNB1, APC, AXIN1, AXIN2 and GSK3β genes were acquired from https://cancer.sanger.ac.uk/cosmic. The data for RIC staining of β-catenin was obtained from https://www.proteinatlas.org/. The RNA-seq data and mass spectrometry data for β-catenin and IKZF1 in human cancer cell lines were obtained from depmap.org/portal/download/. Data for drug-response of solid tumor cell lines to were acquired from depmap.org/portal/download/. Hallmark Myc-Targets V1 and Hallmark Myc-Targets V2 gene sets were acquired from MSigDB. RNA-seq data was deposited to GEO with the accession number GSE196767 and ChIP-seq data was deposited to GEO with accession number GSE196745. All other data are available from the corresponding author upon reasonable request.

### REFERENCES

1. Patro R., et al. Salmon provides fast and bias-aware quantification of transcript expression. Nature Method 14, 417-419 (2017).
2. Dobin, C. A. et al. STAR: ultrafast universal RNA-seq aligner. Bioinformatics 29, 15-21 (2013).
3. R Core Team. R: A language and environment for statistical computing. R Foundation for Statistical Computing, Vienna, Austria. URL https://www.R-project.org/(2021).
4. Love, M. I., Huber, W., Anders, S. Moderated estimation of fold change and dispersion for RNA-seq data with DESeq2. Genome Biology 15, 550 (2014).
5. Zhang X. et al. Proteome-wide identification of ubiquitin interactions. Nat Protoc 13, 530-550 (2018).
6. Gatto L., Lilley K. S. MSnbase-an R/Bioconductor package for isobaric tagged mass spectrometry data visualization, processing and quantitation. Bioinformatics 28, 288-289 (2012).
7. Ritchie M. E. et al. limma powers differential expression analyses for RNA-sequencing and microarray studies. Nucleic Acids Research 43, e47 (2015).
8. D Mellacheruvu et al. The CRAPome: a Contaminant Repository for Affinity Purification Mass Spectrometry Data. Nature Method 10, 730-736 (2013).
9. Orlando D. A. et al. Quantitative ChIP-Seq normalization reveals global modulation of the epigenome. Cell Rep. 9, 1163-70 (2014).
10. Li, Heng, and Richard Durbin. Fast and accurate short read alignment with Burrows-Wheeler transform. Bioinformatics 25, 1754-1760 (2009).
11. Ross-Innes, C. S. et al. Differential oestrogen receptor binding is associated with clinical outcome in breast cancer. Nature 481, 389-393 (2012).
12. Zhu J. L. et al. ChIPpeakAnno: a Bioconductor package to annotate ChIP-seq and ChIP-chip data. BMC Bioinformatics 11, 237 (2010).
13. Bahr C. et al. A Myc enhancer cluster regulates normal and leukaemic haematopoietic stem cell hierarchies. Nature 553, 515-520 (2018).
14. Ritz C., Baty F., Streibig J. C., Gerhard D. Dose-Response Analysis Using R. Plos One 10, e0146021 (2015).

### Example 1. Lack of β-Catenin Signaling in B-Cell Development and B-Lymphoid Malignancies

The WNT/β-catenin pathway is involved in fundamental processes including embryonic development, organogenesis, and tissue homeostasis¹⁻⁶. β-catenin protein levels are tightly regulated by β-catenin-degradation, which is initiated by Glycogen Synthase Kinase 3β (GSK3β) and the scaffolding proteins Axin1, Axin2 and Adenomatous Polyposis Coli (APC)⁷⁻⁹. In the absence of Wnt ligands, β-catenin is phosphorylated by GSK3β on N-terminal serine and threonine residues encoded by exon 3 for subsequent proteasomal degradation⁸⁻⁹. Conversely, Wnt ligands stabilize β-catenin and induce its nuclear accumulation to promote transcription of Wnt target genes including MYC^{4-6,9}. β-catenin functions as a central driver of MYC-expression, proliferation, and survival in multiple epithelial, neuronal, and mesenchymal lineages⁴⁻⁶, but is dispensable for hematopoietic development¹⁰⁻¹². Intermediate levels of β-catenin signaling were shown to promote survival and proliferation of hematopoietic stem and progenitor cells¹³, as well as multiple stages of T-cell development¹⁴⁻¹⁶ Targeted removal of GSK3β-phosphorylation sites and β-catenin accumulation in Ctnnb1^{ex3fl/+} mice, had detrimental effects on hematopoiesis¹⁷⁻¹⁸, and suppressed early T-cell development¹⁹. This contrasts with studies in colon cancer, melanoma, and other epithelial cancers, where genetic accumulation of β-catenin results in acceleration of proliferation and malignant transformation^{2,20-21}. The role of β-catenin signaling in myeloid and T-cell malignancies is controversial: While earlier studies demonstrated an important role of β-catenin in the initiation of myeloid (AML, CML)²²⁻²⁴ and T-cell leukemia (T-ALL)²⁵⁻²⁶, a new genetic mouse model provided evidence that T-cell development and AML leukemia-initiation do not require β-catenin¹².

β-catenin lacks a DNA binding domain and interacts with TCF7-family transcription factors²⁻³ in epithelial, mesenchymal, neuronal, and myeloid cells to induce transcriptional activation of WNT-target genes including MYC⁴⁻⁶. Here it was shown that genetic and pharmacological β-catenin accumulation selectively impact B-lymphoid cells by transcriptional repression of MYC. This unexpected outcome of β-catenin signaling in B-lymphocytes was predicated on previously unrecognized repressive complexes between β-catenin and lymphoid-specific Ikaros (IKZF1 and IKZF3) transcription factors.

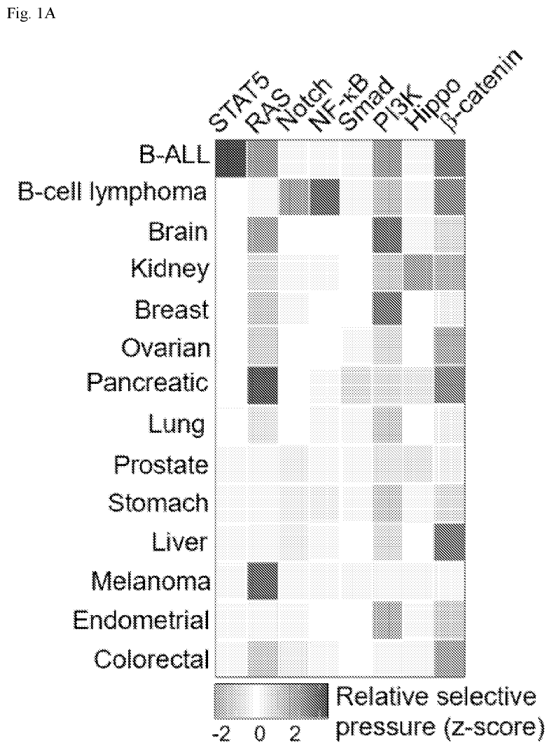

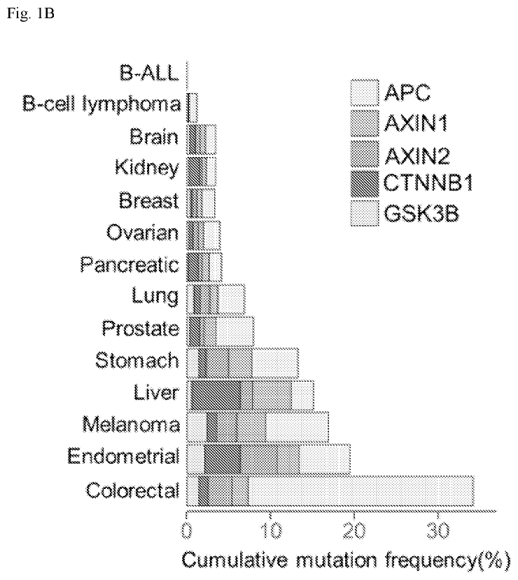

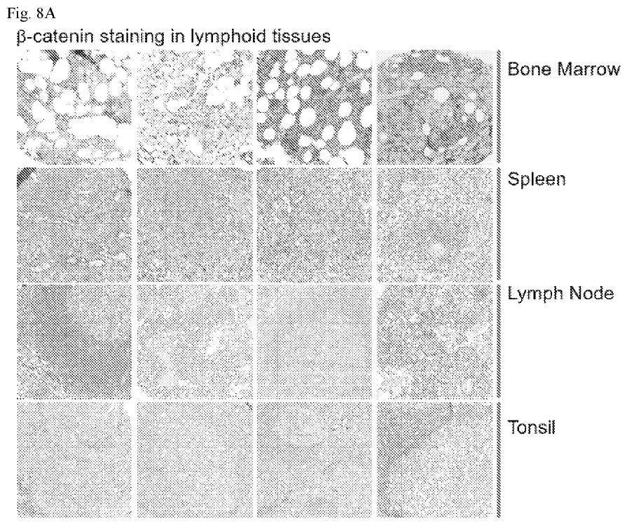

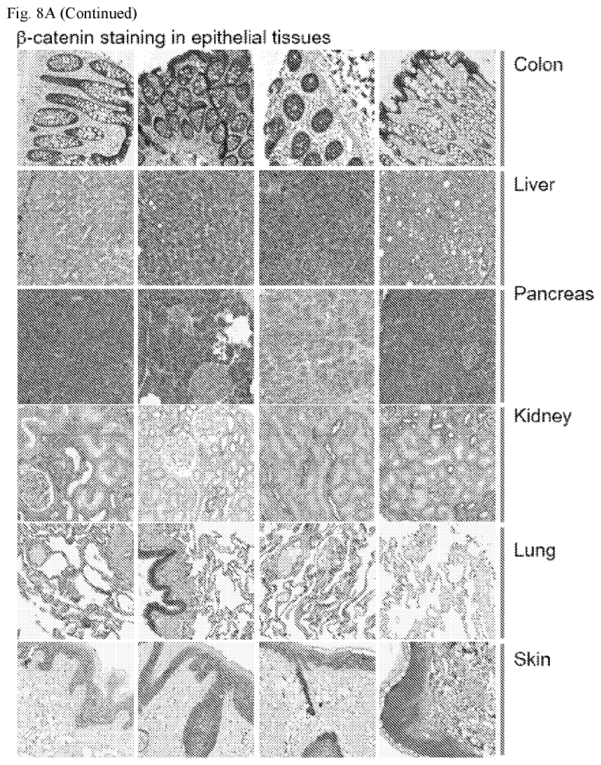

In a computational pan-cancer analysis of oncogenic drivers in eight defined signaling pathways, activating lesions of the β-catenin signaling pathway were strongly selected in 12 cancer types but showed evidence of negative selective pressure in B-cell acute lymphoblastic leukemia (B-ALL) and B-cell lymphoma (FIG. 1A). Studying individual β-catenin pathway lesions (FATHMM score >0.5) in 66,949 cancer samples encompassing 16 types of solid tumors and hematological malignancies²⁷, frequent mutations were found, including of CTNNB1 itself (1.6%) and its negative regulators APC (8.9%), AXIN1 (1.4%), AXIN2 (1.1%) and GSK3B (0.6%). In contrast, among 2,137 B-cell malignancies, cases with activating β-catenin pathway lesions were markedly underrepresented (expected 264, observed 17, x test P=1.2 E-12; FIG. 1B, Table 8). Since mutation frequencies for two of the five oncogenic drivers of β-catenin signaling were also reduced in T-cell malignancies (AXIN2, GSK3β; Table 1), it was examined whether these differences reflect a general reduction of β-catenin signaling in lymphoid lineages, encompassing B-, T- and NK-cells. To this end, β-catenin signaling was measured in Axin2-turquoise reporter transgenic mice²⁸. While β-catenin signaling was clearly detectable in T-cells and NK-cells, this was not the case for B-cells (FIG. 1C), suggesting that attenuation of β-catenin signaling represents a unique feature of B-lymphocytes. Immunohistochemistry confirmed low baseline levels of β-catenin protein expression in B-lymphoid tissues (n=24) compared to consistently high β-catenin expression in myeloid bone marrow cells, as well as epithelial and mesenchymal cell types (n=30; FIGS. 1F, 8A). Detailed flow cytometry analysis of Axin2-turquoise transgenic mice corroborated that, unlike thymic and mature T-cell development, B-cells consistently lacked β-catenin signaling throughout early and late stages of development (FIGS. 8B-8C). Interestingly, β-catenin mRNA levels were comparable between B-cell malignancies and solid tumors (RNA-seq; FIG. 1D). While β-catenin mRNA levels were similar in B-cells compared to other cell types, β-catenin protein levels (reverse phase protein arrays, proteomics) were markedly suppressed in B-lymphoid cell lines (FIG. 1D), suggesting that B-cells may be more adept than other cell types at clearing β-catenin protein by GSK3β-mediated degradation^{8,9}. Mirroring low β-catenin protein levels, B-cell leukemia and lymphoma cells were resistant to CRISPR-mediated deletion of CTNNB1²⁹, while solid tumors, in particular gastrointestinal tumors, were sensitive to loss of β-catenin (FIG. 1E). Low baseline β-catenin protein expression was confirmed in a panel of 84 B-cell malignancies compared to consistently high β-catenin protein levels in epithelial cancers (lung, colon, melanoma; n=27) by immunohistochemistry (FIG. 9). B-cell-specific lack of nuclear β-catenin was further corroborated by nuclear and cytoplasmic cell fractionation and Western blot analysis: Nuclear accumulation of β-catenin was consistently detected at high levels in epithelial tumor cell lines (n=16), including lung and colon cancer and malignant melanoma, but not in B-ALL (n=7) and mature B-cell malignancies (n=15; FIGS. 1G, 9).

TABLE 8
Genetic lesions affecting β-catenin stability and function

| | Gene | | | | | | | | | | | | | | |
|---|---|---|---|---|---|---|---|---|---|---|---|---|---|---|---|
| | CTNNB1 | | | APC | | | AXIN1 | | | AXIN2 | | | GSK3β | | |
| Tumor Type | Total | Mutated | Freq | Total | Mutated | Freq | Total | Mutated | Freq | Total | Mutated | Freq | Total | Mutated | Freq |
| B-cell | 2,137 | 2 | 0.09 | 2,00 [symbol] | 8 | 0.40 | 2, [symbol] 48 | 3 | 0.15 | 1,998 | 2 | 0.10 | 1,930 | 2 | 0.10 |
| T-cell | 839 | 8 | 0.72 | 859 | 19 | 2.21 | 614 | 2 | 0.33 | [symbol] 29 | 1 | 0.1 [symbol] | 830 | 0 | 0.00 |
| Myeloid | 1,234 | 3 | 0.24 | 1,328 | 1 [symbol] | 0.7 [symbol] | 1,220 | 14 | 1.16 | 1,210 | 14 | 1.1 [symbol] | 1,210 | 1 [symbol] | 0.83 |
| Breast | [symbol] ,911 | 16 | 0.23 | 8,503 | 130 | 1. [symbol] 3 | [symbol] ,789 | 44 | 0. [symbol] | 6,52 [symbol] | 33 | 0. [symbol] 1 | [symbol] ,45 [symbol] | 2 [symbol] | 0.39 |
| Brain | 5,623 | 38 | 0.68 | 4,204 | [symbol] 0 | 1.17 | 3,282 | 19 | 0. [symbol] 8 | 3,247 | 18 | 0.55 | 3,123 | 10 | 0.32 |
| Cervical | 580 | 4 | 0.69 | 51 [symbol] | 14 | 2.72 | 400 | 2 | 0. [symbol] 0 | 370 | 4 | 1.08 | 3 [symbol] | 1 | 0.27 |
| Endometrial | 2,544 | 122 | 4.80 | 1,261 | 76 | 6.03 | 995 | 2 [symbol] | 2.61 | 1,017 | 44 | 4.33 | [symbol] 37 | 20 | 2.13 |
| Kidney | 4,775 | 59 | 1.24 | 2,998 | 33 | 1.10 | 2,631 | 13 | 0.49 | 2, [symbol] 34 | [symbol] | 0.30 | 2, [symbol] 17 | 7 | 0.27 |
| Colorectal | 12,194 | 139 | 1.14 | 15,412 | 4044 | 26.24 | 4,224 | 75 | 1.78 | 4, [symbol] 17 | 119 | 2.58 | 4,048 | 52 | 1.28 |
| Liver | 7,178 | 4 [symbol] 8 | [symbol] .52 | 2,700 | 69 | 2.56 | 3,273 | 152 | 4. [symbol] 4 | 2,481 | 36 | 1.45 | 2,301 | 12 | 0.52 |
| Lung | 6,648 | 50 | 0.7 [symbol] | 6,258 | 197 | 3.15 | 4,357 | 42 | 0.96 | 4,415 | [symbol] 1 | 1.16 | 4,545 | 36 | 0.79 |
| Ovarian | 2,570 | 13 | 0.51 | 1, [symbol] 41 | 30 | 1.9 [symbol] | 1,248 | 8 | 0.64 | 1,248 | 7 | 0.56 | 1,215 | [symbol] | 0.25 |
| Pancreatic | 3,258 | 37 | 1.14 | 3,048 | 45 | 1.48 | 2,307 | 19 | 0.82 | 2,273 | 11 | 0.48 | 2,285 | 4 | 0.18 |
| Prostate | 4,324 | 50 | 1.16 | 4,224 | 192 | 4.55 | 3, [symbol] 13 | 55 | 1.44 | 3,764 | 22 | 0.58 | 3,225 | 11 | 0.34 |
| Stomach | 4,024 | 32 | 0.80 | 3,8 [symbol] 0 | 1 [symbol] 0 | 4.68 | 1,736 | 34 | 1.96 | 1,814 | 34 | 1.87 | 1,627 | 15 | 0.92 |
| Melanoma | 2,110 | 2 [symbol] | 1.23 | 1,000 | 75 | 7.50 | 461 | 16 | 3.47 | [symbol] 13 | 12 | 2.34 | 416 | 10 | 2.40 |
| B-cell | 2,137 | 2 | 0.09 | 2,0 [symbol] | 8 | 0.4 [symbol] | 2,048 | 3 | 0.15 | 1,998 | 2 | 0.10 | 1,930 | 2 | 0.10 |
| Other | [symbol] 4,812 | 10 [symbol] 3 | 1. [symbol] 4 | 57,701 | [symbol] 164 | 8.95 | 37,350 | [symbol] 21 | 1.39 | 3 [symbol] ,858 | 414 | 1.13 | 35,201 | 216 | 0.61 |
| Pan Cancer | 6 [symbol] ,949 | 1,0 [symbol] 5 | 1. [symbol] 9 | 59,707 | [symbol] ,172 | 8.66 | 39,398 | [symbol] 24 | 1.33 | 38, [symbol] 5 [symbol] | 41 [symbol] | 1.08 | 37,131 | 218 | 0.59 |

| | Mutation frequency | | | | | | | | | | | | | | |
|---|---|---|---|---|---|---|---|---|---|---|---|---|---|---|---|
| | Ex- | | | Ex- | | | Ex- | | | Ex- | | | Ex- | | |
| | pect | Observ | P= | pect | Observ | P= | pect | Observ | P= | pect | Observ | P= | pect | Observ | P= |
| B- | 34.07 | 2 | [symbol] .39E−08 | 171.72 | 8 | 1.3E−35 | 26.24 | [symbol] | 2E−0 [symbol] | 20.9 | 2 | 0.0003 | 11.1 | 2 | 0.0008 |
| cell | | | | | | | | | | | | | | | |

[symbol]

indicates data missing or illegible when filed

### Example 2. B-Lymphoid Cells Critically Depend on GSK3β-Mediated Degradation of β-Catenin Protein

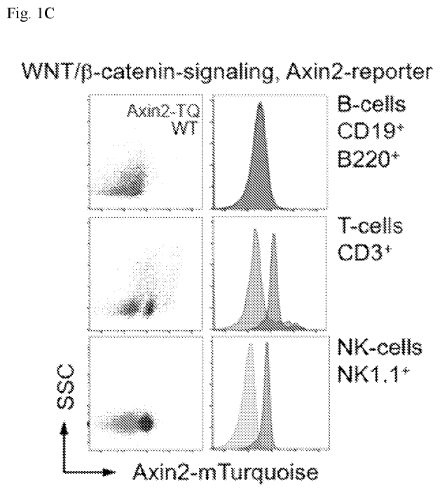

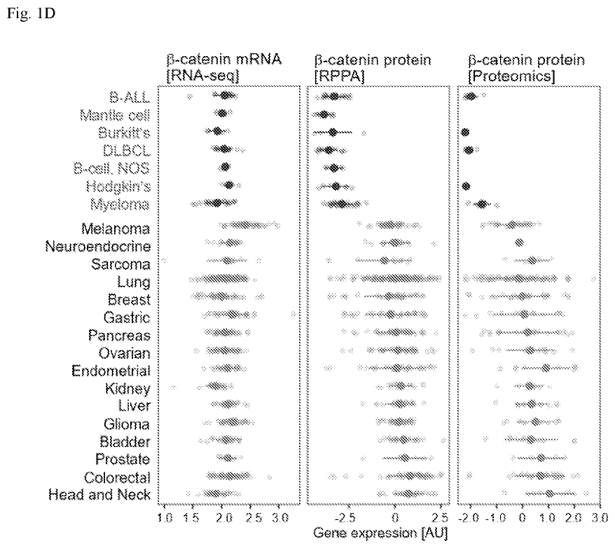

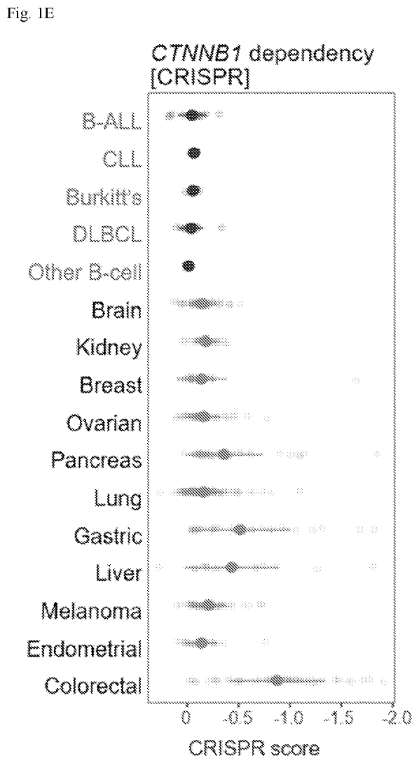

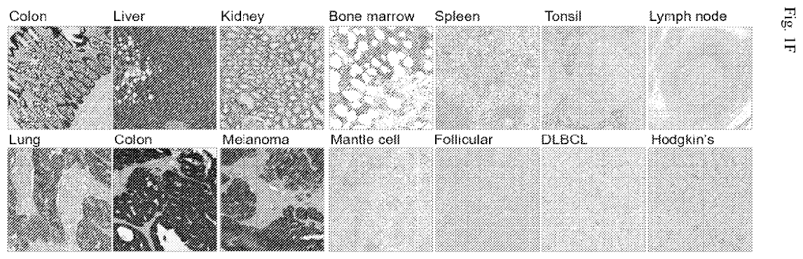

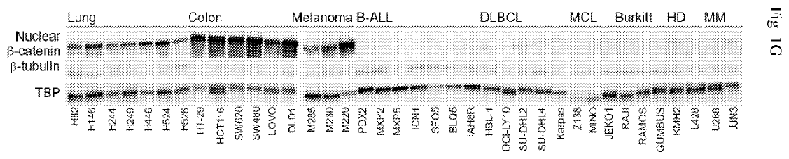

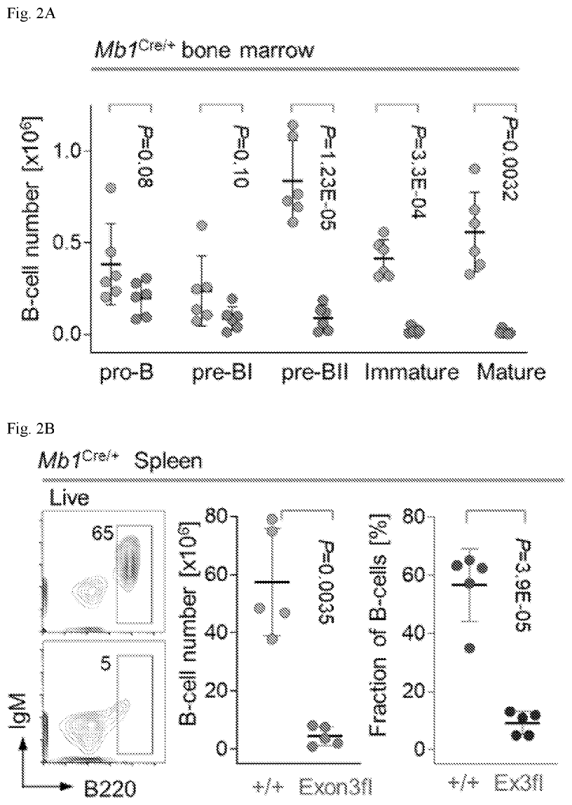

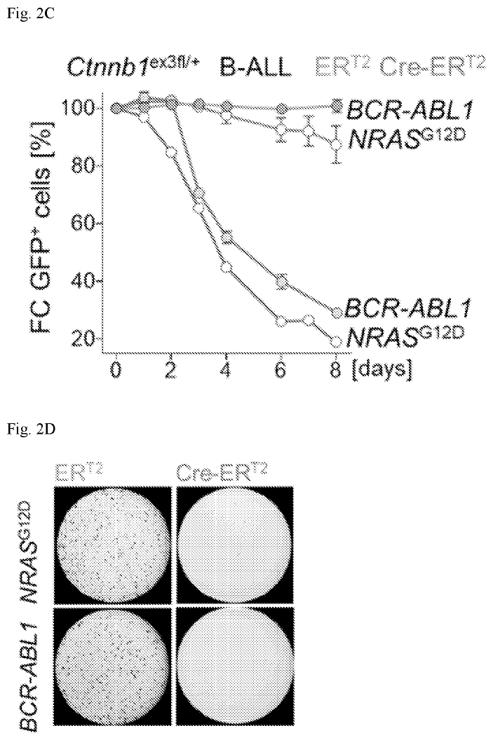

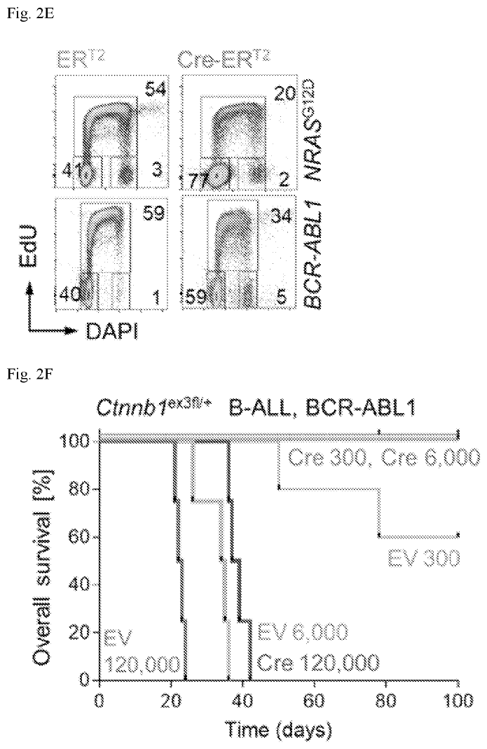

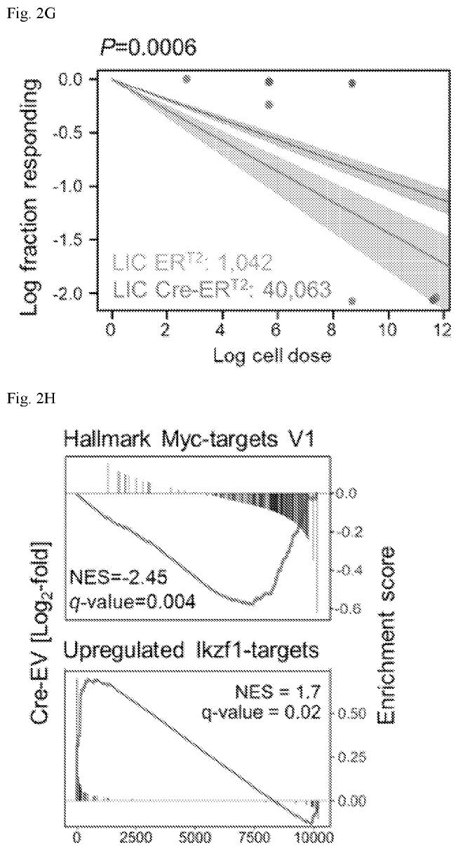

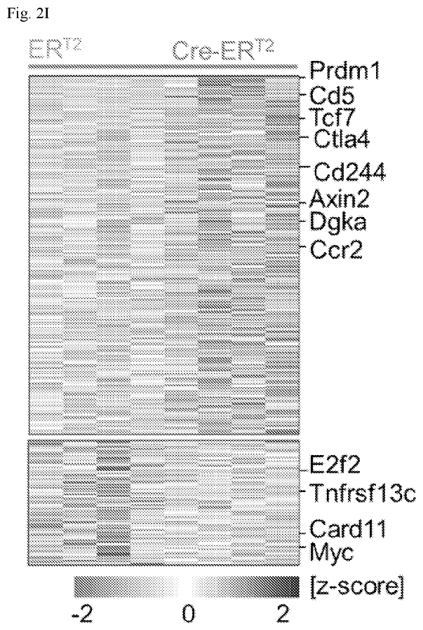

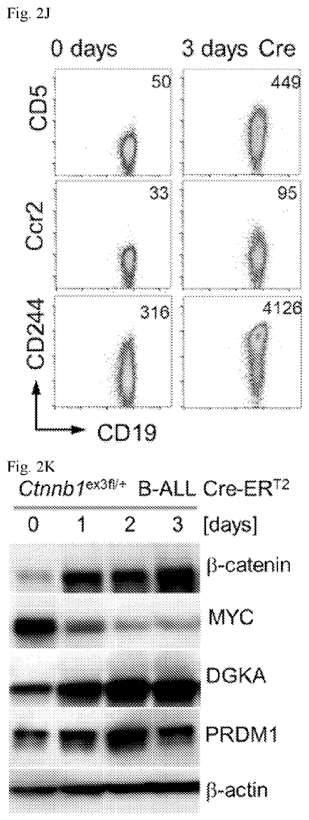

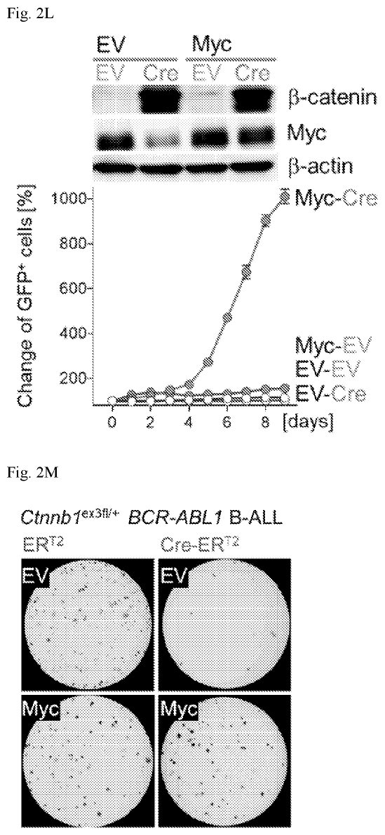

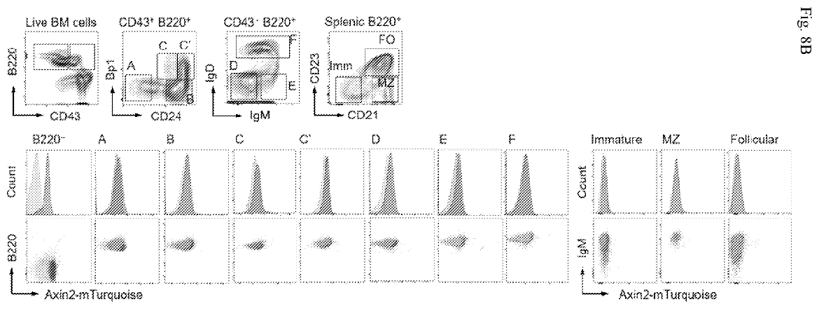

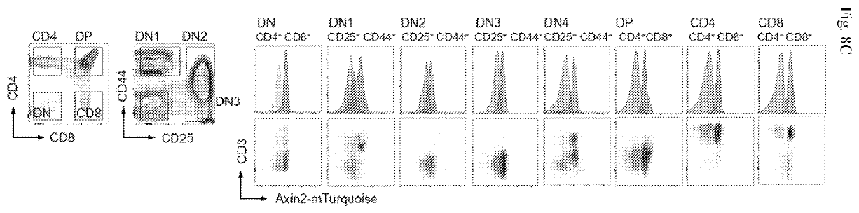

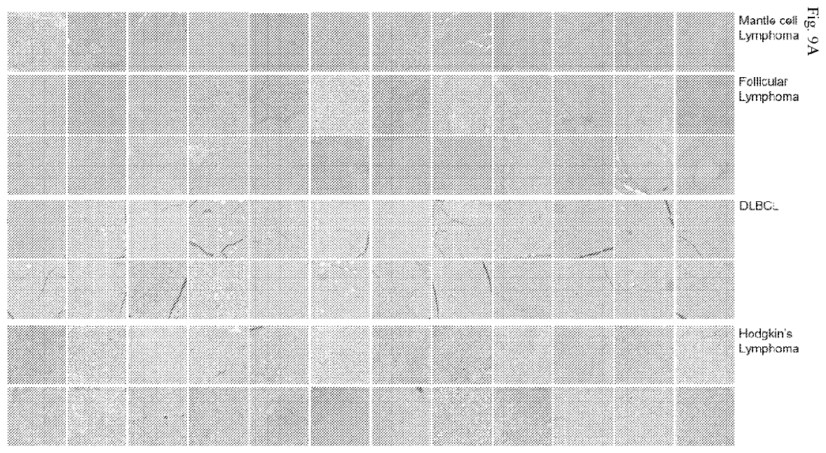

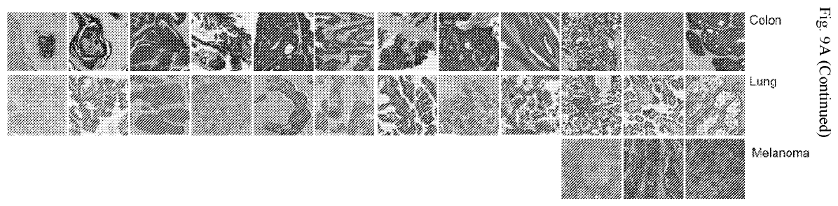

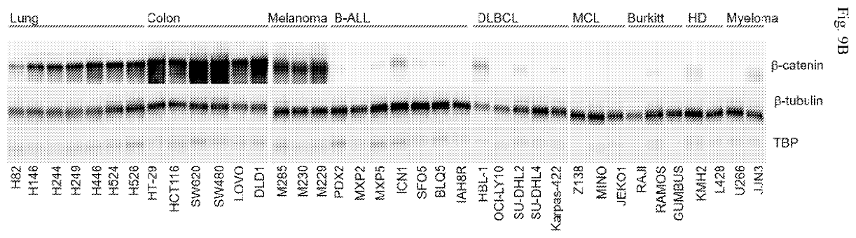

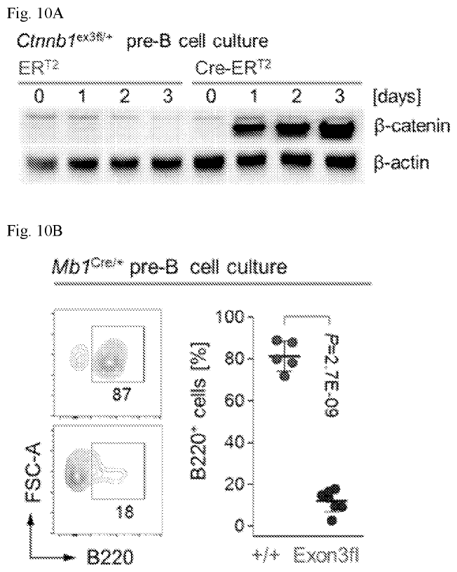

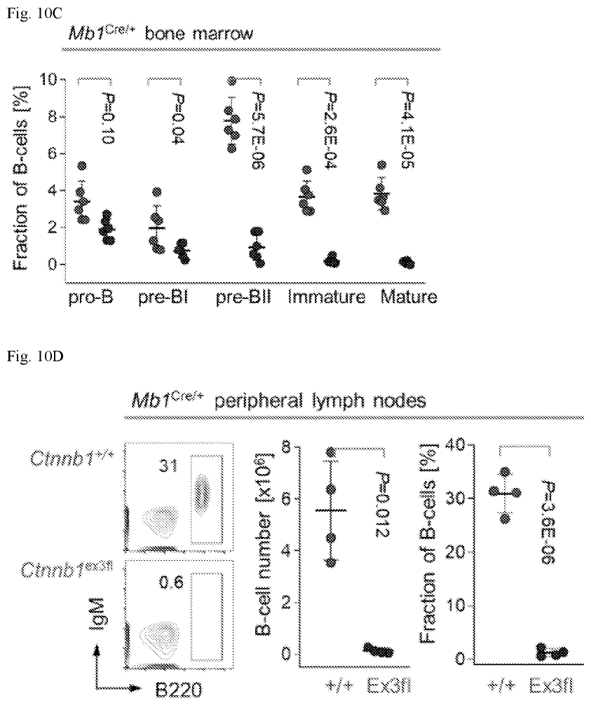

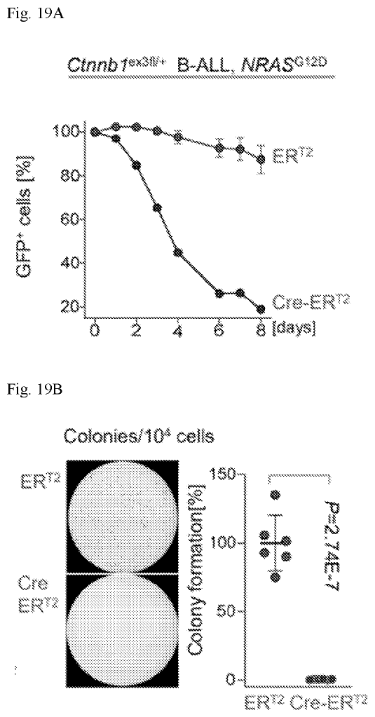

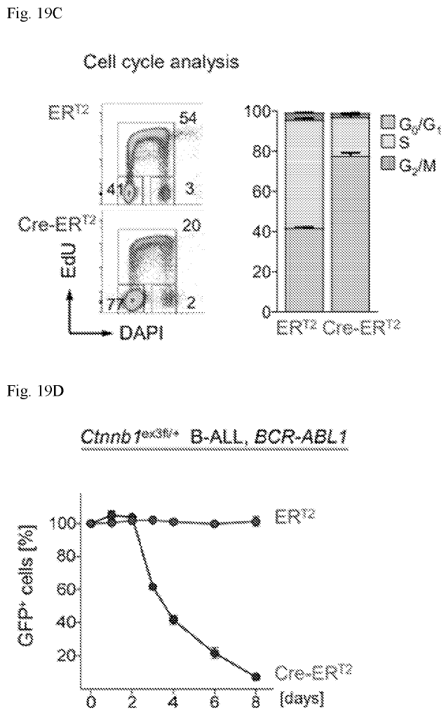

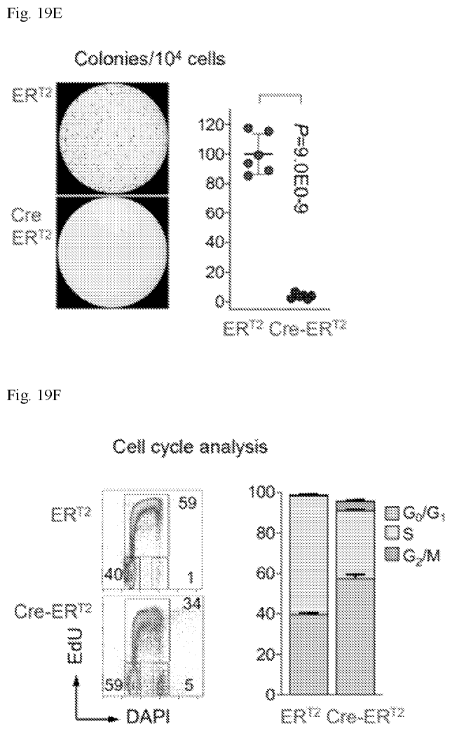

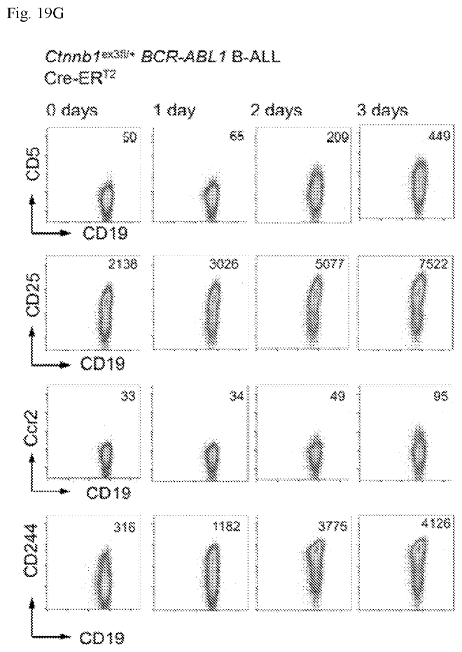

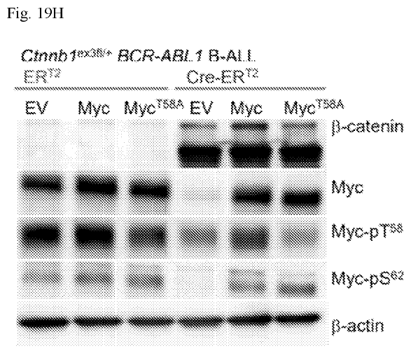

To follow up on the observation that B-cells consistently lacked expression and activity of β-catenin protein despite relatively high mRNA levels, the GSK3β-dependent protein degradation of β-catenin was studied in B-lymphoid cells. To this end, Ctnnb1^{ex3fl/+} mice³⁰ were crossed it a B-cell-specific Mb1-Cre deleter strain. Expression of Cre in this model recapitulated β-catenin accumulation as the common outcome of cancer-associated genetic lesions in this pathway, that are commonly found throughout the entire spectrum of cancer but not in B-lymphoid malignancies (FIG. 1B; Table 8). Cre-mediated removal of GSK3β-phosphorylation sites of β-catenin abrogated GSK3β-mediated degradation, resulting in β-catenin accumulation from earliest stages of B-cell development. While pro-B and pre-BI cells (Hardy fractions A-C) tolerated β-catenin accumulation, B-lymphopoiesis beyond pre-BCR⁺ stages (Hardy fractions C′-F and mature B-cells) of development was profoundly suppressed in vivo (FIGS. 2A-2B, 10). To model defective GSK3β-mediated degradation of β-catenin in common subtypes of B-ALL, B-cell precursors from Ctnnb1^{ex3fl/+} mice were transduced with BCR-ABL1 and NRAS^{G12D} oncogenes and tamoxifen-inducible Cre (Cre-ER^{T2}) or empty vector control (ER^{T2}). Inducible β-catenin accumulation subverted competitive fitness of B-ALL cells, abolished colony formation and induced G₀/G₁ phase cell cycle arrest (FIGS. 2C-2E). Transplant experiments revealed that β-catenin stabilization in BCR-ABL1-driven B-ALL cells subverted leukemia-initiation in vivo: Compared to controls, β-catenin stabilization reduced the frequency of leukemia-initiating cells by about 40-fold (FIGS. 2F-2G).To model β-catenin accumulation in common subtypes of B-ALL, B-cell precursors from Ctnnb1^{ex3fl/+} were transduced with BCR-ABL1 and NRAS^{G12D} oncogenes and tamoxifen-inducible Cre (Cre-ER^{T2}) or empty vector control (ER^{T2}). Genetic stabilization of β-catenin subverted competitive fitness of B-ALL cells, abolished colony formation and induced G₀/G₁ phase cell cycle arrest (FIGS. 19A-19F).

### Example 3. β-Catenin-Accumulation in B-Lymphoid Cells Results in Transcriptional Repression of MYC

RNA-seq analysis of β-catenin-dependent gene expression changes revealed enrichment for two principal gene sets, namely suppression of Myc-targets and of activation of transcriptional targets of the Ikaros zinc finger protein IKZF1 (FIG. 2H). While Myc and E2f were repressed, molecules related to B-cell anergy (Prdm1, Cd5, Dgka, Cd244, Ctla4), and β-catenin signaling (Tcf7, Axin2) were strongly upregulated upon β-catenin-activation (FIGS. 2I-2K). β-catenin-mediated repression of Myc in murine B-ALL cells was in striking contrast to previous findings of MYC as a classical target of β-catenin-mediated transcriptional activation in epithelial cells⁴⁻⁶. Consistent with gene set enrichment analyses (FIG. 2H), genetic rescue experiments identified suppression of Myc as central mechanistic element of β-catenin-mediated cell death in mouse B-ALL cells: Reconstitution of MYC expression rescued the deleterious effects of β-catenin-accumulation and restored both colony formation and competitive fitness of B-ALL cells (FIGS. 2L-2M).

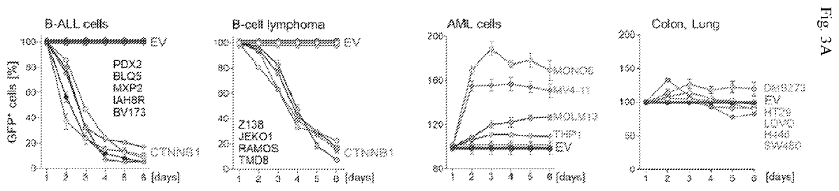

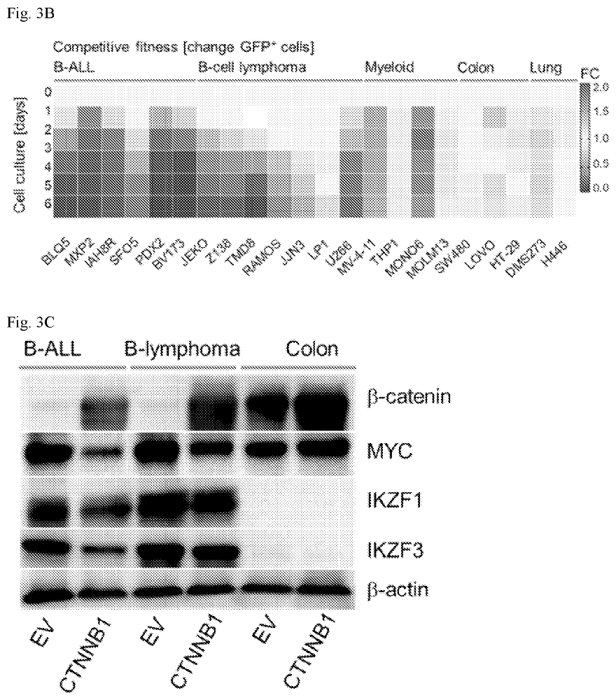

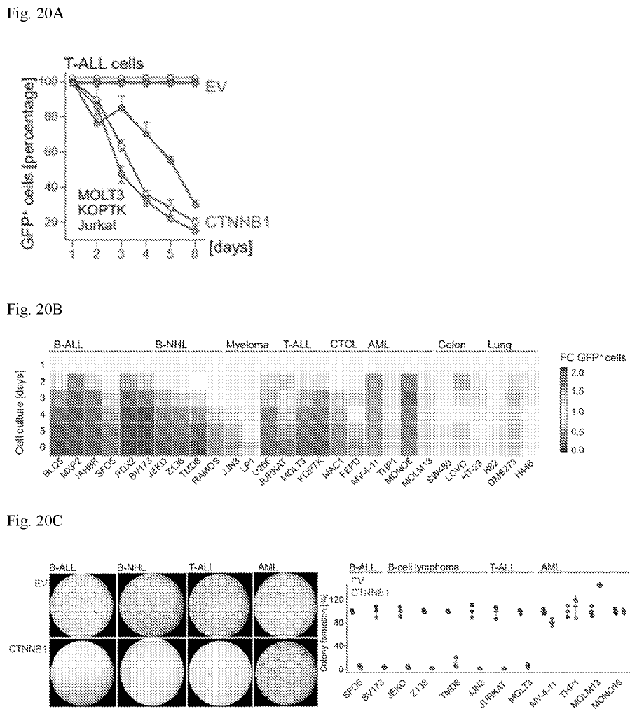

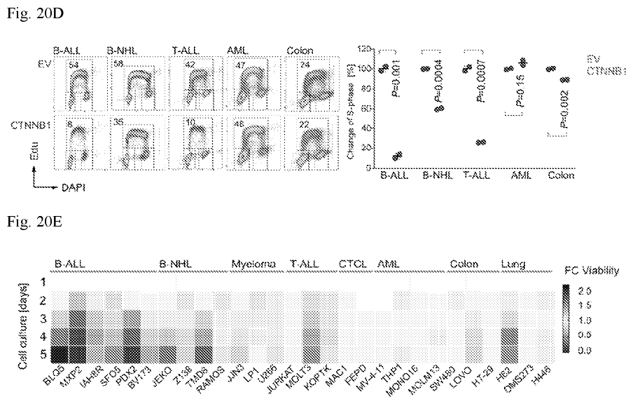

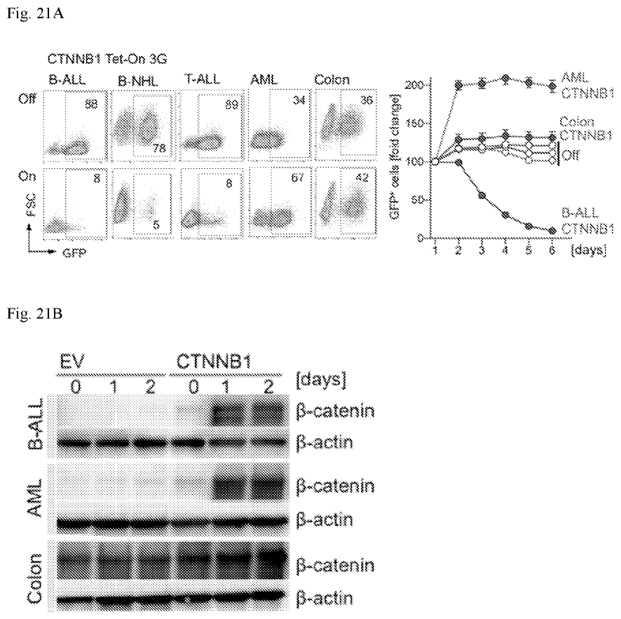

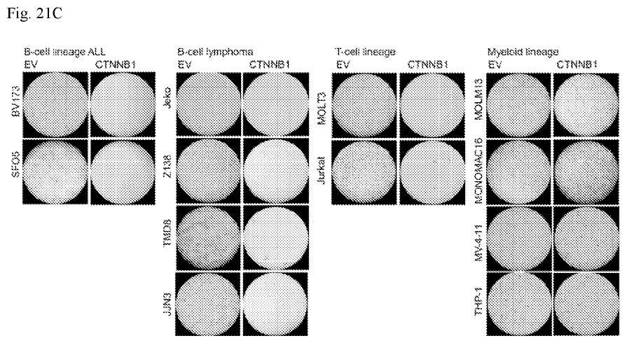

To determine functional consequences of β-catenin accumulation in human cells, doxycycline-inducible expression of stabilized β-catenin was studied in B-lymphoid (B-ALL, mantle cell, Burkitt's lymphoma, DLBCL; n=13) cell lines and patient-derived xenografts (PDX), myeloid leukemia (n=4), and colon and lung epithelial cell lines (n=6). In B-lymphoid cells, inducible β-catenin accumulation suppressed MYC-expression, compromised clonal fitness, colony formation, cell proliferation and induced cell death (FIGS. 3A-3F). Inducible β-catenin accumulation had no significant effects in colon and lung cells and increased competitive fitness and colony formation in myeloid leukemia cells (FIGS. 3A-3E). Together, these results suggest that B-lymphoid cells fundamentally differ from myeloid and epithelial cell types in that they are not permissive to accumulation of β-catenin. To determine functional consequences of β-catenin accumulation in human cells, doxycycline-inducible expression of stabilized β-catenin in 18 lymphoid (B-ALL, lymphoma, T-ALL, PTCL) cell lines and patient-derived xenografts (PDX), four myeloid leukemia, and six colon and lung epithelial cell lines was studied. In B- and T-lymphoid cells, inducible β-catenin accumulation compromised clonal fitness, colony formation, cell proliferation and induced cell death. In contrast, inducible β-catenin accumulation had no significant effects in colon and lung epithelial cells and increased competitive fitness and colony formation in myeloid leukemia cells (FIG. 20; FIG. 21). Together, these results suggest that lymphoid cells fundamentally differ from myeloid and epithelial cell types in that they are not permissive to activation of β-catenin.

### Example 4. β-Catenin Forms Repressive Complexes with Ikaros Factors and NuRD Components in B-Lymphoid Cells

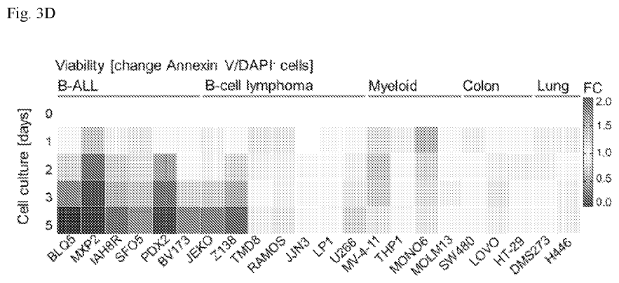

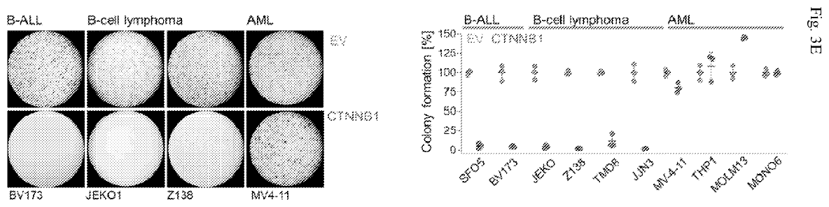

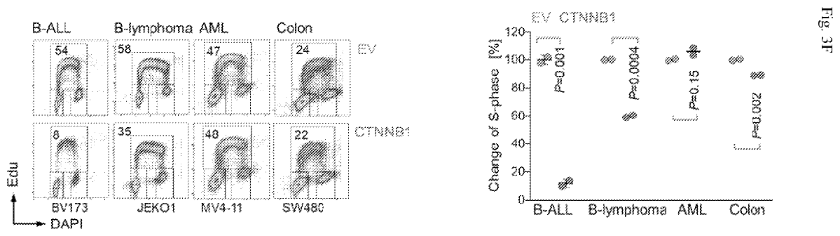

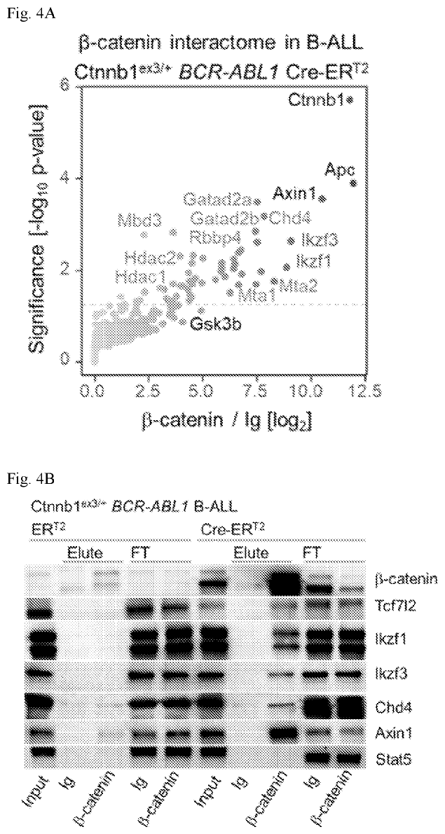

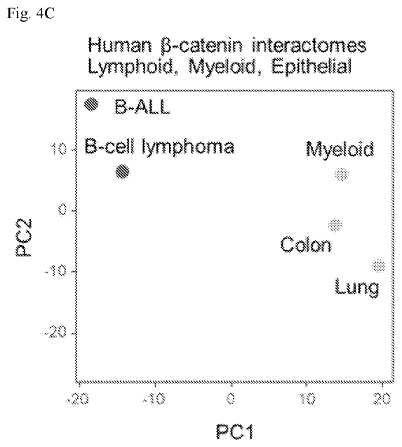

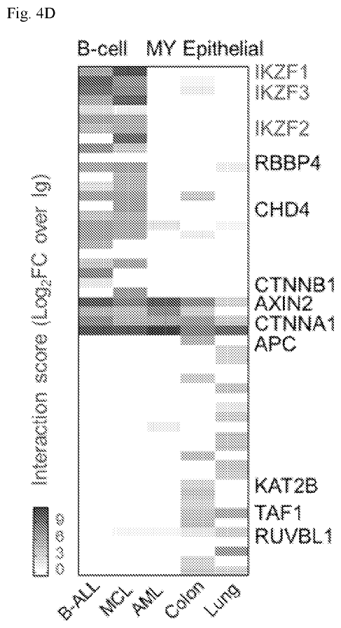

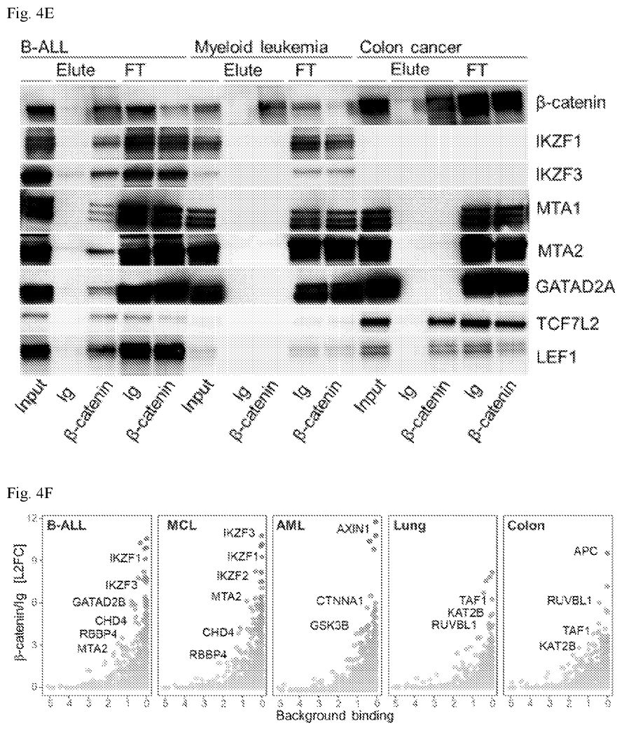

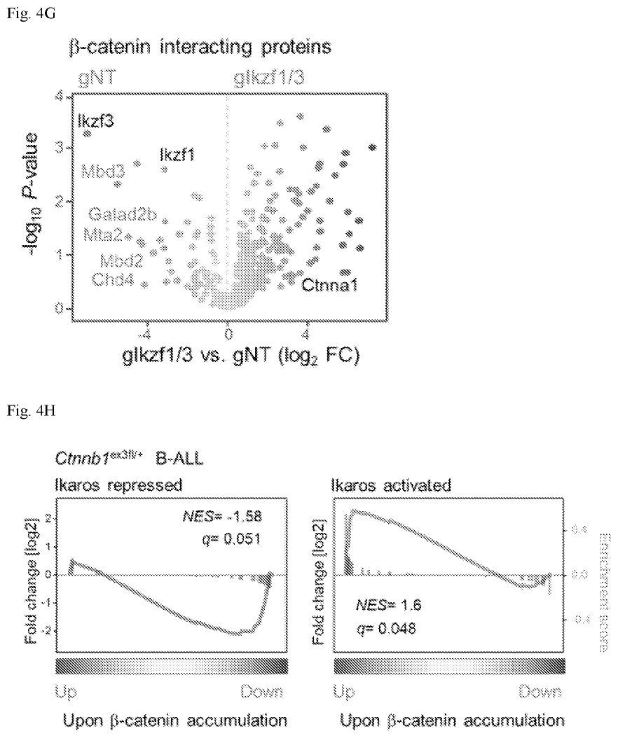

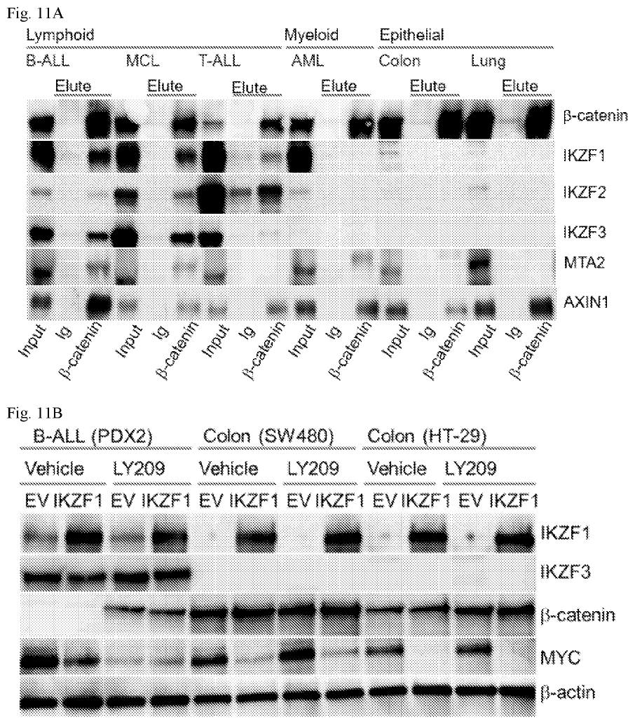

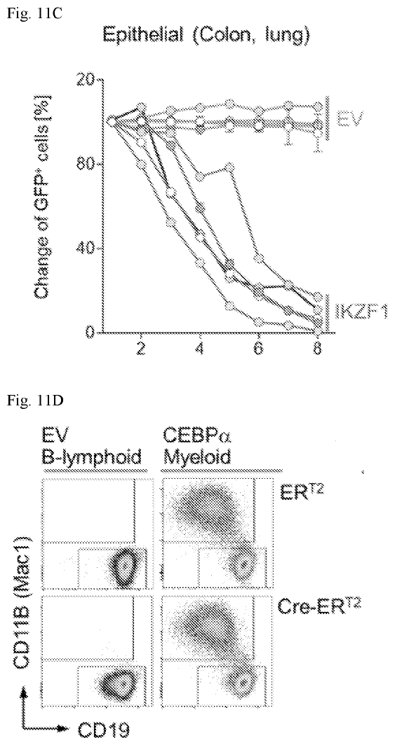

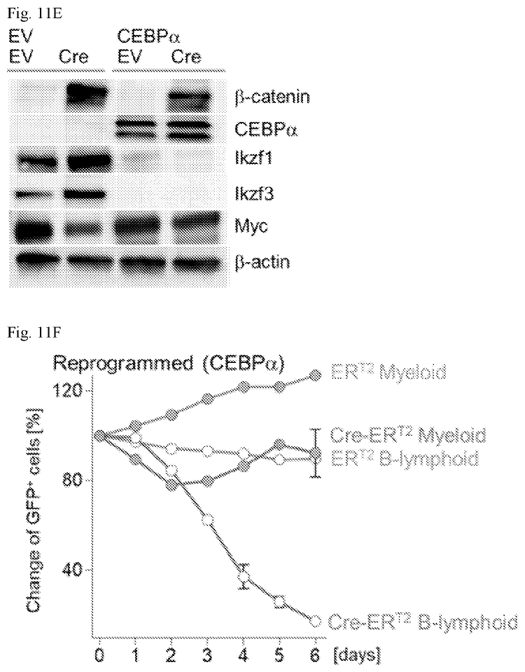

β-catenin lacks a DNA binding domain and interacts with TCF7-family transcription factors²⁻³ in epithelial, mesenchymal, neuronal, and myeloid cells to induce transcriptional activation of MYC⁴⁻⁶. Given that β-catenin-accumulation unexpectedly repressed MYC in B-lymphoid cells, β-catenin-interacting proteins in B-lymphoid, myeloid, and epithelial cell types were systematically compared. In an initial experiment, co-immunoprecipitation (Co-IP) experiments were performed and β-catenin binding proteins were identified in murine B-ALL cells by mass-spectrometry. Besides known β-catenin interacting proteins (Apc, Axin1, Gsk3β)⁷⁻⁸, the proteins with the highest enrichment of binding to β-catenin included the lymphoid-specific Ikaros transcription factors Ikaros (Ikzf1) and Aiolos (Ikzf3). These Ikaros family factors are unique to B-lymphoid cells and function as transcriptional repressors and recruit components of the repressive nucleosome remodeling and histone-deacetylase (NuRD) complex³¹⁻³⁶. Importantly, NuRD complex components (Chd4, Mta1, Mta2, Rbbp4, Gatad2a, Gatad2b, Mbd3, Hdac1, Hdac2) were identified as β-catenin-interacting proteins along with Ikzf1 and Ikzf3 (FIGS. 4A-4B). To directly compare β-catenin-interacting protein complexes between human B-lymphoid, myeloid, and epithelial cells, proteins bound to β-catenin in human B-ALL, B-cell lymphoma, myeloid, lung and colon cell lines were identified by Co—IP and mass-spectrometry. Principal component analysis of β-catenin interactomes revealed that B-lymphoid cells (B-ALL, B-cell lymphoma) were clustered together along PC1 axis and separated from myeloid and colon and lung epithelial cells (FIG. 4C). In human B-lymphoid cells, IKZF1 and IKZF3 as well as the repressive NuRD components CHD4, RBBP4, MTA1, MTA2 and GATAD2B were among the most prominent interaction partners of β-catenin (FIGS. 4D-4E, 11A). In myeloid cells, β-catenin mainly interacted with a common core module of known interaction partners including CTNNA1, AXIN2, CTNNA2 and APC, that was also shared with all other cell types studied. In colon and lung epithelial cells, β-catenin preferentially interacted with TCF7L2 (FIG. 4E) and histone acetyltransferases (KAT2B, TAF1; FIGS. 4D, 4F). Of particular interest was the epithelial cell-specific interaction of β-catenin with RUVBL1 (FIGS. 4D, 4F), which promotes β-catenin-mediated transcriptional activation of MYC³⁷⁻³⁸. Epithelial cell-specific binding of RUVBL1 to β-catenin was consistent with transcriptional activation of MYC by β-catenin-TCF7 complexes in epithelial cells⁴⁻⁶. In contrast, β-catenin induced repression of MYC in B-lymphoid cells and formed complexes with Ikaros factors and repressive NuRD components (FIGS. 4D-4F).

### Example 5. β-Catenin-NuRD Complex Interactions Depend on Lymphoid-Specific Ikaros Factors

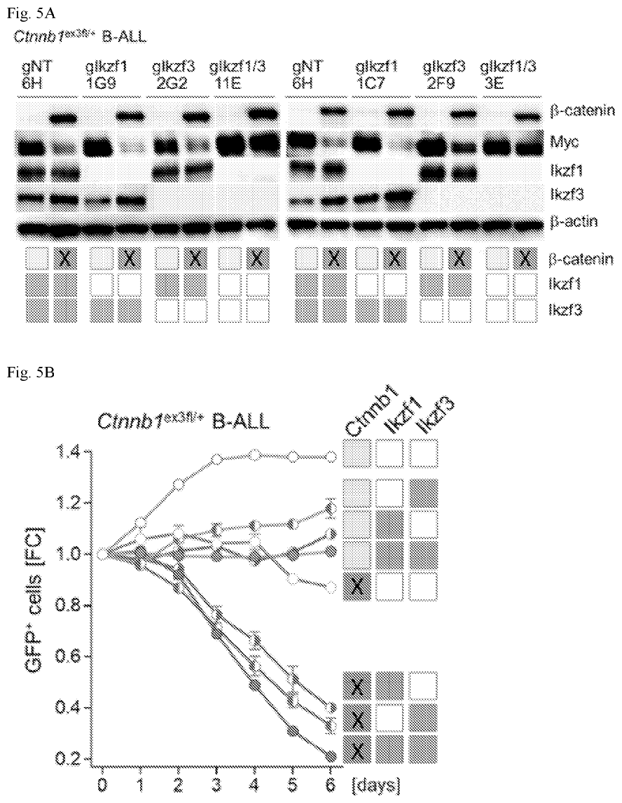

To test whether the unusual composition of the β-catenin interactome in B-lymphoid cells depends on lymphoid-specific Ikaros-factors, both Ikzf1 and Ikzf3 were deleted in murine B-ALL cells and repeated the Co-IP and mass spectrometry identification of β-catenin-interacting proteins. Deletion of Ikzf1 and Ikzf3 in B-ALL cells was achieved by electroporation-based delivery of Cas9 ribonucleoproteins (RNPs) containing Cas9 and guide-RNAs directed against Ikzf1 (glkzf1) and Ikzf3 (glkzf3) or a non-targeting control (gNT). Successful deletion was confirmed by Western blot screening of single clones for the loss of Ikzf1 and Ikzf3 (FIG. 5A). Interestingly, deletion of Ikaros-factors resulted in loss of interactions between β-catenin and the NuRD complex components Chd4, Mbd3, Mbd2, Mta2 and Gatad2b (FIG. 4G). Previous work demonstrated that loss of IKZF1 in B-ALL results in a shift from lymphoid to epithelial lineage features³². Consistent with these findings, the present results suggest that β-catenin promotes, by default, transcriptional activation of an epithelial program, unless Ikaros factors redirect β-catenin to recruit repressive NuRD complexes characteristic of B-lymphoid cells.

### Example 6. Ikaros-Factors Determine the Outcome of β-Catenin Signaling

Since expression of Ikaros-factors and β-catenin are inversely correlated in B-lymphoid and epithelial cells (FIG. 1), it was tested whether ectopic expression of IKZF1 in epithelial tumor cell lines with constitutively high β-catenin protein levels caused transcriptional repression of MYC comparable to ectopic activation of β-catenin in Ikaros-expressing B-lymphoid cells. Three colon and three lung cancer cell lines were transduced with constructs for doxycycline-inducible expression of IKZF1 or empty vector (EV) controls. Small molecule inhibition of GSK3β induced accumulation of β-catenin with slightly increased MYC expression in colon and lung cancer cell lines in the absence of IKZF1-expression. However, in the presence of ectopic IKZF1 expression, small molecule inhibition of GSK3β and accumulation of β-catenin had the opposite effect and suppressed MYC expression and induced cell death (FIGS. 11B-11C). The outcome of β-catenin activation in colon and lung cells with ectopic IKZF1 expression was the same as in B-lymphoid cells with constitutive expression of Ikaros-factors. In a converse experiment, it was determined whether the effects of β-catenin activation were dependent on B-cell identity and expression of B-lymphoid Ikaros-factors. Hence, Ctnnb1^{ex3fl/+} B-ALL cells were reprogrammed into the myeloid lineage by inducible expression of the myeloid transcription factor CEBPα. Two days after doxycycline-induced expression of CEBPα, B-ALL cells expressed the myeloid cell antigen CD11B (Mac1) and lost expression of CD19, Ikzf1 and Ikzf3 (FIGS. 11D-11E). While Cre-mediated accumulation of β-catenin abolished competitive fitness and MYC expression in B-ALL cells, CEBPα-mediated myeloid-reprogramming fully restored clonal fitness and MYC expression levels (FIGS. 11E-11F). Collectively, these findings suggest that Ikaros-factors determine the outcome of β-catenin signaling, namely transcriptional activation vs repression of MYC and other β-catenin/WNT targets. Beyond MYC, inducible accumulation of β-catenin broadly amplified Ikaros-mediated gene expression changes and deepened Ikaros-mediated repression and augmented transcriptional activation by Ikaros factors (FIG. 4H).

### Example 7. One Single Ikaros Factor is Required and Sufficient to Enable β-Catenin-Induced Repression of MYC

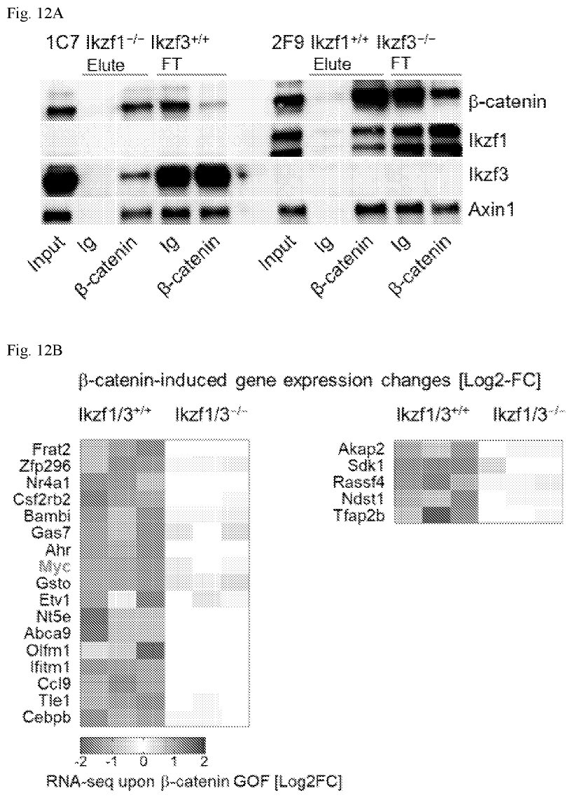

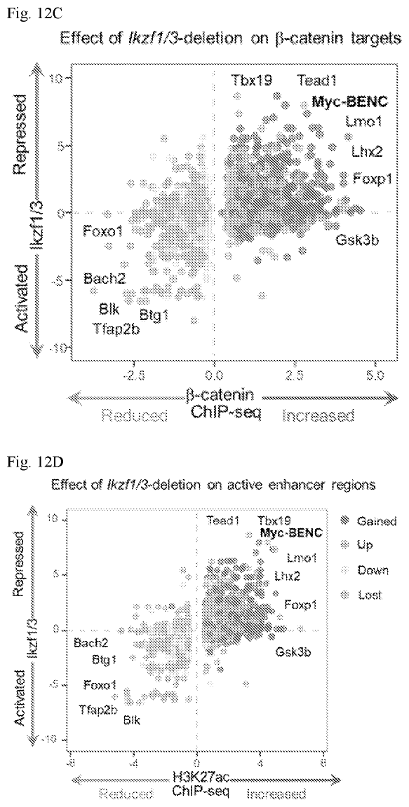

Deletion of IKZF1 is a frequent lesion in B-ALL³⁹, whereas IKZF3 mutations are common in mature B-cell malignancies⁴⁰. However, these lesions are typically monoallelic and cases with defects of both IKZF1 and IKZF3 are exceedingly rare. For this reason, it was tested whether deletion of one single Ikaros factor, either Ikzf1 or Ikzf3 alone could rescue β-catenin-induced repression of MYC, survival and proliferation of B-ALL cells. While deletion of one Ikaros factor, either Ikzf1 or Ikzf3, had no significant effects, only concurrent biallelic deletion of both B-lymphoid Ikaros factors reversed Myc-repression and cell death upon inducible accumulation of β-catenin (FIGS. 5A-5B). This result suggests that the expression of one single Ikaros factor is required and sufficient for β-catenin-induced repression of MYC and induction of cell death. Indeed, in B-ALL cells harboring a biallelic deletion of either Ikzf1 or Ikzf3, complex formation between β-catenin and the residual Ikaros factor remained intact, suggesting that heterodimerization between Ikzf1 and Ikzf3 is not required for complex formation with β-catenin (FIG. 12A). In contrast, biallelic deletion of both Ikzf1 and Ikzf3 fully rescued colony formation, cell survival in a competitive cell culture assay and restored Myc-expression and Myc-driven transcriptional programs (FIGS. 5B-5E). Measuring the effects of β-catenin-accumulation on enhancer activity (H3K27ac ChIP-seq) and gene expression (RNA-seq) in B-ALL cells, deletion of both Ikaros-factors largely erased effects of β-catenin-accumulation on gene expression, including repression of Myc (FIGS. 5C, 12B-12D). These findings suggest that β-catenin activity is mainly directed by Ikaros factors and functions as an amplifier of Ikaros-dependent gene expression changes in B-lymphoid cells. Interestingly, β-catenin accumulation had opposite effects on Myc transcriptional programs, entirely depending on whether Ikaros factors were functional (repression) or deleted (activation; FIG. 5F). In a genome-wide analysis, β-catenin accumulation suppressed enhancer activity and H3K27ac marks in the presence of functional Ikaros factors at β-catenin peaks (FIG. 5G). Upon deletion of Ikaros factors, however, accumulation of β-catenin had the opposite effect and massively increased enhancer activity (H3K27ac) at β-catenin ChIP-seq peaks (FIG. 5G). Together these findings suggest that β-catenin can, in principle, act as a powerful transcriptional activator in B-cells as in other cell types. However, B-lymphoid Ikaros factors reverse its positive effects on enhancer activity at β-catenin targets, including Myc.

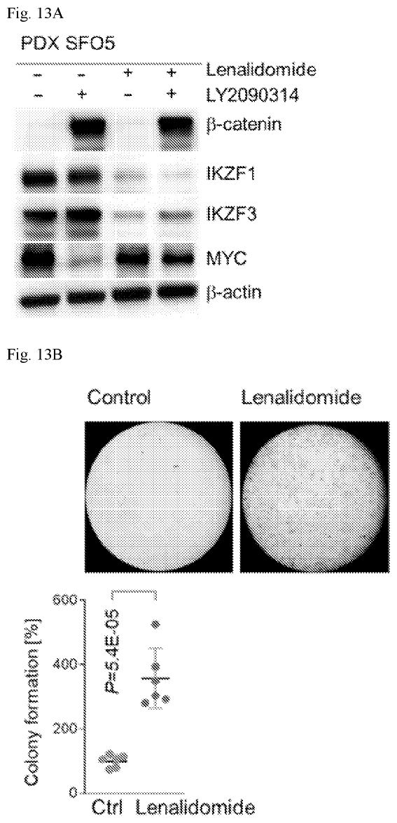

Besides genetic ablation, pharmacological degradation of IKZF1 and IKZF3 were also studied by the cereblon modifier lenalidomide. Mechanistically, lenalidomide binds to the cereblon CRBN-CRL4 ubiquitin ligase to change its substrate affinity for selective ubiquitination and degradation of IKZF1 and IKZF3 proteins⁴¹⁻⁴². Here it was shown that lenalidomide not only induced efficient degradation of both IKZF1 and IKZF3 proteins in patient-derived B-ALL cells but also relieved β-catenin-induced transcriptional repression of MYC and suppression of colony formation (FIGS. 13A-13B). Interestingly, lenalidomide treatment of patients with other hematological malignancies occasionally results in the development of B-ALL⁴³⁻⁴⁴. Hence, degradation of Ikaros-factors and derepression of MYC could be part of the underlying mechanism leading to the development- of lenalidomide-induced B-ALL in these cases.

### Example 8. Ikaros Factors Redirect β-Catenin from its Canonical TCF7 Binding Sites

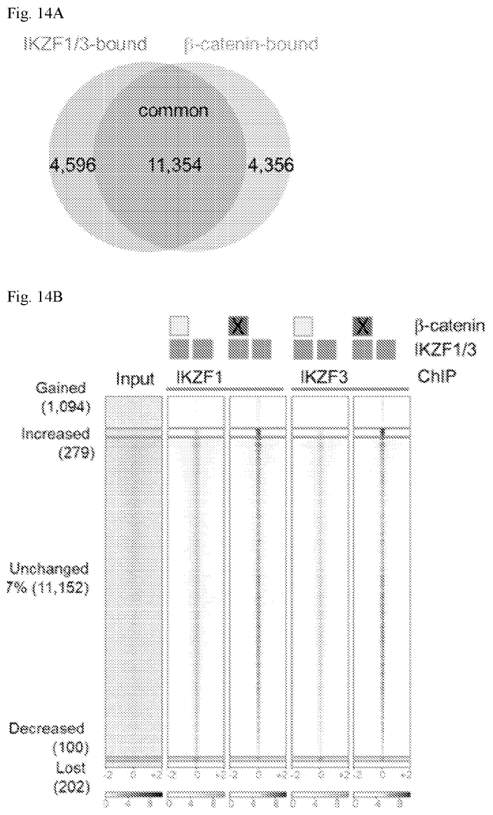

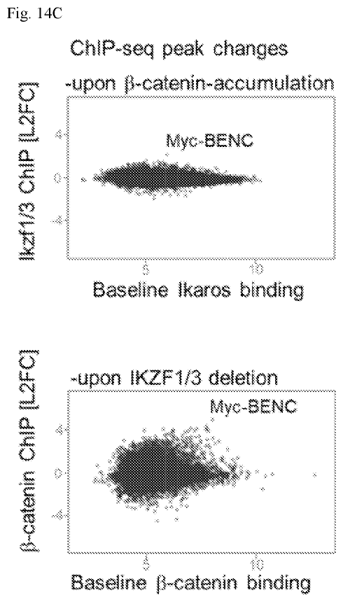

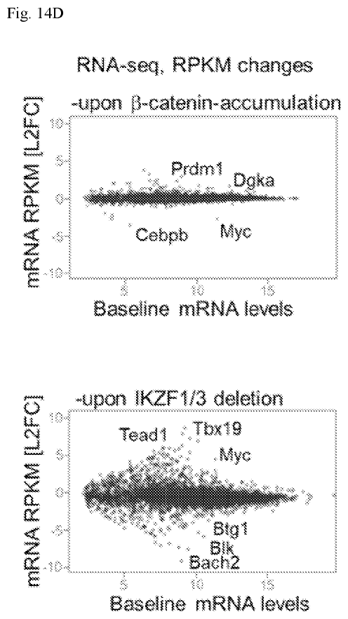

To determine how Ikaros factors and β-catenin interact at the chromatin level, β-catenin, Ikzf1 and Ikzf3 ChIP-seq analyses were performed to study changes of β-catenin- and Ikaros-binding peaks upon deletion of Ikaros factors or accumulation of β-catenin. Consistent with a dominant role of Ikaros factors in controlling β-catenin functions in B-cells, nearly 75% of β-catenin peaks were shared with Ikaros (Ikzf1, Ikzf3; FIG. 14A). Inducible accumulation of β-catenin had very limited impact on Ikzf1 and Ikzf3 binding, ~90% of Ikaros peaks remained unchanged (FIG. 14B), with peaks in a Myc superenhancer region, termed blood enhancer cluster (BENC)⁴⁵ among very few regions with increased Ikaros binding (FIG. 14C). Likewise, β-catenin accumulation caused few changes in mRNA levels, including downregulation of Myc (FIG. 14D). In contrast, loss of Ikaros-factors profoundly impacted β-catenin binding, affected about half of all β-catenin targets, generated 1,202 new β-catenin-binding peaks, while 1,675 β-catenin peaks were lost (FIG. 15A). The Myc-BENC superenhancer region was among the regions with most prominent increases of β-catenin binding and increased Myc mRNA levels upon deletion of Ikaros factors (FIGS. 14C-14D).

When β-catenin accumulation was induced in the presence of Ikaros factors, new β-catenin peaks were mostly devoid of both H3K27ac and H3K4me3 marks. However, upon deletion of Ikzf1 and Ikzf3, β-catenin peaks were substantially enriched for H3K27ac binding, suggesting increased enhancer activity at these sites. H3K4me3 marks were not changed upon Ikaros deletion (FIG. 15A). The predominant increases of H3K27ac rather than H3K4me3 marks mirrored preferential interaction of β-catenin-Ikaros complexes with NuRD complex components, whereas histone methyltransferases and demethylases were not found among β-catenin-interacting proteins (FIGS. 4D-4G). De novo β-catenin peaks associated with gain of H3K27ac marks suggest that deletion of Ikzf1 and Ikzf3 enabled redistribution of β-catenin to previously inactive enhancer regions to activate them. Motif enrichment analyses revealed that Ikaros-factor deletion restored targeting of β-catenin to classical TCF7, TCL7L1 and TCF7L2 motifs (FIG. 15A), consistent with canonical WNT signaling in epithelial cells²⁻³. These data suggest that Ikaros-factors sequester β-catenin away from transcriptional activation at canonical TCF7 sites. Consistent with a scenario in which Ikaros factors interfere with canonical β-catenin-TCF7 interactions in B-lymphoid cells, Ikaros factor deletion enabled or increased complex formation of β-catenin with TCF7-family transcription factors (Tcf7, Tcf711, Tcf712; FIGS. 15D-15C).

### Example 9. IKZF-Factors in Lymphoid Cells Displace Transcriptional Foxp1/TCF7L2 Coactivators of β-Catenin

Comparing β-catenin-interacting proteins in the presence and absence of dual Ikzf1 3 deletion, it was found that upon loss of IKZF-factors, β-catenin no longer associated with repressive NuRD components Chd4, Gatad2b and Mta2 and instead formed complexes with the transcriptional coactivator of WNT/β-catenin signaling Foxp1 (FIG. 22A). The newly formed complexes between β-catenin and Foxp1 in Ikzf1 3-deficient B-ALL cells are of particular interest: previous work demonstrated frequent copy number amplifications and chromosomal translocations resulting in FOXP1-gain of function in mature B-cell lymphomas³⁰. Thereby, FOXP1 forms a complex with both β-catenin and TCF7L2 to promote aberrant WNT/β-catenin signaling in B-cell lymphoma³⁰. This is seemingly at odds with the finding that lymphoid cells did not tolerate oncogenic β-catenin signaling (FIGS. 1; 2; 19; 20). For this reason, the effects of forced overexpression of Foxp1 were examined, similar to gain-of-function lesions in B-cell lymphoma. While β-catenin activation alone resulted in rapid B-ALL cell death, overexpression of Foxp1 largely rendered B-ALL cells permissive to β-catenin hyperactivation (FIG. 22B). These results support a scenario in which IKZF-factors in lymphoid cells assemble repressive NuRD components to displace FoxpL.TCF7 complexes. Forced overexpression of FOXP1 outcompetes IKZF factors and enables transcriptional activation downstream of oncogenic WNT/β-catenin signaling, as in B-cell lymphomas that carry gain-of-function lesions of FOXP1 (FIG. 15C).

### Example 10. β-Catenin is Required for Ikaros-Mediated Tumor Suppression

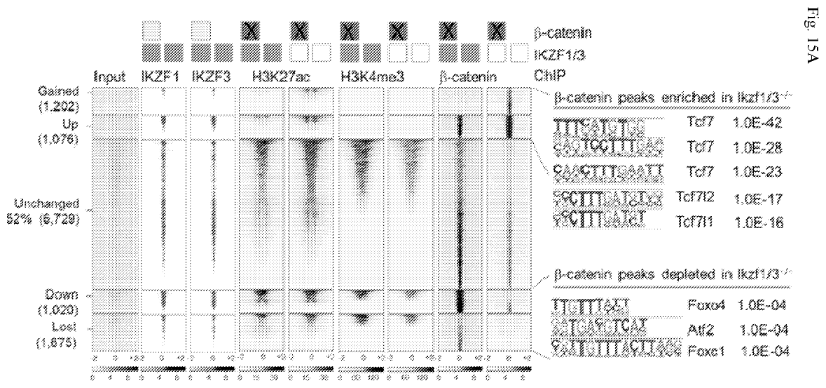

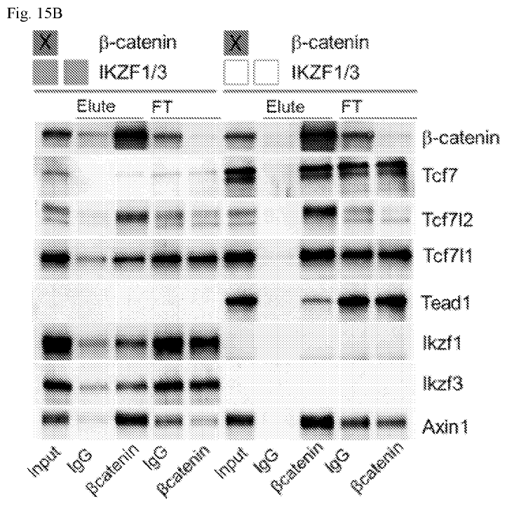

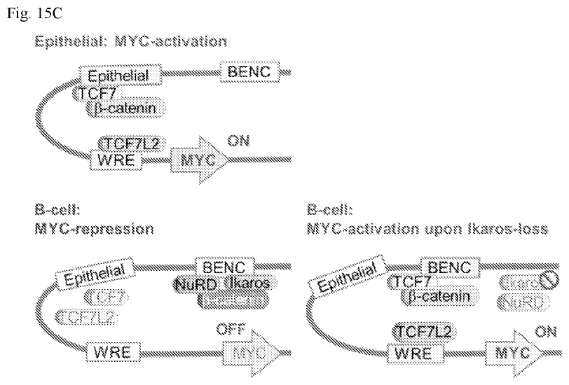

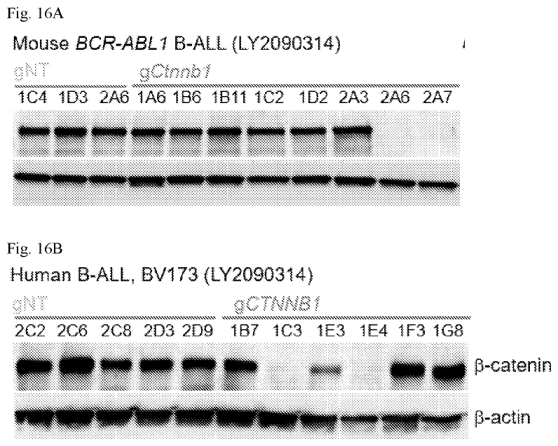

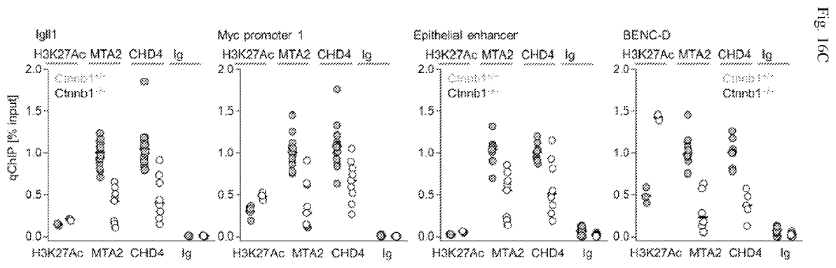

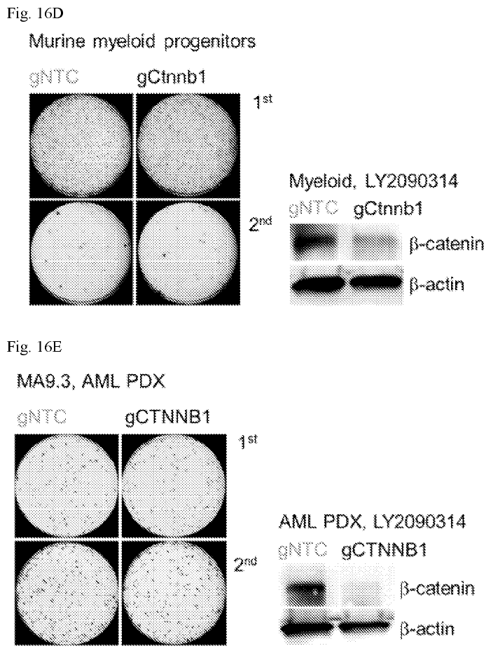

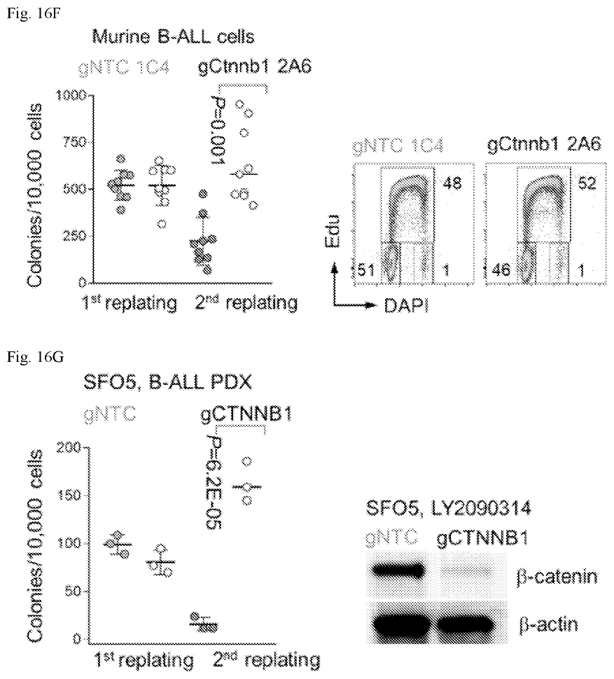

The B-lymphoid Ikaros family factors IKZF1 and IKZF3 function as important tumor suppressors in B-ALL³⁹ and mature B-cell malignancies⁴⁰, respectively. Unlike epithelial, myeloid, and T-cell lineage cells, B-lymphoid cells exhibited very low β-catenin protein expression and activity (FIGS. 1, 8, 9). Since β-catenin bound to NuRD complex components in an Ikaros-dependent manner and intensified Ikaros-dependent gene expression changes (FIGS. 4G-4H), it was examined whether β-catenin, despite its low baseline expression levels, contributes to the recruitment of components of repressive NuRD complexes and regulation of enhancer activity at β-catenin/Ikaros target loci. To this end, genetic deletion of β-catenin was engineered in murine and human B-ALL cells and validated successful β-catenin deletion for single-cell clones by Western blot (FIGS. 16A-16B). Genetic deletion of β-catenin did not significantly change enhancer activity (H3K27ac) at Igll1, Myc promoter and Myc epithelial enhancer regions. However, β-catenin-deletion markedly increased H3K27ac levels at BENC⁴⁵ Myc superenhancer regions C and D (FIGS. 5H, 16C). Conversely, deletion of β-catenin modestly reduced recruitment of repressive NuRD complex components MTA2 and CHD4 at Igll1, Myc promoter and Myc epithelial enhancer regions, compared to near-complete loss of MTA2 and CHD4 recruitment at Myc superenhancer regions BENC-C and BENC-D (FIGS. 5H, 16C). Mirroring B-cell-specific functions of β-catenin-Ikaros complexes, loss of β-catenin significantly improved colony formation of β-lymphoid but not myeloid progenitor cells of human and murine origin (FIGS. 5I, 16D-16G). Of note, this difference only became apparent in secondary replating experiments. Consistent with a role of β-catenin in negatively regulating B-cell leukemia-initiation in transplant experiments (FIGS. 2F-2G), these results suggest that β-catenin-Ikaros complexes primarily limit self-renewal of B-lymphoid cells. To directly test a role of β-catenin in Ikaros-mediated tumor suppression, the effect of inducible activation of IKZF1 in B-ALL cells was compared with intact β-catenin (gNT) and B-ALL cells with genetic deletion of β-catenin (gCtnnb1). While IKZF1-induction induced cell death and suppressed proliferation in gNT B-ALL cells, deletion of β-catenin subverted IKZF1-mediated tumor suppression in gCtnnb1 B-ALL cells (FIG. 5J). These results imply that despite low baseline expression levels, β-catenin, and its ability to engage Ikaros factors for the recruitment of repressive Ikaros:NuRD complexes represents a critical and previously unrecognized tumor suppressor in B-lymphoid malignancies.

### Example 11. Both β-Catenin and Ikaros Factors are Required for Efficient NuRD Complex Recruitment

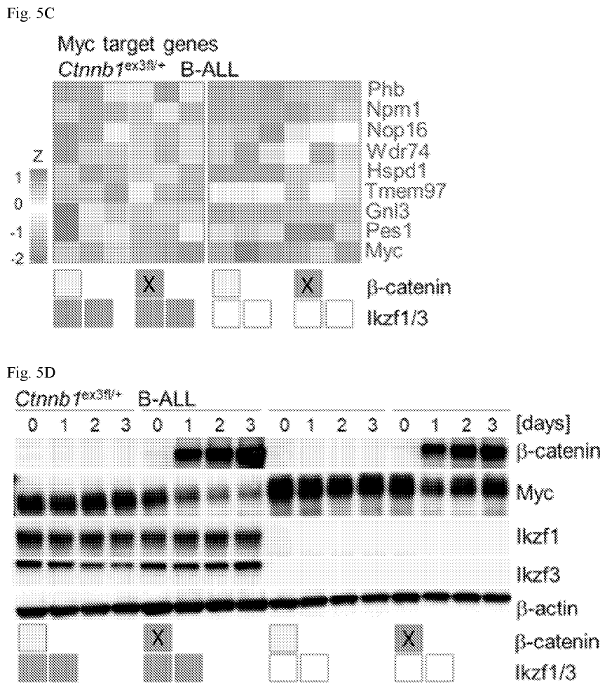

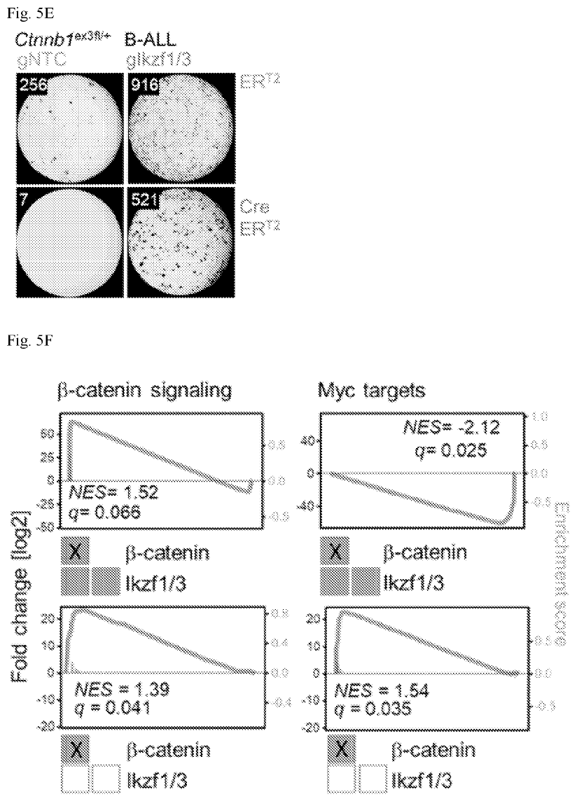

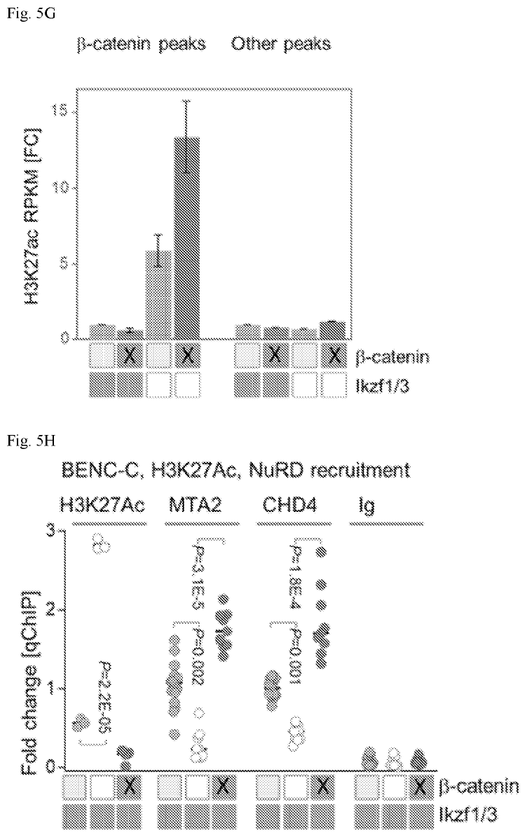

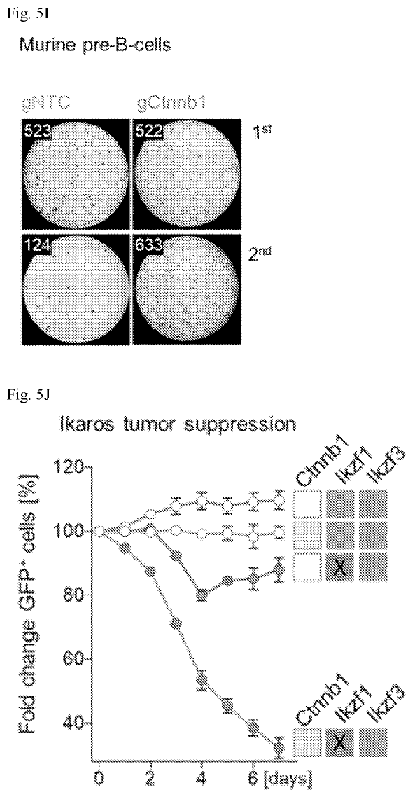

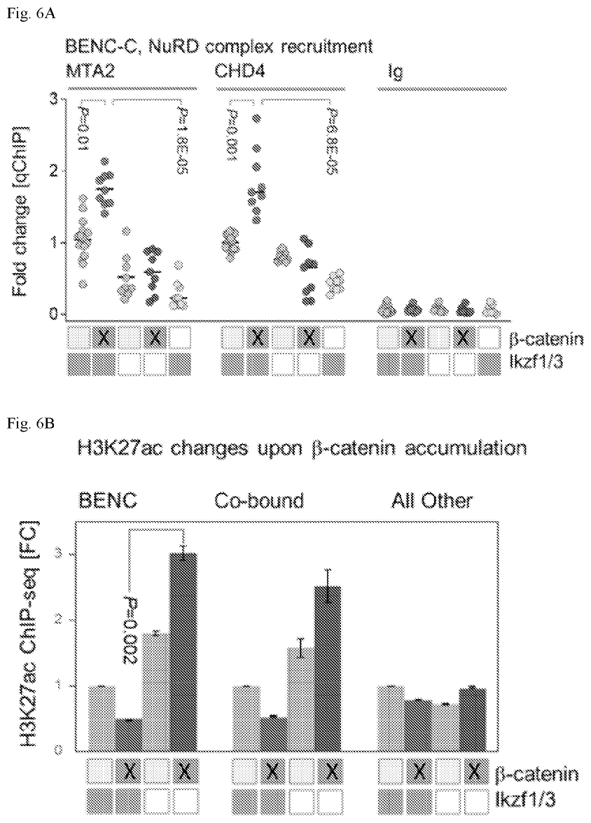

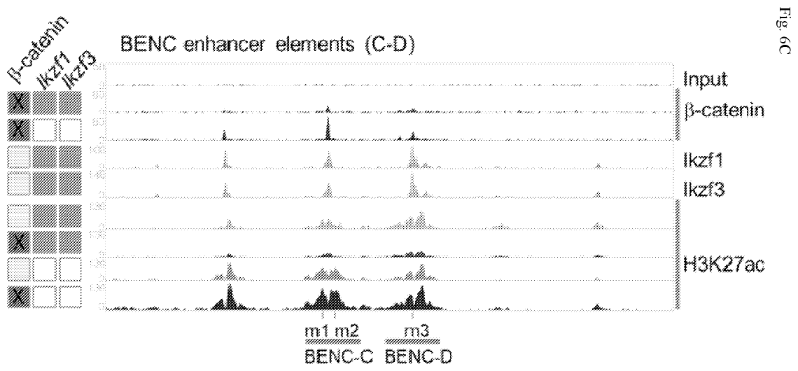

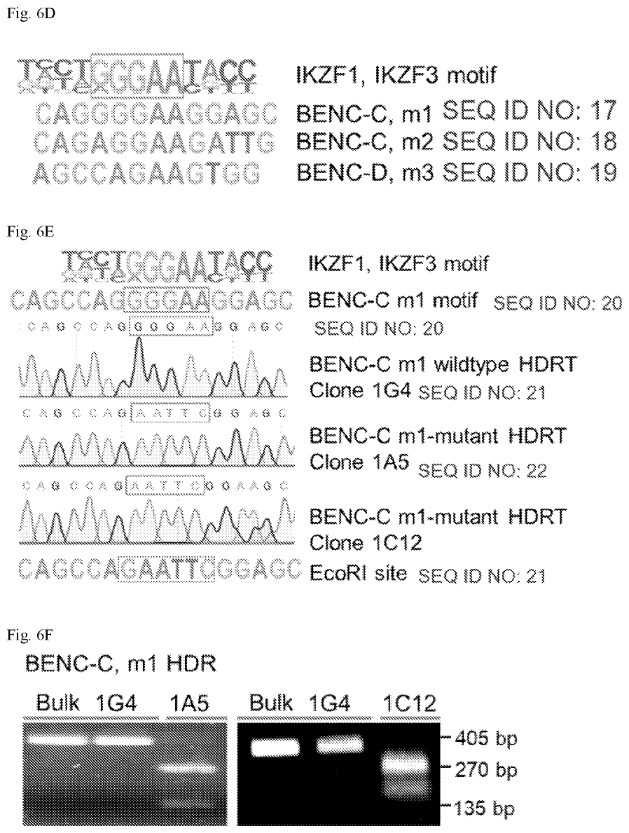

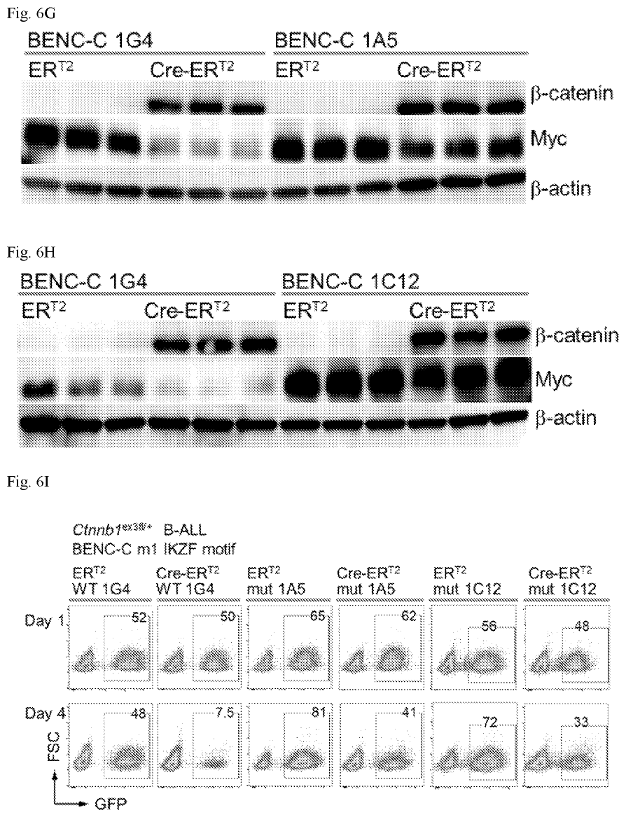

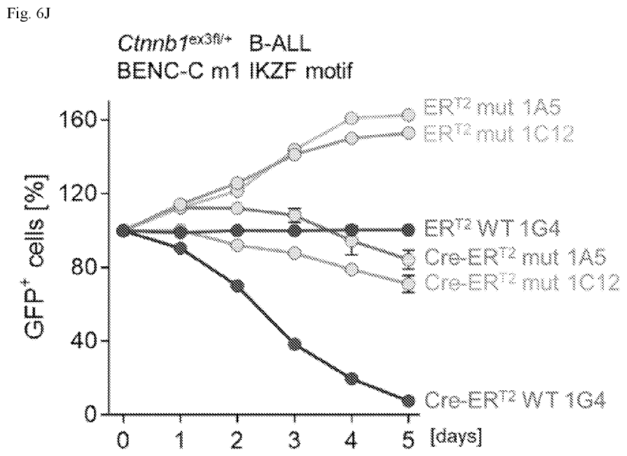

Deletion of Ikaros factors disrupted interactions between β-catenin and NuRD complex components (FIG. 4G) and relieved β-catenin-induced repression of Myc (FIGS. 5C-5D, 5F). Since β-catenin is required for effective NuRD complex recruitment (FIGS. 5H, 16C) and Ikaros-mediated tumor suppression (FIG. 5J), it was tested whether β-catenin and Ikaros factors cooperate in recruiting NuRD complex components. Consistent with cooperation between β-catenin and Ikaros factors, recruitment of the NuRD components MTA2 and CHD4 to BENC enhancer regions of Myc was increased by accumulation of β-catenin, but nearly entirely lost upon deletion of Ikaros factors or deletion of β-catenin (FIGS. 6A, 17). In addition to BENC Myc enhancer regions, deletion of Ikaros and β-catenin also affected NuRD complex recruitment at the Myc promoter and the Ikaros target gene Igll1 but not epithelial Myc enhancer regions (FIG. 17A). Collectively, these results suggest that both Ikaros and β-catenin are required for effective NuRD complex recruitment and transcriptional repression of Myc in B-lymphoid cells.

### Example 12. IKZF1 and IKZF3 Mediate Transcriptional Repression of MYC at BENC Superenhancer Regions

Studying H3K27ac signals across multiple MYC super-enhancer clusters revealed that most of the enhancer activity was concentrated in blood enhancer cluster (BENC) regions (FIG. 17B), which was identified as critical for the regulation of MYC expression in B-lymphoid and other hematopoietic cells⁴⁵. Consistent with predominant recruitment of NuRD complex components at these regions (FIGS. 6A, 17A), Ikaros factors and β-catenin strongly bound to elements C-D of the BENC region (FIGS. 6B-6C, 17B). In the presence of Ikaros factors, β-catenin-accumulation suppressed H3K27ac signals at BENC regions. However, in the absence of Ikaros factors, β-catenin- accumulation had the opposite effect and significantly increased H3K27ac signals at BENC enhancer regions (FIGS. 6B-6C, 17B). Interestingly, other loci that were bound by both Ikaros factors and β-catenin showed a similar pattern (FIG. 6B). While β-catenin-Ikaros complexes suppressed MYC-expression in B-lymphoid cells, these observations suggest that deletion of Ikzf1 and Ikzf3 releases β-catenin from transcriptional repression and restores its ability to promote transcriptional activation of MYC as in non-lymphoid cell types⁴⁻⁶.

### Example 13. Pharmacological Activation of β-Catenin to Engage Repressive IKZF Complexes in Lymphoid Malignancies

Sequence analysis of BENC-C and BENC-D regions identified three Ikaros binding motifs (m1-m3) at significant Ikzf1 and Ikzf3 ChIP-seq peaks (FIGS. 6C-6D). Of these, m1 perfectly matched the Ikaros motif (GGGAA), whereas the other two had a single base pair mismatch. To test the functional significance of the Ikaros m1 motif within the Myc BENC-C superenhancer region, knockin alleles were engineered to replace the Ikaros binding motif with an EcoRI site. After HDRT-based knockin of wildtype and mutant BENC-C alleles into murine Ctnnb1^{ex3fl/+} B-ALL cells, clones carrying the knockin mutation were selected based on EcoRI digestion and confirmed by Sanger sequencing (FIGS. 6E-6F). Ctnnble^{x3fl/+} B-ALL clones with wildtype and mutant BENC-C Ikaros motifs were transduced with inducible Cre for accumulation of β-catenin. As expected, BENC-C wildtype knockin clones rapidly lost Myc expression and underwent cell death upon inducible accumulation of β-catenin (FIGS. 6G-6J). In contrast, Ctnnb1^{ex3fl/+} B-ALL clones carrying knockin alleles for the mutant Ikaros m1 motif in BENC-C expressed Myc at higher baseline levels and were resistant to inducible β-catenin accumulation. Upon β-catenin accumulation, Myc levels remained high. Cell viability and competitive fitness of B-ALL clones carrying the mutant Ikaros m1 motif remained largely unchanged (FIGS. 6G-6J). While it is likely that β-catenin accumulation has other effects in B-ALL cells, these findings underscore that β-catenin-induced toxicity and repression of Myc primarily depend on its interactions with Ikaros factors and in particular one single Ikaros binding site within the Myc BENC-C enhancer region.

### Example 14. Pharmacological Activation of β-Catenin-Ikaros Complexes for Refractory B-Cell Malignancies

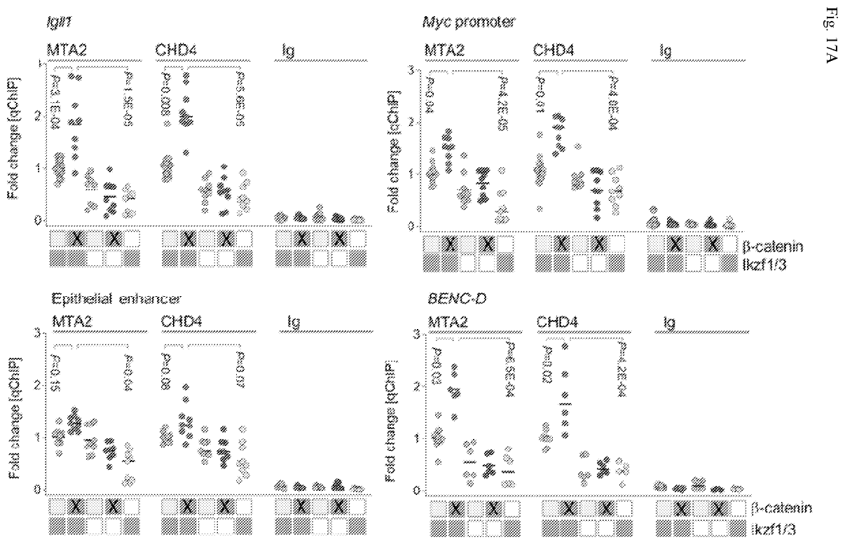

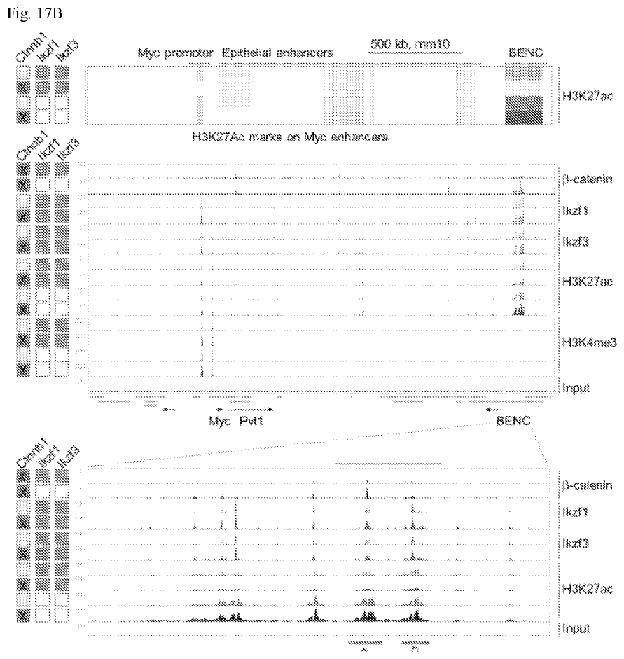

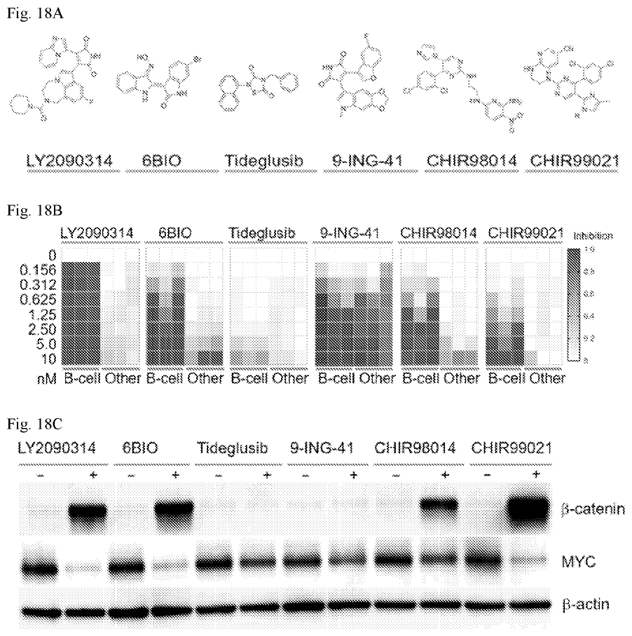

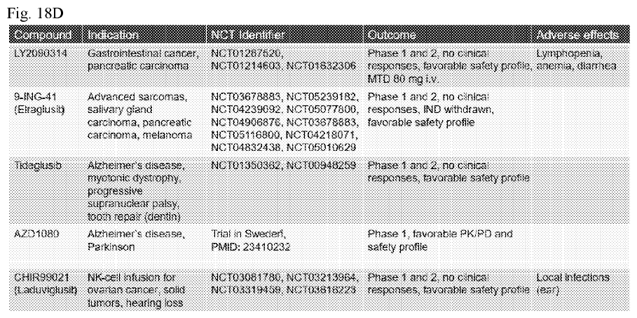

Given that low baseline expression levels of β-catenin were sufficient to enable tumor suppression by Ikaros-factors in B-ALL cells (FIG. 5J), pharmacological accumulation of β-catenin could potentiate tumor suppressive effects of β-catenin-Ikaros complexes. The present genetic approaches achieved β-catenin accumulation based on Cre-mediated excision of GSK3β-phosphorylation sites³⁰. This previously unrecognized strategy to engage β-catenin-Ikaros complexes would be orthogonal to conventional mechanisms of drug-resistance and potentially useful in the treatment of patients with relapsed or refractory B-cell malignancies. For this reason, pharmacological approaches of β-catenin accumulation were next tested based on small molecule inhibitors of GSK3β. To address potential safety concerns related to pharmacological β-catenin accumulation, the present analysis was focused on compounds that have completed clinical development and demonstrated favorable safety profiles in clinical trials (FIG. 18).

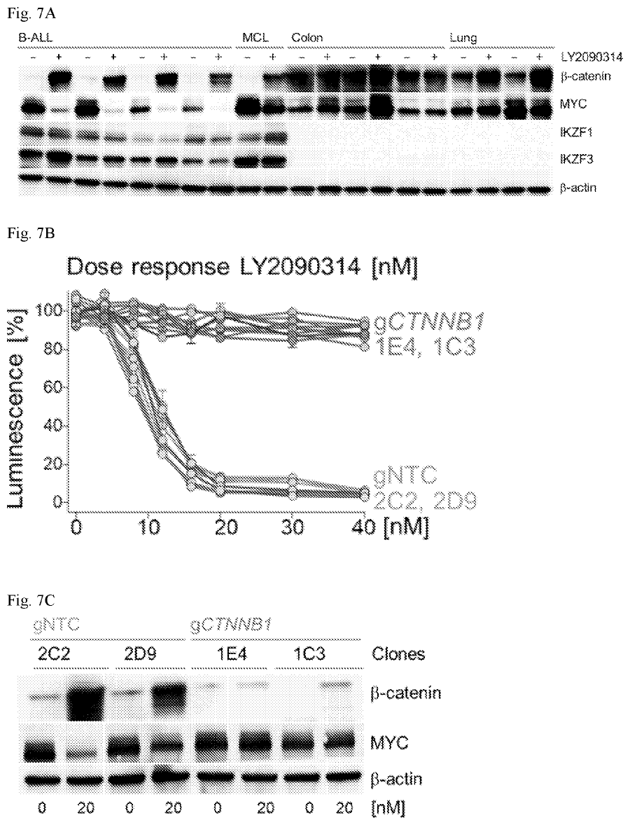

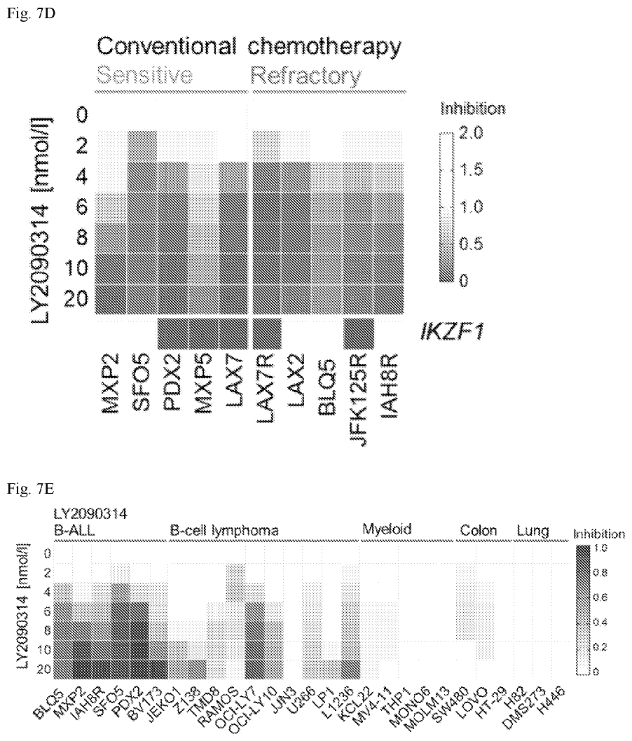

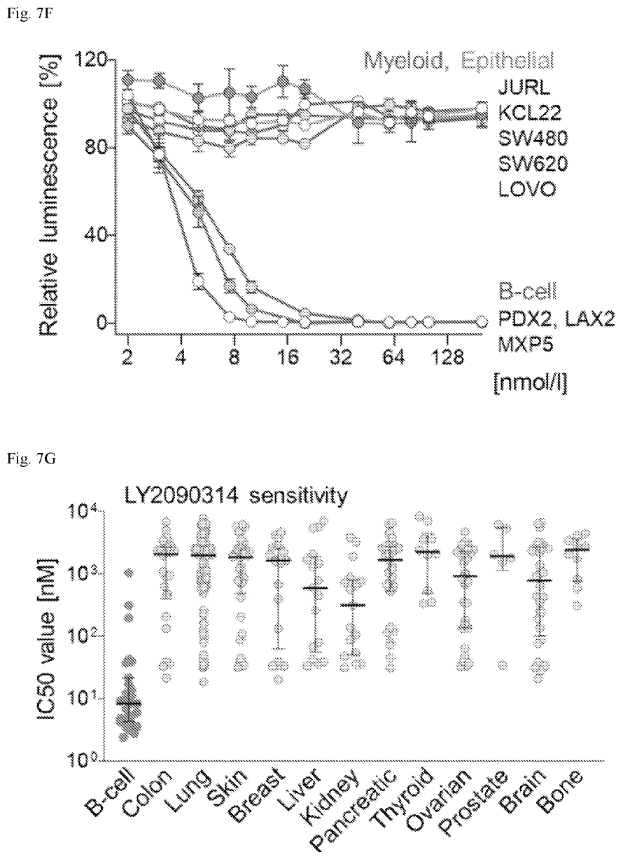

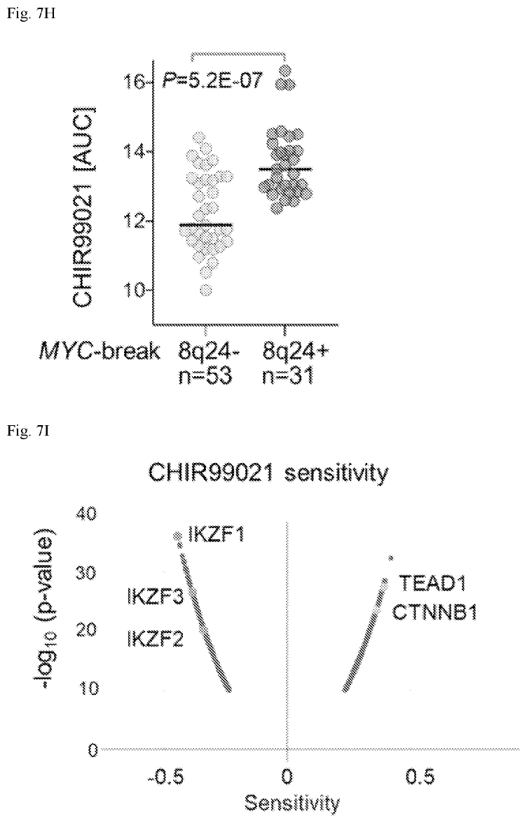

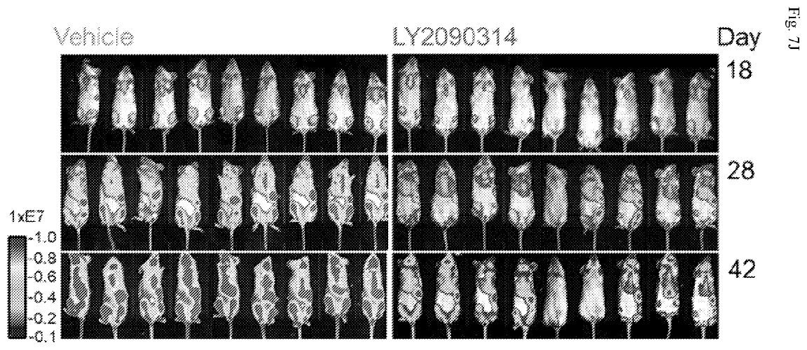

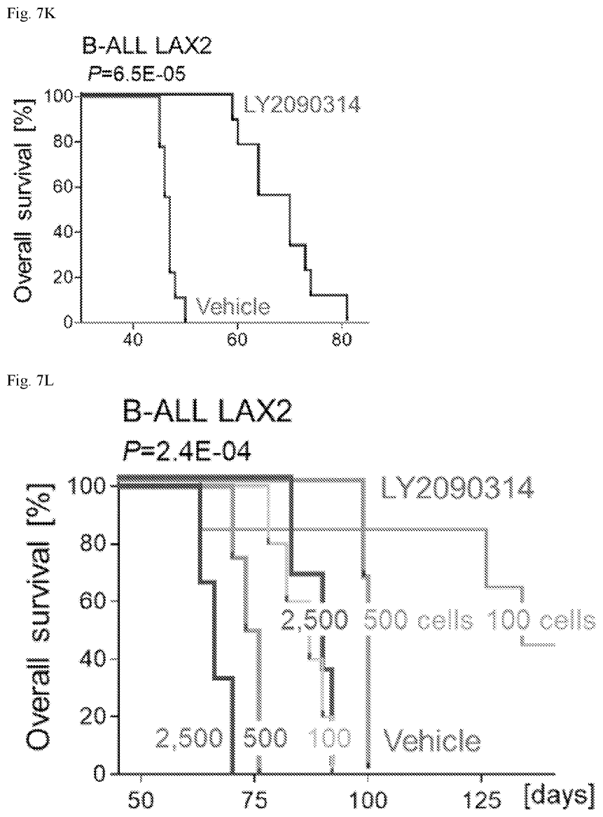

For proof-of-concept studies, six small molecule GSK3β inhibitors were tested for their ability to selectively kill B-lymphoid leukemia and lymphoma cells. Four of the six GSK3β inhibitors (LY2090314, 6-Bromoindirubin-3′-oxime, CHIR98014 and CHIR99021) induced cell death at low nanomolar concentrations selectively in B-lymphoid but not myeloid and epithelial cells (FIGS. 18A-18B). In contrast, Tideglusib had no significant activity in any cell type, while 9-ING-41 showed broad non-specific toxicity across all cell types tested (FIGS. 18A-18B). Interestingly, the four GSK3β3 inhibitors with B-cell-selective toxicity (LY2090314, 6-Bromoindirubin-3′-oxime, CHIR98014 and CHIR99021) induced massive accumulation of β-catenin in parallel with acute suppression of MYC (FIGS. 7A, 7C, 18C). In contrast, lack of specific drug-responses for 9-ING-41 and Tideglusib was mirrored by failure to induce β-catenin accumulation and MYC-suppression (FIG. 18C). These results suggest that accumulation of β-catenin and MYC-suppression not only represent important biomarkers for drug-responses to GSK3β-inhibitors but also reflect their underlying mechanism of action.

### Example 15. β-Catenin Accumulation Represents the Mechanism of Action of GSK3β-Inhibitors in B-Cell Malignancies

To determine if accumulation of β-catenin indeed represents the mechanistic basis of LY2090314-mediated cell death in B-ALL cells, CTNNB1 was deleted in human B-ALL cells using Cas9-RNPs and screening of clones for CTNNB1-deletion from single cells (FIG. 16B). Reminiscent of knockin mutation of the m1 Ikaros binding motif within the Myc BENC-C superenhancer region (FIGS. 6G-6J), deletion of CTNNB1 conferred near-complete resistance of B-ALL cells to LY2090314 and prevented suppression of MYC (FIGS. 7B-7C). In the B-ALL mouse model, was shown that one single Ikaros factor (IKZF1 or IKZF3) was sufficient to suppress MYC and induce cell death upon β-catenin accumulation (FIGS. 5A-5B). Since IKZF1-deletions are common in human B-ALL³⁹, the impact of IKZF1-deletion on sensitivity to the GSK3β inhibitor LY2090314 was tested. Studying 10 patient-derived xenografts (PDX), including 5 with IKZF1-deletion, no significant differences were found in responses to LY2090314 (FIG. 7D). In addition, whether or not B-ALL PDX were derived from patients who responded to standard chemotherapy or were refractory and relapsed, did not affect responses to LY2090314. This result suggests that pharmacological β-catenin accumulation by GSK3β-inhibition is indeed orthogonal to conventional mechanisms of drug-resistance and may represent a vulnerability that could be impactful for patients with drug-resistant or relapsed B-cell malignancies.

Focused analyses of drug-responses for LY2090314 in a larger panel of cell lines and PDX corroborated profound responses in B-ALL and B-cell lymphoma cells in the absence of significant effects on myeloid and epithelial tumor cells (FIGS. 7E-7F). A combined analysis of responses to LY2090314 based on 343 epithelial cancer cell lines (Prism Drug Repurposing Secondary Screen)⁴⁶ and B-lymphoid cell lines (17 B-ALL, 7 B-cell lymphoma) revealed that IC₅₀ values for LY2090314 were substantially higher in epithelial and myeloid cancer cells than in B-ALL (236-fold) and B-cell lymphoma (92-fold; FIG. 7G). While B-ALL cell lines were uniformly sensitive, sensitivity to the GSK3β-inhibitor CHIR99021 showed a bimodal distribution across 84 B-cell lymphoma cell lines. The difference between the two groups largely tracked with presence or absence of MYC translocations: B-cell lymphoma cell lines with MYC-rearrangement (n=31; 8q24+) were substantially less sensitive to GSK3β-inhibition compared to cell lines without MYC-translocation (n=53; P=5.2E-07; FIG. 7H). Computational analyses of additional biomarkers for responses to GSK3β-inhibition across Prism panel of cell lines identified Ikaros factor (IKZF1, IKZF3, IKZF2) expression as the top-ranking association, while high baseline levels of β-catenin and the epithelial cell transcriptional factor TEAD1 showed the strongest negative correlation with sensitivity to CHIR99021 (FIG. 7I).

### Example 16. Preclinical Validation of GSK3β Inhibition for Refractory B-Lymphoid Leukemia

Five GSK3β-inhibitors, including LY2090314 and CHIR99021, had previously achieved favorable safety and PK/PD profiles at micromolar plasma concentrations (Cmax) in 22 clinical trials for patients with pancreatic cancer, advanced sarcoma, Alzheimer's disease, progressive supranuclear palsy, amyotrophic lateral sclerosis, tooth repair, hearing loss and NK-cell stimulation for immunotherapy (FIG. 18D). Since LY2090314 and CHIR99021 demonstrated B-cell selective activity at low nanomolar concentrations (FIGS. 7E-7F, 18B), accumulation of β-catenin and suppression of MYC (FIGS. 7A, 18C), a potential rationale for repurposing GSK3β small molecule inhibition towards refractory B-cell malignancies was tested. To this end, immunodeficient mice, bearing patient-derived xenografts (PDX) from refractory B-ALL cells were treated with LY2090314 as a single agent. Sublethally irradiated (2 Gy) NSG mice bearing refractory B-ALL PDX were injected intraperitoneally with LY2090314 or vehicle control for six times. Compared to mice treated with vehicle, LY2090314 substantially reduced leukemia burden and significantly extended overall survival (P=6.5E-05, n=9; FIGS. 7J-7K). Consistent with highly B-cell-selective effects of GSK3β-inhibition, treated mice did not show significant weight loss or other dose-limiting toxicity. These findings suggest that GSK3β-inhibition, when used in combination with existing regimen for refractory B-ALL and other lymphoid malignancies, could substantially deepen treatment responses and overcome mechanisms of conventional drug resistance. GSK3β small molecule inhibitors, including LY2090314, AZD1080, Laduviglusib (CHIR99021), Tideglusib and Elraglusib (9-ING-41), have demonstrated safety and tolerability at micromolar plasma concentrations (Cmax)⁴⁷⁻⁵³. Among the reported adverse effects of LY2090314 in clinical trials (Cmax micromolar) was lymphopenia (FIG. 18D), which is consistent with the unique dependency of B-lymphoid cells on GSK3β-mediated degradation of β-catenin discovered in this study. Importantly, the present studies of LY2090314 and Laduviglusib (CHIR99021) in refractory B-cell malignancies demonstrated B-cell selective effects at low nanomolar concentrations in vitro (FIGS. 7B, 7D-7G, 18B-18C) and in vivo (FIGS. 7J-7L). Consistent with impairment of B-cell leukemia-initiation upon genetic accumulation of β-catenin (FIGS. 2F-2G), pharmacological β-catenin accumulation by LY2090314 reduced the leukemia-initiation potential of B-ALL PDX in transplant recipient mice (FIG. 7L).

### Example 17. β-Catenin-Ikaros Complexes in T-Cells

Of note, loss of LmbrIl resulted in β-catenin accumulation and profound defects of both B- and T-lymphopoiesis. This would be consistent with expression and activity of some Ikaros factors (e.g. IKZF1) in both B- and T-lymphoid cells⁵⁵. However, unlike B-lymphoid cells, T-cells exhibit substantial baseline activity of β-catenin signaling (FIGS. 1C, 8) and T-cell malignancies carried activating β-catenin mutations at similar frequencies as in solid tumors (Table 8). In some T-cell malignancies, oncogenic activation of Notch1 counteracts Ikaros-mediated tumor suppression⁵⁶, which could provide a mechanism for T-lymphoid cells to become permissive to β-catenin accumulation. Seemingly contrasting the present scenario that β-catenin-Ikaros complexes suppresses lymphoid development, targeted overexpression of β-catenin in thymocytes resulted in the development of T-lymphoid malignancies^{25, 57} Strikingly, karyotypic analyses of 18 β-catenin-driven T-cell lymphomas in two studies revealed that 17 of them carried a Myc-rearrangement^{25, 57}. While Myc is a target of transcriptional activation by β-catenin in other cell types⁴⁻⁶, these results suggest that β-catenin itself did not promote Myc expression in T-cells and instead imposed selective pressure for secondary genetic lesions resulting in Myc-overexpression: In the first study, all 8 β-catenin-driven T-cell lymphomas harbored a Myc translocation, including Myc-Tcra (n=6) and Myc-Tcrb (n=1) rearrangements²⁵. In the second study, 9 of 10 β-catenin-driven T-cell lymphomas carried either a Myc-Tcra (n=3) rearrangement or large deletions downstream of Myc encompassing the BENC region (n=6)⁵⁷. The remaining case without Myc abnormality showed aberrant overexpression of N-Myc⁵⁷. Consistent with the present finding that targeted mutation of an Ikaros motif in the Myc BENC-C region conferred resistance to β-catenin accumulation (FIG. 6) and that MYC-translocations in B-cell lymphomas are correlated with reduced sensitivity to GSK3β-inhibition (FIG. 7H), these findings in T-cell malignancies^{25, 57} raise the possibility that β-catenin may form similar complexes with Ikaros factors for repression of Myc in T-cells. Studies to explore β-catenin-Ikaros complexes and the role of Myc BENC enhancer regions in normal T-cell development and T-cell malignancies are currently underway.Asbsd.

### Example 18. Limitations of GSK3β-Inhibition for the Treatment of Lymphoid Malignancies

In agreement with an early study suggesting that Lefl/β-catenin signaling may negatively regulate Myc expression in B-cells⁵⁸-as opposed to other cell types⁴⁻⁶, it was proposed herein to leverage clinically approved GSK3β-inhibitors to engage β-catenin-Ikaros complexes for targeted repression of Myc as a previously unrecognized strategy to overcome drug resistance in refractory B-cell malignancies.

Translocations of the MYC gene at 8q24 occur in about 15% of all B-cell malignancies⁵⁹⁻⁶⁰. In many of these cases, expression of translocated MYC is driven by the IGH Eμ enhancer and IGH 3′ regulatory regions and no longer regulated by its transcriptional control elements (e.g., BENC-C). The present experiments based on CRISPR-based knockin mutation of a single Ikaros-binding motif identified the lymphoid Myc BENC-C superenhancer region as a central mechanistic element in β-catenin-dependent Myc repression (FIG. 6). On this basis, it was predicted herein that GSK3β-inhibition in B-cell lymphomas with MYC translocation will likely fail to repress MYC. Consistent with this scenario, B-cell lymphoma cell lines carrying a MYC-translocation at 8q24 were substantially less sensitive to GSK3β-inhibition (FIG. 7H). These findings are in line with a previous study of a genetic mouse B-cell lymphoma model for overexpression of MYC from the IGH Ep-enhancer, which enabled secondary lesions resulting in β-catenin-hyperactivation⁶¹. In the present survey of 2,137 B-cell malignancies, 17 cases with an activating β-catenin pathway lesion were found (FIG. 1B, Table 8). Among 1,980 B-cell lymphomas with informative MYC-status and without β-catenin pathway lesion, 208 carried a MYC break at 8q24 (11%). Reminiscent of mouse models with T-cell-specific overexpression of β-catenin^{25,27}, among 14 cases with β-catenin pathway lesions and informative MYC-status, 12 carried a MYC-break (expected 1.5, observed 12, x test P=3.6 E-5; Table 9).

TABLE 9
Cooccurrence of MYC translocations with B-catenin-pathway lesions in β-cell malignancies

| | Sample | Sample | AA | CDS | | MYC- | Pubmed | CGP | |
|---|---|---|---|---|---|---|---|---|---|
| Gene | Name | ID | Mutation | Mutation | Histology | break | Id | Study | Genomic GRCh38 |
| CTNNB1 | E13453 | 740574 | p.D32H | c.94G > C | DLBCL | Unknown | 11927008 | — | 3: 41224606 . . . 41224606 |
| CTNNB1 | QC2-39-T2 | 2462817 | p.A21D | c.62C > A | DLBCL | Yes | 26647218 | — | 3: 41224574 . . . 41224574 |
| APC | tumor_4119027 | 1819096 | p.? | c.646 − | Burkitt's | Yes | — | COSU440 | 5: 112790314 . . . 112790314 |
| | | | | 2132T > G | | | | | |
| APC | tumor_4182393 | 1819101 | p.? | c.136 − | Burkitt's | Yes | — | COSU440 | 5: 112760786 . . . 112760786 |
| | | | | 5540T > C | | | | | |
| APC | tumor_4189998 | 1819102 | p.G2227V | c.6680G > T | Burkitt's | Yes | | | |
| APC | 29_tFL | 2225699 | p.R303K | c.908G > A | DLBCL | Yes | 24388756 | — | 5: 112815568 . . . 112815568 |
| APC | QC2-39-T2 | 2462817 | p.E2074K | c.6220G > A | DLBCL | Yes | 26647218 | — | 5: 112841814 . . . 112841814 |
| APC | P566 | 2878355 | p.C575* | c.1725T > A | DLBCL | No | 32187361 | — | 5: 112828954 . . . 112828954 |
| APC | P639 | 2878428 | p.R348* | c.1042C > T | DLBCL | Unknown | 32187361 | — | 5: 112819074 . . . 112819074 |
| APC | P890 | 2878679 | p.Q1477* | c.4429C > T | DLBCL | Unknown | 32187361 | — | 5: 112840023 . . . 112840023 |
| AXIN1 | tumor_4163639 | 1819100 | p.? | c.879 − | DLBCL | Yes | — | COSU440 | 16: 325030 . . . 325030 |
| | | | | 10347G > C | | | | | |
| AXIN1 | tumor_4194218 | 1819104 | p.? | c.879 − | Burkitt's | Yes | | | |
| | | | | 6062A > T | | | | | |
| AXIN1 | U2940 | 2479246 | p.G69E | c.206G > A | DLBCL | Yes | 24531327 | — | 16: 346820 . . . 346820 |
| AXIN2 | tumor_4163639 | 1819100 | p.? | c.957 − | DLBCL | Yes | — | COSU440 | 17: 65545095 . . . 65545095 |
| | | | | 3538G > A | | | | | |
| AXIN2 | P-0011007-T01- | 2726649 | p.E400D | c.1200G > C | DLBCL | No | 28481359 | — | 17: 65538203 . . . 65538203 |
| GSK3B | tumor_4116738 | 1819095 | p.? | c.88 + | DLBCL | Yes | — | COSU440 | 3: 120055875 . . . 120055876 |
| | | | | 37476dup | | | | | |
| GSK3B | tumor_4163639 | 1819100 | p.? | c.89 − | DLBCL | Yes | — | COSU440 | 3: 120042459 . . . 120042459 |
| | | | | 40220C > T | | | | | |

| | Sample | Sample | Silent | CDS | | MYC- | Pubmed | CGP | |
|---|---|---|---|---|---|---|---|---|---|
| Gene | Name | ID | mutations | Mutation | Histology | break | Id | Study | Genomic GRCh38 |
| CTNNB1 | E13459 | 740580 | p.T40 silent | c.120T > C | DLBCL | No | 11927008 | — | 3: 41224632 . . . 41224632 |
| CTNNB1 | CH-109-T2 | 2462827 | p.G725 silent | c.2175C > T | DLBCL | No | 26647218 | — | 3: 41239171 . . . 41239171 |
| APC | QC2-42-T2 | 2462820 | p.S1110 silent | c.3330A > G | DLBCL | No | 26647218 | — | 5: 112838924 . . . 112838924 |
| AXIN2 | QC2-18-T2 | 2462797 | p.L82 silent | c.246A > G | DLBCL | No | 26647218 | — | 17: 65558375 . . . 65558375 |

Based on genetic knockin mutation of a single Ikaros-binding motif the critical importance of the Myc BENC-C superenhancer region as mechanistic basis for GSK3β-inhibitor activity was demonstrated (FIG. 6). Recent work demonstrated far-reaching effects of aberrant somatic hypermutation targeting superenhancer regions, including MYC, in diffuse large B-cell lymphomas (DLBCL)⁶². Studying MYC BENC superenhancer regions in 93 DLBCL cases and normal germinal center B-cells as a reference, 89 point mutations were found in 49 cases (52.6%, range 1-8 mutations per case) as byproduct of aberrant somatic hypermutation (Table 10). While the Ikaros m1 motif within MYC BENC-C (FIG. 6) was not mutated, these results show that the BENC region Is subject to pervasive hypermutation in DLBCLs and sporadic mutations of the Ikaros motif within the BENC-C region could be a mechanism to confer resistance to GSK3β-inhibition as observed in the CRISPR experiment with an engineered knockin mutation in the Ikaros m1 motif within BENC-C (FIG. 6). In addition, deletion of MYC downstream regions, including BENC, and aberrant overexpression of N-Myc -as observed in T-cell lymphomas⁵⁷, could represent mechanisms that confer resistance to GSK3β-inhibitor treatment.

B-ALL, unmutated CLL and mantle cell lymphomas were derived from pre-germinal center stages of B-cell development that are not subject to somatic hypermutation. In addition, MYC-rearrangements were exceedingly rare in B-ALL, unmutated CLL and MCL. In the present experiments, B-ALL and mantle cell lymphomas were highly sensitive to GSK3β-inhibition in vitro, suggesting that patients with these diseases might benefit from a targeted repurposing effort of GSK3β-inhibitors.

TABLE 10
Aberrant somatic hypermutation of MYC BENC superenhancer regions in DLBCL

| Chr | Start | End | Ref | DLBCL | Func.refGene | Symbol | dbSNP Id | VAE | Sample ID | BENC |
|---|---|---|---|---|---|---|---|---|---|---|
| 8 | 130558974 | 130559436 | | | | | | | | BENC-A |
| 8 | 130560764 | 130560764 | T | A | ncRNA_intronic | CCDC26 | — | 0.59 | 4 | |
| 8 | 130561481 | 130561481 | TT | — | ncRNA_intronic | CCDC26 | — | 0.21 | A68798 | |
| 8 | 130564564 | 130566224 | | | | | | | | BENC-B |
| 8 | 130645692 | 130645692 | AA | AG; GG | ncRNA_intronic | CCDC26 | [symbol] | | | |
| 8 | 130568802 | 130568802 | A | T | ncRNA_intronic | CCDC26 | — | 0.19 | A37785 | |
| 8 | 130569310 | 130569310 | G | T | ncRNA_intronic | CCDC26 | — | 0.52 | CRF-100 | |
| 8 | 130570852 | 130570852 | G | A | ncRNA_intronic | CCDC26 | — | 0.35 | A48305 | |
| 8 | 130570913 | 130570913 | T | A | ncRNA_intronic | CCDC26 | — | 0.33 | A52063 | |
| 8 | 130572094 | 130572094 | C | T | ncRNA_intronic | CCDC26 | — | 0.13 | A51657 | |
| 8 | 130573207 | 130573207 | A | G | ncRNA_intronic | CCDC26 | — | 0.05 | A52053 | |
| 8 | 130574180 | 130574180 | C | T | ncRNA_intronic | CCDC26 | — | 0.35 | A55745 | |
| 8 | 130574318 | 130574318 | C | G | ncRNA_intronic | CCDC26 | — | 0.26 | A55745 | |
| 8 | 130575175 | 130575175 | — | AT | ncRNA_intronic | CCDC26 | — | 0.42 | A48261 | |
| 8 | 130585123 | 130585123 | G | C | ncRNA_intronic | CCDC26 | — | 0.45 | A52055 | |
| 8 | 130585641 | 130585641 | T | — | ncRNA_intronic | CCDC26 | — | 0.25 | A37783 | |
| 8 | 130585641 | 130585641 | T | — | ncRNA_intronic | CCDC26 | — | 0.52 | A48262 | |
| 8 | 130585641 | 130585641 | T | — | ncRNA_intronic | CCDC26 | — | 0.45 | A52057 | |
| 8 | 130585641 | 130585641 | T | — | ncRNA_intronic | CCDC26 | — | 0.2 | CRF-172 | |
| 8 | 130586243 | 130586243 | C | — | ncRNA_intronic | CCDC26 | — | 0.35 | A37783 | |
| 8 | 130587058 | 130587058 | C | T | ncRNA_intronic | CCDC26 | — | 0.14 | A70364 | |
| 8 | 130590151 | 130590151 | — | TTAA | ncRNA_intronic | CCDC26 | — | 0.3 | A68803 | |
| 8 | 130591588 | 130591588 | A | — | ncRNA_intronic | CCDC26 | rs200787134; | 0.24 | A51673 | |
| | | | | | | | rs57500408 | | | |
| 8 | 130591588 | 130591588 | A | — | ncRNA_intronic | CCDC26 | rs200787134; | 0.2 | A68798 | |
| | | | | | | | rs57500408 | | | |
| 8 | 130591588 | 130591588 | A | — | ncRNA_intronic | CCDC26 | rs200787134; | 0.38 | A68803 | |
| | | | | | | | rs57500408 | | | |
| 8 | 130592881 | 130592881 | A | — | ncRNA_intronic | CCDC26 | — | 0.2 | CRF-48 | |
| 8 | 130594181 | 130594694 | | | | | | | | BENC-C |
| 8 | 130597203 | 130597203 | A | T | ncRNA_intronic | CCDC26 | — | 0.35 | A52055 | |
| 8 | 130598146 | 130598146 | A | G | ncRNA_intronic | CCDC26 | — | 0.21 | A37785 | |
| 8 | 130605968 | 130605968 | A | T | ncRNA_intronic | CCDC26 | — | 0.22 | A70373 | BENC-D |
| 8 | 130606652 | 130606652 | A | G | ncRNA_intronic | CCDC26 | — | 0.39 | CRF-174 | |
| 8 | 130608709 | 130608709 | G | T | ncRNA_intronic | CCDC26 | — | 0.23 | CRF-40 | |
| 8 | 130613468 | 130613468 | C | T | ncRNA_intronic | CCDC26 | — | 0.34 | A51614 | |
| 8 | 130616052 | 130616052 | C | T | ncRNA_intronic | CCDC26 | — | 0.26 | CRF-25 | |
| 8 | 130625533 | 130625533 | T | G | ncRNA_intronic | CCDC26 | — | 0.3 | CRF-40 | |
| 8 | 130625885 | 130625885 | T | G | ncRNA_intronic | CCDC26 | — | 0.24 | A68798 | |
| 8 | 130627645 | 130627645 | T | C | ncRNA_intronic | CCDC26 | — | 0.47 | A48261 | |
| 8 | 130627791 | 130627791 | C | T | ncRNA_intronic | CCDC26 | — | 0.29 | CRF-168 | |
| 8 | 130630893 | 130630893 | G | C | ncRNA_intronic | CCDC26 | — | 0.2 | A37905 | |
| 8 | 130632321 | 130632321 | — | A | ncRNA_intronic | CCDC26 | — | 0.18 | A52060 | |
| 8 | 130635486 | 130635486 | T | — | ncRNA_intronic | CCDC26 | — | 0.15 | A52057 | |
| 8 | 130636555 | 130636555 | T | G | ncRNA_intronic | CCDC26 | — | 0.51 | CRF-46 | |
| 8 | 130637810 | 130637810 | T | — | ncRNA_intronic | CCDC26 | Mn9393703 | 0.64 | A70373 | |
| 8 | 130637861 | 130637861 | C | T | ncRNA_intronic | CCDC26 | — | 0.18 | A52061 | |
| 8 | 130639181 | 130639181 | C | T | ncRNA_intronic | CCDC26 | — | 0.19 | A52060 | |
| 8 | 130639381 | 130639381 | A | C | ncRNA_intronic | CCDC26 | — | 0.17 | A68798 | |
| 8 | 130641180 | 130641180 | C | T | ncRNA_intronic | CCDC26 | — | 0.46 | A48305 | |
| 8 | 130641252 | 130641252 | C | T | ncRNA_intronic | CCDC26 | — | 0.23 | A52053 | |
| 8 | 130641654 | 130641654 | A | C | ncRNA_intronic | CCDC26 | — | 0.39 | CRF-178 | |
| 8 | 130643843 | 130643843 | A | T | ncRNA_intronic | CCDC26 | — | 0.71 | CRF-174 | |
| 8 | 130645521 | 130645521 | C | A | ncRNA_intronic | CCDC26 | — | 0.1 | A48306 | |
| 8 | 130647649 | 130647649 | A | C | ncRNA_intronic | CCDC26 | — | 0.28 | CRF-25 | |
| 8 | 130647830 | 130647830 | A | — | ncRNA_intronic | CCDC26 | — | 0.3 | A51614 | |
| 8 | 130647830 | 130647830 | A | — | ncRNA_intronic | CCDC26 | — | 0.23 | A68798 | |
| 8 | 130648267 | 130648647 | | | | | | | | BENC-F |
| 8 | 130650005 | 130650005 | T | A | ncRNA_intronic | CCDC26 | — | 0.3 | A55509 | |
| 8 | 130650571 | 130650571 | G | A | ncRNA_intronic | CCDC26 | — | 0.31 | A52063 | |
| 8 | 130652006 | 130652006 | C | G | ncRNA_intronic | CCDC26 | — | 0.46 | A70373 | |
| 8 | 130652187 | 130652187 | A | — | ncRNA_intronic | CCDC26 | — | 0.36 | A68798 | |
| 8 | 130653052 | 130653052 | A | T | ncRNA_intronic | CCDC26 | — | 0.14 | CRF-94 | |
| 8 | 130654026 | 130654026 | T | — | ncRNA_intronic | CCDC26 | rs35334077; | 0.32 | A52063 | |
| | | | | | | | rs398009897 | | | |
| 8 | 130658740 | 130658740 | C | G | ncRNA_intronic | CCDC26 | — | 0.28 | CRF-171 | |
| 8 | 130660826 | 130660826 | G | C | ncRNA_intronic | CCDC26 | — | 0.27 | A55511 | |
| 8 | 130663274 | 130663274 | A | C | ncRNA_intronic | CCDC26 | — | 0.28 | A68798 | |
| 8 | 130664590 | 130664590 | T | — | ncRNA_intronic | CCDC26 | — | 0.2 | A55511 | |
| 8 | 130664590 | 130664590 | T | — | ncRNA_intronic | CCDC26 | — | 0.45 | A55514 | |
| 8 | 130665383 | 130665383 | — | A | ncRNA_intronic | CCDC26 | — | 0.35 | A70378 | |
| 8 | 130669521 | 130669521 | T | G | ncRNA_intronic | CCDC26 | — | 0.05 | A52056 | |
| 8 | 130669720 | 130669720 | G | T | ncRNA_intronic | CCDC26 | — | 0.3 | A52063 | |
| 8 | 130673406 | 130673406 | G | A | ncRNA_intronic | CCDC26 | — | 0.32 | CRF-168 | |
| 8 | 130673745 | 130673745 | A | C | ncRNA_intronic | CCDC26 | — | 0.15 | CRF-48 | |
| 8 | 130674815 | 130674815 | A | — | ncRNA_intronic | CCDC26 | — | 0.22 | A52061 | |
| 8 | 130675704 | 130675704 | A | — | ncRNA_intronic | CCDC26 | rs11345336; | 0.15 | A51657 | |
| | | | | | | | rs796600759 | | | |
| 8 | 130676216 | 130676216 | T | C | ncRNA_intronic | CCDC26 | — | 0.38 | A48305 | |
| 8 | 130679002 | 130679002 | G | A | ncRNA_intronic | CCDC26 | — | 0.14 | A68784 | BENC-G |
| 8 | 130679720 | 130679720 | A | C | ncRNA_intronic | CCDC26 | — | 0.14 | A52061 | |
| 8 | 130681297 | 130681297 | T | C | ncRNA_intronic | CCDC26 | — | 0.2 | A51310 | |
| 8 | 130681359 | 130681359 | A | T | ncRNA_intronic | CCDC26 | — | 0.3 | A70382 | |
| 8 | 130685014 | 130685014 | C | A | ncRNA_intronic | CCDC26 | — | 0.3 | A68788 | |
| 8 | 130685075 | 130685281 | | | | | | | | BENC-H |
| 8 | 130689547 | 130689547 | C | T | ncRNA_intronic | CCDC26 | — | 0.21 | 1 | |
| 8 | 130691134 | 130691134 | A | G | ncRNA_intronic | CCDC26 | — | 0.31 | 5 | |
| 8 | 130696294 | 130696294 | A | — | intergenic | CCDC26; GSDMC | — | 0.27 | A55506 | |
| 8 | 130698799 | 130698799 | T | G | intergenic | CCDC26; GSDMC | — | 0.34 | A51673 | |
| 8 | 130699522 | 130699522 | C | T | intergenic | CCDC26; GSDMC | — | 0.11 | A70385 | |
| 8 | 130702011 | 130702011 | — | A | intergenic | CCDC26; GSDMC | — | 0.13 | CRF-94 | |
| 8 | 130705012 | 130705415 | | | | | | | | BENC-1 |
| 8 | 130707443 | 130707443 | A | — | intergenic | CCDC26; GSDMC | — | 0.21 | A68798 | |
| 8 | 130710562 | 130710562 | G | A | intergenic | CCDC26; GSDMC | — | 0.23 | A68799 | |
| 8 | 130713186 | 130713186 | A | — | intergenic | CCDC26; GSDMC | — | 0.27 | A70364 | |
| 8 | 130714070 | 130714070 | A | G | intergenic | CCDC26; GSDMC | — | 0.08 | A48306 | |
| 8 | 130718646 | 130718646 | A | C | intergenic | CCDC26; GSDMC | — | 0.31 | A52061 | |
| 8 | 130721419 | 130721419 | T | — | intergenic | CCDC26; GSDMC | Mn9394212; | 0.31 | A68794 | |
| | | | | | | | rs59912996; rs | | | |
| 8 | 130721904 | 130721904 | A | C | intergenic | CCDC26; GSDMC | — | 0.22 | A70385 | |
| 8 | 130727836 | 130727836 | A | T | intergenic | CCDC26; GSDMC | — | 0.13 | A48306 | |
| 8 | 130730745 | 130730745 | G | T | intergenic | CCDC26; GSDMC | — | 0.13 | CRF-170 | |

[symbol]

indicates data missing or illegible when filed

For the past 60 years, glucocorticoids have been central empirical components of nearly all treatment regimen for B-lymphoid malignancies⁶³. Likewise, L-asparaginase and methotrexate⁶⁴⁻⁶⁵ have highly selective effects on B-lymphoid malignancies with very limited toxicity in myeloid and epithelial cells⁶³⁻⁶⁵. More recently, antibody-mediated killing (e.g., rituximab) and inhibition of B-cell receptor signaling (e.g. ibrutinib⁶⁶⁻⁶⁷) have built on the concept of targeted elimination of B-lymphoid cells, with B-cell depletion and lymphopenia as acceptable side-effect. While these treatments have revolutionized the treatment of B-cell malignancies, the frequent development of resistance (e.g. to glucocorticoids in B-ALL and ibrutinib in B-cell lymphomas) has limited their overall success. Given their B-cell-selective activity, the future repurposing efforts of GSK3β-inhibitors will focus on opportunities to overcome resistance to glucocorticoids in B-ALL and ibrutinib in B-cell lymphomas.

### Example 19. CTNNB1-Deletion in Human Hematopoietic Progenitor Cells Improves Lymphoid Lineage Reconstitution

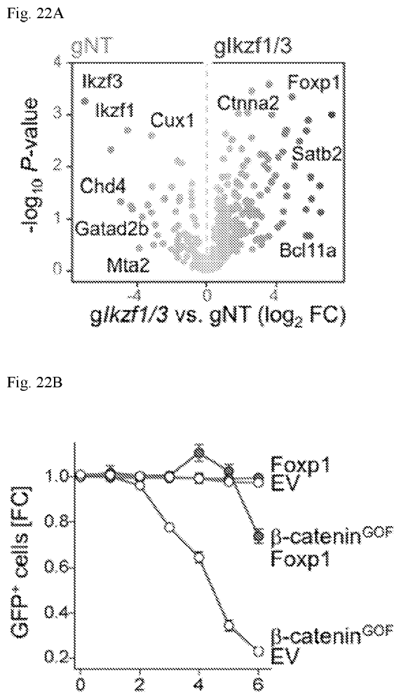

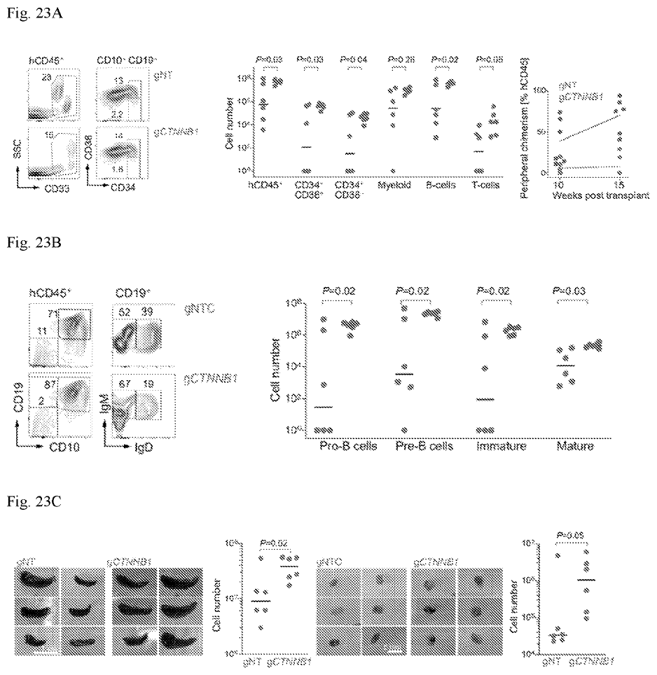

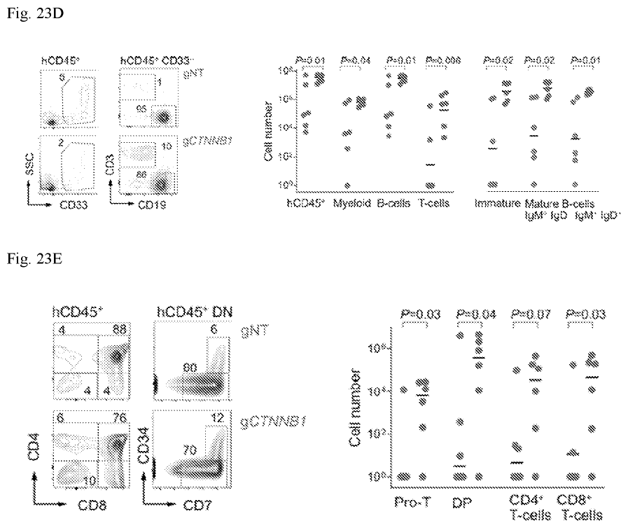

Lymphopenia can be caused by decreased lymphocyte production as a common feature of immunosenescence in elderly individuals and is associated with substantially increased mortality³³. Other causes of defective lymphopoiesis include side effects of drug-treatment, bone marrow transplantation, viral infections and immunodeficiencies. In elderly individuals, myeloid skewing of hematopoietic stem cells results in a relative increase of myelopoiesis at the expense of lymphocyte production³⁴⁻³⁵. Given that β-catenin engages lymphoid-specific IKZF-factors to repress MYC and restrict lymphoid cell proliferation, it was reasoned that genetic deletion of CTNNB1 in human hematopoietic progenitor cells could selectively benefit lymphoid development, which could be useful under lymphopenic conditions, immunosenescence and myeloid skewing. To this end, genetic deletion of CTNNB1 in human CD34⁺ cord blood-derived hematopoietic progenitor cells was engineered using Cas9 RNPs with guide-RNAs directed against CTNNB1 (gCTNNB1) or non-targeting controls (gNT) and injected 100,000 progenitor cells into unconditioned NSGW41 mice for multi-lineage reconstitution of human hematopoiesis (FIG. 23). Compared to controls, CTNNB1-deleted progenitor cells showed accelerated engraftment and increased human chimerism in the peripheral blood of NSGW41 recipient mice 10 and 15 weeks after transplantation. The relative proportions of progenitor, myeloid and lymphoid populations were similar in both groups with a slight shift towards B-lymphopoiesis at the expense of the myeloid compartment. Comparing absolute cell numbers, β-catenin-deficient progenitors gave rise to markedly increased human B-cell and T-cell production, while myeloid populations were largely unchanged. The spleens of NSGW41 mice transplanted with β-catenin-deficient progenitors were significantly larger and colonized by human B- and T-lymphoid cells at greater numbers. Upon injection of human hematopoietic progenitor cells, NSGW41 mice developed a functional thymus, which contained significantly more human thymocytes in the β-catenin-deficient group (FIG. 23). After 15 weeks, only few mature human T-cells had colonized the spleen under control conditions, which was dramatically accelerated when CTNNB1-deleted progenitor cells were injected. CD34⁺ cord blood hematopoietic progenitor cells successfully engrafted in NSGW41 mice to give rise to human multilineage reconstitution under control conditions. However, in the absence of β-catenin, the production of human B- and T-lymphoid cells was increased on average by 132-fold (B-cells) and 6,520-fold (T-cells), while the number of myeloid cells remained largely unchanged. These findings suggest that genetic engineering of CTNNB1-deletion does not adversely impact human hematopoiesis and instead relieves lymphoid-intrinsic repression of B- and T-lymphopoiesis by β-catenin:IKZF complexes.

### Example 20. Engineered Deletion of CTNNB1 Improves Lymphopoiesis from MDS Progenitor Cells

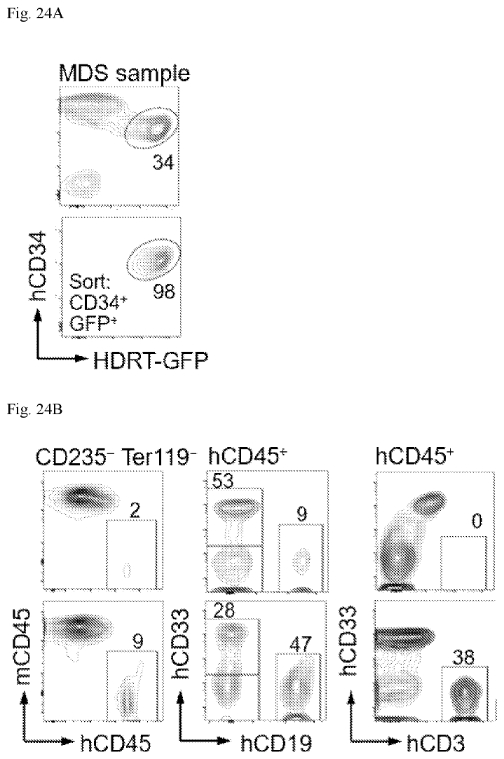

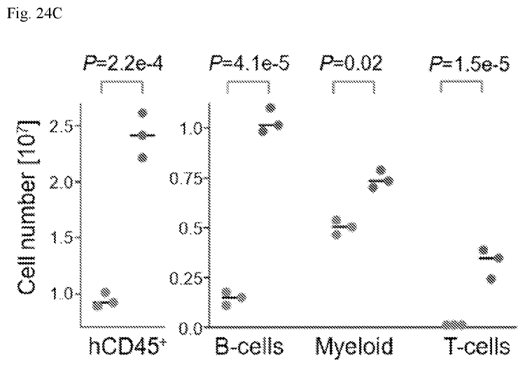

Feasibility of genetic engineering and subsequent hematopoietic reconstitution was established based on CD34⁺ progenitor cells enriched from bone marrow samples from three patients with myelodysplastic syndromes (MDS). Engineered deletion of CTNNB1 with a GFP-knockin allele as an ssDNA repair template in MDS-derived CD34⁺ bone marrow progenitors and subsequent flow sorting resulted in a 98% CD34+ GFP+ population, which were injected into MISTRG mice for human multilineage reconstitution (FIG. 24).

Non-targeted and targeted CD34⁺ GFP⁺ cells were stimulated in the presence of human cytokines and colony forming assays were performed. Large colonies were analyzed by Western blot after 12 days. Small molecule GSK3β-inhibition (LY2090314) induced accumulation of β-catenin in all 7 non-targeted control colonies. Among 15 targeted colonies, 13 lacked the ability to express β-catenin, while 2 colonies exhibited faint expression, consistent with possible heterozygous deletion. These results show that the present approach using ssDNA repair templates for engineered deletion of CTNNB1 and flow sorting of GFP-knockin allele expressing cells achieves complete deletion in >80% of targeted bone marrow progenitor cells.

After HDRT-mediated deletion of CTNNB1, 100,000 CD34⁺ GFP⁺ progenitor cells from bone marrow samples of three MDS-patients (purity 96-98%) were transplanted into MISTRG mice. After 16 weeks, mice were sacrificed, and human hematopoietic cells were analyzed. Compared to cord blood, multilineage reconstitution of human hematopoiesis was significantly weaker from the MDS samples. Compared to non-targeted progenitors, CTNNB1^{-/-} progenitors gave rise to substantially increased B-lymphopoiesis and slightly increased myelopoiesis. Strikingly, progenitor cells from MDS bone marrows were not able to give rise to thymopoiesis and T-cell production, unless CTNNB1 was deleted (FIG. 23). In the presence of CTNNB1, MISTRG mice did not form a functional thymus and did not develop any human T-cells.

In addition to multilineage reconstitution analysis, additional readouts include clonality studies of B- and T-cell development based on Ig- and TCR-repertoire sequencing. For 3 MDS and 3 CHIP matched pairs (non-targeted vs CTNNB1-deleted), single-cell RNA-seq analyses is performed to determine how hematopoietic populations shift in their size and whether clonal hematopoietic populations that likely exist in MDS and CHIP-samples are selectively impacted by CTNNB1-deletion. For functional characterization of B- and T-cells that develop in the absence of CTNNB1, BCR- and TCR-responsiveness and the ability of myeloid cell populations to respond to LPS and other TLR-ligands in vitro is tested.

It is anticipated that CTNNB1-deletion represents a useful strategy to improve cytopenic conditions in the context of clonal hematopoiesis and MDS: while β-catenin is dispensable for normal hematopoiesis, genetic mouse models previously showed an essential role of β-catenin in leukemia-initiation towards overt AML and CML. Hence, CTNNB1-deletion may improve hematopoietic output and at the same time shield against malignant progression and leukemia-initiation from CHIP and MDS.

### REFERENCES

1. Logan C. Y. and Nusse, R. (2004). The Wnt signaling pathway in development and disease. Annu. Rev. Cell Dev. Biol. 20, 781-810.
2. Korinek V. Barker N., Morin P. J., van Wichen D., de Weger R., Kinzler K. W., Vogelstein B., Clevers H. (1997). Constitutive transcriptional activation by a beta-catenin-Tcf complex in APC−/− colon carcinoma. Science 21, 1784-7.
3. Roose J., Huls G., van Beest M., Moerer P., van der Horn K., Goldschmeding R., Logtenberg T., Clevers H. (1999). Synergy between tumor suppressor APC and the beta-catenin-Tcf4 target Tcfl. Science 285, 1923-6.
4. Park J. I., Venteicher A. S., Hong J. Y., Choi J., Jun S., Shkreli M., Chang W., Meng Z., Cheung P., Ji H., McLaughlin M., Veenstra T. D., Nusse R., McCrea P. D. & Artandi S. E. (2009). Telomerase modulates Wnt signalling by association with target gene chromatin. Nature 460, 66-72.
5. Sansom O. J. Meniel V. S., Muncan V., Phesse T. J., Wilkins J. A., Reed K. R., Vass J K., Athineos D., Clevers H., Clarke A. R. (2007). Myc deletion rescues Apc deficiency in the small intestine. Nature 446 676-9.
6. He T. C., Sparks A. B., Rago C., Hermeking H., Zawel L., da Costa L. T., Morin P. J., Vogelstein B., Kinzler K. W. (1998). Identification of c-MYC as a target of the APC pathway. Science 281, 1509-12.
7. Behrens J., Jerchow B. A., Wurtele M., Grimm J., Asbrand C., Wirtz R., K0 hl M., Wedlich D., Birchmeier W. (1998). Functional interaction of an Axin homolog, conductin, with beta-catenin, APC, and GSK3beta. Science 280, 596-9.
8. Liu C., Li Y., Semenov M., Han C., Baeg G. H., Tan Y., Zhang Z., Lin X., He X. (2002). Control of beta-catenin phosphorylation/degradation by a dual-kinase mechanism. Cell 108, 837-847.
9. Zeng X., Huang H., Tamai K., Zhang X., Harada Y., Yokota C., Almeida K., Wang J., Doble B., Woodgett J., Wynshaw-Boris A., Hsieh J., He X. (2008). Initiation of Wnt signaling control of Wnt coreceptor Lrp6 phosphorylation/activation via frizzled, dishevelled and axin functions. Development 135, 367-75.
10. Cobas M., Wilson A., Ernst B., Mancini S. J. C., MacDonald H. R., Kemler R., Radtke F. (2004). Beta-catenin is dispensable for hematopoiesis and lymphopoiesis. J. Exp. Med. 199, 221-9.
11. Koch U., Wilson A., Cobas M., Kemler R., Macdonald H. R., Radtke F. (2008). Simultaneous loss of β- and γ-catenin does not perturb hematopoiesis or lymphopoiesis. Blood 111, 160-164.
12. Zhao X., Shao P., Gai K., Li F, Shan Q., Xue H. H. (2020). β-catenin and y-catenin are dispensable for T lymphocytes and AML leukemic stem cells. Elife 9, e55360.
13. Luis T. C., Naber B. A., Roozen P. P., Brugman M. H., de Haas E. F., Ghazvini M., Fibbe W. E., van Dongen J. J., Fodde R., Staal F. J. (2011) Canonical Wnt signaling regulates hematopoiesis in a dosage-dependent fashion. Cell Stem Cell 9, 345-56.
14. Xie H., Huang Z., Sadim M. S., Sun Z. (2005). Stabilized beta-catenin extends thymocyte survival by up-regulating Bcl-xL. Journal of Immunology 175, 7981-7988.
15. Ding Y., Shen S., Lino A. C., Curotto de Lafaille M. A., Lafaille J. J. (2008) Beta-catenin stabilization extends regulatory T cell survival and induces anergy in nonregulatory T cells. Nature Medicine 14, 162-169.
16. Gattinoni L., Zhong X. S., Palmer D. C., Ji Y., Hinrichs C. S., Yu Z., Wrzesinski C., Boni A., Cassard L, Garvin L. M., Paulos C. M., Muranski P., Restifo N. P. (2009) Wnt signaling arrests effector T cell differentiation and generates CD8+ memory stem cells Nature Medicine 15, 808-813.
17. Kirstetter P., Anderson K., Porse B. T., Jacobsen S. E. W. & Nerlov C. (2006). Activation of the canonical Wnt pathway leads to loss of hematopoietic stem cell repopulation and multilineage differentiation block. Nat. Immunol. 7, 1048-56.
18. Scheller M., Huelsken J., Rosenbauer F., Taketo M. M., Birchmeier W., Tenen D. G., Leutz A. (2006). Hematopoietic stem cell and multilineage defects generated by constitutive β-catenin activation Nat. Immunol. 7, 1037-1047.
19. Guo Z., Dose M., Kovalovsky D., Chang R., O'Neil J., Look A. T., von Boehmer H., Khazaie K., Gounari F. (2007). β-catenin stabilization stalls the transition from double-positive to single-positive stage and predisposes thymocytes to malignant transformation. Blood 109, 5463-5472.
20. Morin P. J., Sparks A. B., Korinek V., Barker N., Clevers H., Vogelstein B., Kinzler K. W. (1997). Activation of beta-catenin-Tcf signaling in colon cancer by mutations in β-catenin or APC. Science 275, 1787-90.
21. Rubinfeld B., Sparks A. B., Korinek V., Barker N., Clevers H., Vogelstein B., Kinzler K. W. (1997). Stabilization of beta-catenin by genetic defects in melanoma cell lines. Science 275, 1790-2.
22. Wang Y. Krivtsov A. V., Sinha A. U., North T. E., Goessling W., Feng Z., Zon L. I., Armstrong S. A. (2010). The Wnt/beta-catenin pathway is required for the development of leukemia stem cells in AML. Science 327, 1650-3.
23. Yeung J., Esposito M. T., Gandillet A., Zeisig B. B., Griessinger E., Bonnet D., So C. W. (2010) β-Catenin mediates the establishment and drug resistance of MLL leukemic stem cells. Cancer Cell 18, 606-618.
24. Zhao C., Blum J., Chen A., Kwon H. Y., Jung S. H., Cook J. M., Lagoo A., Reya T. (2007).

Loss of beta-catenin impairs the renewal of normal and CML stem cells in vivo. Cancer Cell 12, 528-41.
25. Dose M., Emmanuel A. O., Chaumeil J., Zhang J., Sun T., Germar K., Aghajani K., Davis E. M., Keerthivasan S., Bredemeyer A. L., Sleckman B. P., Skok J. A., Le Beau M. M., Georgopoulos K., Gounari F. (2014). β-Catenin induces T-cell transformation by promoting genomic instability. Proc Natl Acad Sci USA. 111, 391-6.
26. Gekas C., D′Altri T., Aligue R., Gonzilez J., Espinosa L., Bigas A. (2016). β-Catenin is required for T-cell leukemia initiation and MYC transcription downstream of Notch1. Leukemia., 10, 2002-2010.
27. COSMIC: Catalogue of somatic mutations in cancer https://cancer.sanger.ac.uk/cosmic/gene/analysis? ln=CTNNB1
28. de Roo J. J., Breukel C., Chhatta A. R., Linssen M. M., Vloemans S. A., Salvatori D., Mikkers H. M. M, Verbeek S. J., Staal F. J. T. (2017). Axin2-mTurquoise2: A novel reporter mouse model for the detection of canonical Wnt signalling. Genesis 55, e23068.
29. DepMap: The Cancer Dependency Map Project at Broad Institute. https://depmap.org/portal/gene/CTNNB1?tab=characterization
30. Harada N., Tamai Y., Ishikawa T., Sauer B., Takaku K., Oshima M., Taketo M. M. (1999). Intestinal polyposis in mice with a dominant stable mutation of the beta-catenin gene. EMBO J. 18, 5931-5942.
31. Georgopoulos K. (2017). The making of a lymphocyte: the choice among disparate cell fates and the IKAROS enigma. Genes Dev. 31, 439-450.
32. Hu Y., Zhang Z., Kashiwagi M., Yoshida T., Joshi I., Jena N., Somasundaram R., Emmanuel A. O., Sigvardsson M., Fitamant J., El-Bardeesy N., Gounari F., Van Etten R. A. (2016). Superenhancer reprogramming drives a B-cell-epithelial transition and high-risk leukemia. Genes Dev. 30, 1971-90.
33. Zhang J., Jackson A. F., Naito T., Dose M., Seavitt J., Liu F., Heller E. J., Kashiwagi M., Yoshida T., Gounari F., Petrie H. T., Georgopoulos K. (2011). Harnessing of the nucleosome-remodeling-deacetylase complex controls lymphocyte development and prevents leukemogenesis. Nat. Immunol. 13, 86-94.
34. Arends T., Dege C., Bortnick A., Danhorn T., Knapp J. R., Jia H., Harmacek L., Fleenor C. J., Straign D., Walton K., Leach S. M., Feeney, A. J., Murrec C., O'Connor B. P., and Hagman J. R. (2019). CHD4 is essential for transcriptional repression and lineage progression in B lymphopoiesis. Proc. Natl. Acad. Sci. USA 116, 10927-10936.
35. Lu X., Chu C. S., Fang T., Rayon-Estrada V., Fang F., Patke A., Qian Y., Clarke S. H., Melnick A. M., Zhang Y., Papavasiliou F. N., Roeder R. G. (2019). MTA2/NuRD Regulates B Cell Development and Cooperates with OCA-B in Controlling the Pre-B to Immature B Cell Transition. Cell Rep. 28 472-485.e5.
36. Loughran S. J., Comoglio F., Hamey F. K., Giustacchini A., Errami Y., Earp E., Gottgens B., Jacobsen S. E. W., Mead A. J., Hendrich B., Green A. R. (2017). Mbd3/NuRD controls lymphoid cell fate and inhibits tumorigenesis by repressing a B cell transcriptional program. J Exp Med. 214, 3085-3104.
37. Bauer A., Huber O., Kemler R. (1998). Pontin52, an interaction partner of beta-catenin, binds to the TATA box binding protein. Proc. Natl. Acad. Sci. USA. 95, 14787-92.
38. Wood M. A., McMahon S. B., Cole M. D. (2000). An ATPase/helicase complex is an essential cofactor for oncogenic transformation by c-Myc. Mol Cell. 5, 321-30.
39. Mullighan C. G., Su X., Zhang J., Radtke I., Phillips L. A. A., Miller C. B., Ma J., Liu W., Cheng C., Schulman B. A., Harvey R. C., Chen I., Clifford R. J., Carroll W. L., Reaman G., Bowman W. P., Devidas M., Gerhard D. S., Yang W., Relling M. V., Shurtleff S. A., Campana D., Borowitz M. J., Pui C., Smith M., Hunger S. P., Willman C. L., Downing J. R., Children's Oncology Group. (2009). Deletion of IKZF1 and prognosis in acute lymphoblastic leukemia. N. Engl. J. Med. 360, 470-480.
40. Lazarian G.,Yin S., Hacken E. T., Sewastianik T., Uduman M, Font-Tello A., Gohil S. H., Li S., Kim E, Joyal H, Billington L., Witten E., Zheng M., Huang T., Severgnini M., Lefebvre V., Rassenti L. Z., Gutierrez C., Georgopoulos K., Ott C. J., Wang L., Kipps T. J., Burger J. A., Livak K. J, Neuberg D. S., Baran-Marszak F., Cymbalista F., Carrasco R. D., Wu C. J. (2021). A hotspot mutation in transcription factor IKZF3 drives B cell neoplasia via transcriptional dysregulation. Cancer Cell. 39, 380-393.
41. Krönke J., Udeshi N. D., Narla A., Grauman P., Hurst S. N., McConkey M., Svinkina T., Heckl D., Comer E., Li X., Ciarlo C., Hartman E., Munshi N., Schenone M., Schreiber S. L., Carr S. A., Ebert B. L. (2014). Lenalidomide causes selective degradation of IKZF1 and IKZF3 in multiple myeloma cells. Science 343, 301-305.
42. Lu G., Middleton R. E., Sun H., Naniong M., Ott C. J., Mitsiades C. S., Wong K., Bradner J. E., Kaelin W. G. (2014). The myeloma drug lenalidomide promotes the cereblon-dependent destruction of Ikaros proteins. Science 343, 305-309.
43. Barnell E. K., Skidmore Z. L., Newcomer K. F., Chavez M., Campbell K. M., Cotto K. C., Spies N. C., Ruzinova M. B., Wang T., Abro B., Kreisel F., Parikh B. A., Duncavage E. J., Frater J. L., Lee Y. S., Hassan A., King J. A., Kohnen D. R., Fiala M. A., Welch J. S., Uy G. L., Vij K., Vij R., Griffith M., Griffith O. L., Wartman L. D. (2023) Distinct clonal identities of B-ALLs arising after lenolidomide therapy for multiple myeloma. Blood Adv. 7, 236-245.
44. Furstenau M., Fink A. M., Schilhabel A., Weiss J., Robrecht S., Eckert R., de la Serna J., Crespo M., Coscia M., Vitale C., Bottcher S., Weppner G., Ritgen M., Stilgenbauer S., Tausch E., Fischer K., Hallek M., Eichhorst B., Brüggemann M., Herling C. D. (2021) B-cell acute lymphoblastic leukemia in patients with chronic lymphocytic leukemia treated with lenalidomide. Blood 137, 2267-2271.
45. Bahr C., von Paleske L., Uslu V. V., Remeseiro S., Takayama N., Ng S. W., Murison A., Langenfeld K., Petretich M., Scognamiglio R., Zeisberger P., Benk A. S., Amit I., Zandstra P. W., Lupien M., Dick J. E., Trumpp A. & Spitz F. (2018). A Myc enhancer cluster regulates normal and leukaemic haematopoietic stem cell hierarchies. Nature 553, 515-520.
46. DepMap: The Cancer Dependency Map Project at Broad Institute. https://depmap.org/portal/compound/LY2090314?tab=dependency&dependency=Repurposing_secondary_AUC
47. Gray J. E., Infante J. R., Brail L. H., Simon G. R., Cooksey J. F., Jones S. F., Farrington D. L., Yeo A., Jackson K. A., Chow K. H., Zamek-Gliszczynski M. J., Burris H. A. (2015) A first-in-human phase I dose-escalation, pharmacokinetic and pharmacodynamic evaluation of intravenous LY2090314, a glycogen synthase kinase 3 inhibitor, administered in combination with pemetrexed and carboplatin. Invest. New Drugs 33, 1187-1196.
48. Georgievska B., Sandin J., Doherty J., Mörtberg A., Neelissen J., Andersson A., Gruber S., Nilsson Y., Schott P., Arvidsson P. I., Hellberg S., Osswald G., Berg S., Fälting J., Bhat R. V. (2013). AZD1080, a novel GSK3 inhibitor, rescues synaptic plasticity deficits in rodent brain and exhibits peripheral target engagement in humans. J Neurochem. 125, 446-56.
49. McLean W. J., Hinton A. S., Herby J. T. J., Salt A. N., Hartsock J. J., Wilson S., Lucchino D. L., Lenarz T., Warnecke A., Prenzler N., Schmitt H., King S., Jackson L. E., Rosenbloom J., Atiee G., Bear M., Runge C. L., Gifford R. H., Rauch S. D., Lee D. J., Langer R., Karp J. M., Loose C., LeBel C. (2021). Improved Speech Intelligibility in Subjects With Stable Sensorineural Hearing Loss Following Intratympanic Dosing of FX-322 in a Phase lb Study. Otol Neurotol. 42, e849-e857.
50. Horrigan J., Gomes T. B., Snape M., Nikolenko N., McMorn A., Evans S., Yaroshinsky A., Della Pasqua O., Oosterholt S., Lochmuller H. (2020) A Phase 2 Study of AMO-02 (Tideglusib) in Congenital and Childhood-Onset Myotonic Dystrophy Type 1 (DM1). Pediatr Neurol 112, 84-93.
51. Tolosa E., Litvan I., Höglinger G. U., Burn D., Lees A., Andrés M. V., Gómez-Carrillo B., León T., Del Ser T. (2014). TAUROS Investigators. A phase 2 trial of the GSK-3 inhibitor tideglusib in progressive supranuclear palsy. Mov. Disord. 4, 470-8.
52. Lovestone S., Boada M., Dubois B., Hull M., Rinne J. O., Huppertz H. J., Calero M., Andres M. V., Gómez-Carrillo B., León T., del Ser T. (2015). ARGO investigators. A phase II trial of tideglusib in Alzheimer's disease. J Alzheimers Dis. 45, 75-88.
53. Shaw G., Cavalcante L., Giles F. J., Taylor A. (2022). Elraglusib (9-ING-41), a selective small-molecule inhibitor of glycogen synthase kinase-3 beta, reduces expression of immune checkpoint molecules PD-1, TIGIT and LAG-3 and enhances CD8+ T cell cytolytic killing of melanoma cells. J Hematol. Oncol. 15, 134.
54. Choi J. H., Zhong X., McAlpine W., Liao T. C., Zhang D., Fang B., Russell J., Ludwig S., Nair-Gill E., Zhang Z., Wang K. W., Misawa T., Zhan X., Choi M., Wang T., Li X., Tang M., Sun Q., Yu L., Murray A. R., Moresco E. M. Y., Beutler B. (2019). LMBRIL regulates lymphopoiesis through Wnt/β-catenin signaling. Science 364, eaau0812.
55. Georgopoulos K., Bigby M., Wang J. H., Molnar A., Wu P., Winandy S., Sharpe A. (1994). The Ikaros gene is required for the development of all lymphoid lineages. Cell 79, 143-56.
56. Witkowski M. T., Cimmino L., Hu Y., Trimarchi T., Tagoh H., McKenzie M. D., Best S. A., Tuohey L., Willson T. A., Nutt S. L., Busslinger M., Aifantis I., Smyth G. K., Dickins R. A. (2015). Activated Notch counteracts Ikaros tumor suppression in mouse and human T-cell acute lymphoblastic leukemia. Leukemia. 6, 1301-11.
57. Kaveri D., Kastner P., Dembélé D., Nerlov C., Chan S., Kirstetter P. (2013). β-Catenin activation synergizes with Pten loss and Myc overexpression in Notch-independent T-ALL. Blood 122, 694-704.
58. Reya T, O'Riordan M, Okamura R, Devaney E, Willert K, Nusse R, Grosschedl R. (2000). Wnt signaling regulates B lymphocyte proliferation through a LEF-1 dependent mechanism. Immunity 13, 15-24.
59. Dalla-Favera, R., Bregni, M., Erikson, J., Patterson, D., Gallo, R. C., Croce, C. M. (1982). Human c-myc onc gene is located on the region of chromosome 8 that is translocated in Burkitt lymphoma cells. Proc. Natl. Acad. Sci. USA 79, 7824-7827.
60. Taub, R., Kirsch, I., Morton, C., Lenoir, G., Swan, D., Tronick, S., Aaronson, S., Leder, P. (1982). Translocation of the c-myc gene into the immunoglobulin heavy chain locus in human Burkitt lymphoma and murine plasmacytoma cells. Proc. Natl. Acad. Sci. USA 79, 7837-7841.
61. Choi P. S., Li Y, Felsher D. W. (2014). Addiction to multiple oncogenes can be exploited to prevent the emergence of therapeutic resistance. Proc Natl Acad Sci USA. 111, E3316-24.
62. Bal E., Kumar R., Hadigol M., Holmes A. B., Hilton L. K., Loh J. W., Dreval K., Wong J. C. H., Vlasevska S., Corinaldesi C., Soni R. K., Basso K., Morin R. D., Khiabanian H., Pasqualucci L., Dalla-Favera R. (2022). Super-enhancer hypermutation alters oncogene expression in B cell lymphoma. Nature 607, 808-815.
63. Pearson O H, Eliel L P, et al. (1949). Adrenocorticotropic hormone- and cortisone-induced regression of lymphoid tumors in man; a preliminary report. Cancer 2, 943-945.
64. Schrek R, Dolowy W C, Ammeraal RN (1967). L-asparaginase: toxicity to normal and leukemic human lymphocytes. Science 155, 329-30.
65. Holland J F (1967). Recent advances in the treatment of acute lymphoblastic leukemia and Burkitt's tumor. Cancer Res 27, 2633-5.
66. Wang M L, Rule S, Martin P, Goy A, Auer R, Kahl B S, Jurczak W, Advani R H, Romaguera J E, Williams M E, Barrientos J C, Chmielowska E, Radford J, Stilgenbauer S, Dreyling M, Jedrzejczak W W, Johnson P, Spurgeon S E, Li L, Zhang L, Newberry K, Ou Z, Cheng N, Fang B, McGreivy J, Clow F, Buggy J J, Chang B Y, Beaupre D M, Kunkel L A, Blum K A (2013). Targeting BTK with ibrutinib in relapsed or refractory mantle-cell lymphoma. N Engl J Med 369, 507-16.
67. Byrd J C, Furman R R, Coutre S E, Flinn I W, Burger J A, Blum K A, Grant B, Sharman J P, Coleman M, Wierda W G, Jones J A, Zhao W, Heerema N A, Johnson A J, Sukbuntherng J, Chang B Y, Clow F, Hedrick E, Buggy J J, James D F, O'Brien S (2013). Targeting BTK with ibrutinib in relapsed chronic lymphocytic leukemia. N Engl J Med 369, 32-42.
68. Patro R., Duggal G., Love M. t, Irizarty R. A. & Kingsford C. (2017). Salmon provides fast and bias-aware quantification of transcript expression. Nature Methods 14, 417-419.
69. Dobin, C. A., Davis C. A., Schlesinger F., Drenkow J., Zaleski C., Jha S., Batut P., Chaisson M., Gingeras T. R. (2013). STAR: ultrafast universal RNA-seq aligner. Bioinformatics 29, 15-21.
70. R Core Team. R: A language and environment for statistical computing. R Foundation for Statistical Computing, Vienna, Austria. URL https://www.R-project.org/(2021).
71. Love, M. I., Huber, W., Anders, S. (2014). Moderated estimation of fold change and dispersion for RNA-seq data with DESeq2. Genome Biology 15, 550.
72. Zhang X., Smits A. H., van Tilburg G. B. A., Ovaa H., Huber W. & Vermeulen M. (2018). Proteome-wide identification of ubiquitin interactions using UbIA-MS. Nat Protoc 13, 530-550.
73. Gatto L., Lilley K. S. (2012). MSnbase-an R/Bioconductor package for isobaric tagged mass spectrometry data visualization, processing, and quantitation. Bioinformatics 28, 288-289.
74. Ritchie M. E., Phipson B., Wu D., Hu Y., Law C. W., Shi W., Smyth G. K. (2015). limma powers differential expression analyses for RNA-sequencing and microarray studies. Nucleic Acids Research 43, e47.
75. Mellacheruvu D., Wright Z., Couzens A. L., et al. (2013). The CRAPome: a Contaminant Repository for Affinity Purification Mass Spectrometry Data. Nature Methods 10, 730-736.
76. Orlando D. A., Chen M. W., Brown V. E., Solanki S., Choi Y. J., Olson E. R., Fritz C. C., Bradner J. E., Guenther M. G. (2014). Quantitative ChIP-Seq normalization reveals global modulation of the epigenome. Cell Rep. 9, 1163-70.
77. Heng L. and Durbin R. (2009). Fast and accurate short read alignment with Burrows-Wheeler transform. Bioinformatics 25, 1754-1760.
78. Ross-Innes, C. S., Stark R., Teschendorff A. E., Holmes K. A., Ali H. R., Dunning M. J., Brown G. D., Gojis O., Ellis I. O., Green A. R., Ali S., Chin S., Palmieri C., Caldas C. & Carroll J. S. (2012). Differential oestrogen receptor binding is associated with clinical outcome in breast cancer. Nature 481, 389-393.
79. Zhu J. L., Zhu L. J., Gazin C., Lawson N. D., Pages H., Lin S. M., Lapointe D. S. & Green M. R. (2010). ChIPpeakAnno: a Bioconductor package to annotate ChIP-seq and ChIP-chip data. BMC Bioinformatics 11, 237.
80. Ritz C., Baty F., Streibig J. C., Gerhard D. (2015). Dose-Response Analysis Using R. Plos One 10, e0146021.

### Example 21. Leveranin2 β-Catenin-Ikaros Complexes in Autoimmune Diseases

β-catenin drives transcriptional activation of MYC in any other cell types. In B-cells, it was discovered herein, that β-catenin pairs with Ikaros factors for repression of MYC. Pathologically activated B-cells in systemic lupus erythematosus (SLE) and rheumatoid arthritis (RA) are uniquely dependent on GSK3β-mediated degradation of β-catenin. Hence, it was proposed herein to leverage GSK3β-inhibition as new strategy to mitigate acute flares of SLE and RA.

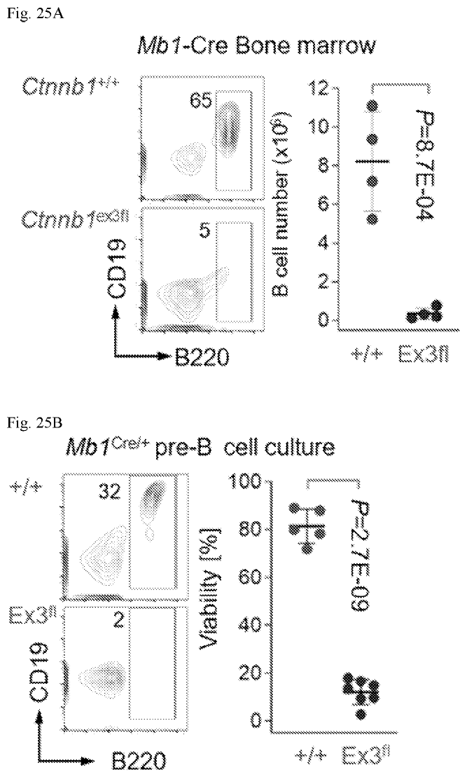

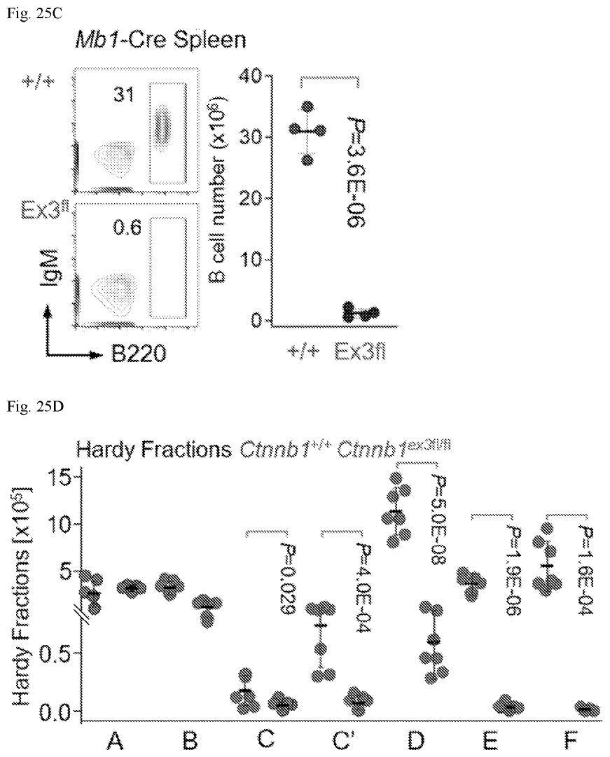

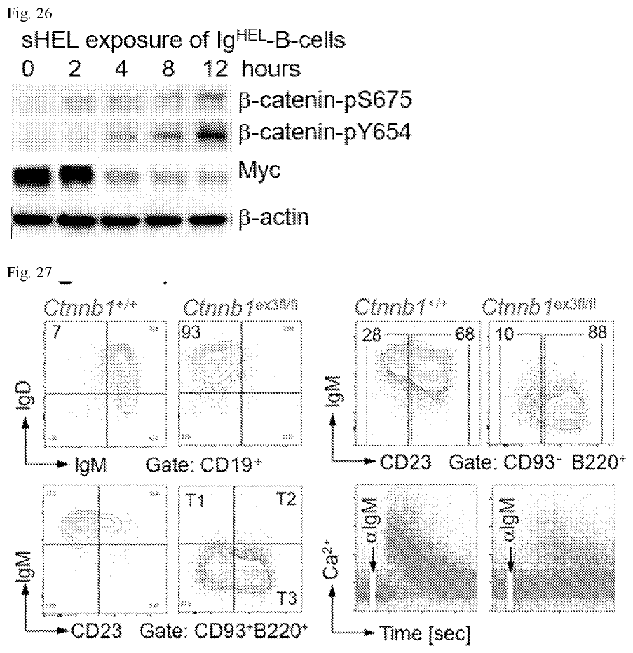

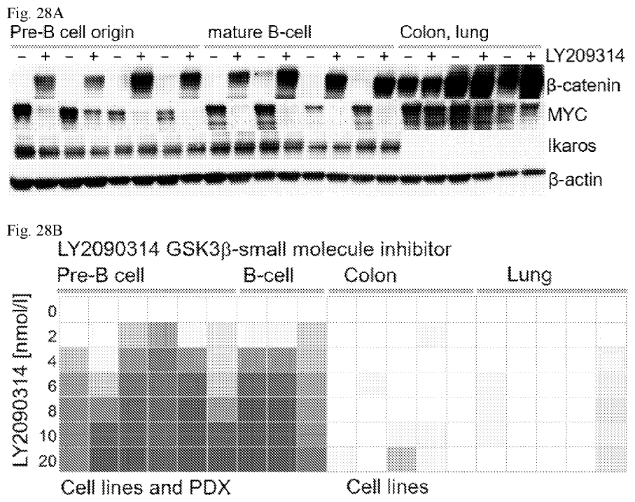

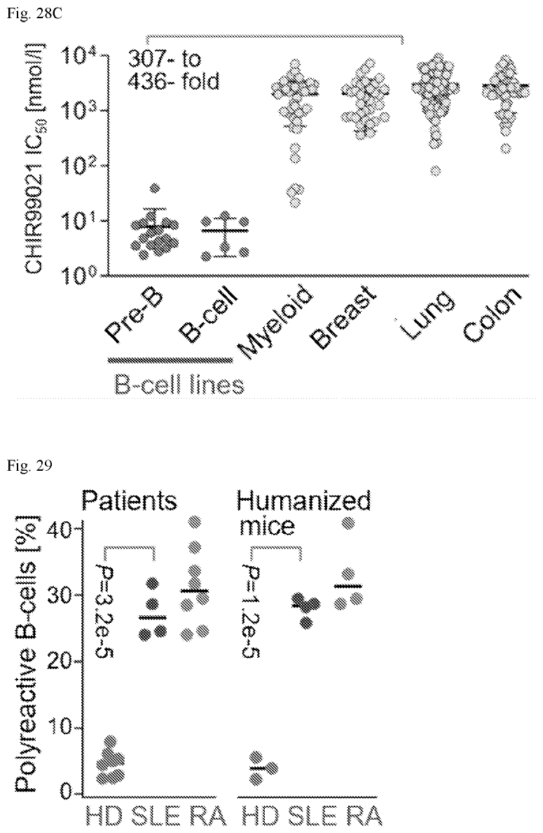

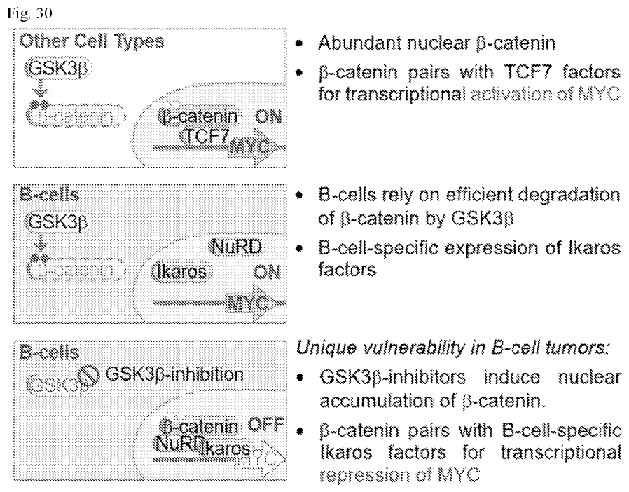

In most cell types, β-catenin promotes transcriptional activation of MYC¹⁻³ and is essential for proliferation and survival. Previous studies showed that β-catenin is dispensable for B-cell development⁴. In contrast to other cell types, it was discovered herein that B-cells consistently lack expression of β-catenin (FIG. 1) and critically depend on GSK3β-dependent phosphorylation of serine residues in exon 3 of β-catenin, to initiate and β-catenin degradation⁵⁻⁶. Cre-mediated excision of GSK3β-phosphorylation sites induced β-catenin accumulation and near complete loss of B-lymphopoiesis beyond the pre-B cell stage (FIG. 25). While genetic deletion of β-catenin enabled clonal expansion of autoreactive B-cells, inhibition of GSK3β induced nuclear accumulation of β-catenin, anergy and cell death (FIGS. 27-28). It was discovered herein that pathological signaling in autoreactive B-cells induced aberrant nuclear accumulation of β-catenin (FIG. 26). For these reasons, it was hypothesized that nuclear β-catenin accumulation functions as a sensor of pathological signaling to eliminate autoreactive B-cells, which can be leveraged for the treatment of autoimmune diseases by GSK3β small molecule inhibitors.

In epithelial cells, β-catenin forms complexes with TCF7 family factors for transcriptional activation of MYC⁷. Instead of TCF7, the present interactome studies in activated B-cells revealed that, β-catenin formed complexes with lymphoid Ikaros zinc finger (IKZF) transcription factors (FIG. 4) for transcriptional repression of MYC at a recently discovered MYC ‘blood enhancer cluster’ (BENC; FIG. 6)⁹. The central hypothesis that activation of β-catenin engages repressive complexes with Ikaros factors to regulate B-cell selection through MYC-repression was tested.

Glucocorticoid paradigm: β-catenin accumulation as selective vulnerability of pathological B-cells. For the past 60 years, glucocorticoids¹⁰ have been central empirical components of nearly all treatment regimens for lymphoproliferative and autoimmune diseases. Glucocorticoids selectively suppress pathogenic clones in active flares of autoimmune disease but have no effect on activated myeloid cells and solid tumors¹⁰. In analogy to the glucocorticoid paradigm, it was assessed herein the concept that targeted accumulation of β-catenin represents a selective vulnerability of pathological B-cell clones in active flares of autoimmune diseases. It was discovered herein that small molecule GSK30 inhibitors induce nuclear β-catenin accumulation to form β-catenin-Ikaros complexes for targeted repression of MYC. Mechanistic experiments demonstrated that this approach was highly effective in eliminating pathological B-cell clones in active flares of autoimmune disease. Four GSK3β-inhibitors underwent full clinical development and demonstrated favorable safety profiles in phase 2 trials for neurologic disorders and solid tumors (Table 11). GSK3P-inhibitors were evaluated for their ability to engage repressive β-catenin-Ikaros complexes to eliminate pathogenic clones in autoimmune diseases.

The following aspects, but not limited to, contribute innovative concepts to the understanding of how β-catenin-Ikaros complexes regulate B-cell selection and prevent autoimmune diseases: i) discovery that β-catenin forms repressive complexes with Ikaros-factors, which is in contrast to activating β-catenin:TCF7 complexes in other cell lineages (FIG. 4); ii) innovative concept that lymphoid Ikaros-factors coopt β-catenin to recruit repressive nucleosome remodeling and deacetylase (NuRD) complex components (MTA1, MTA2, CHD4, GATAD2A, GATAD2B) for transcriptional repression of β-catenin targets (FIGS. 4, 6); iii) identification of MYC ‘blood enhancer cluster’ (BENC)9 as central target of β-catenin-Ikaros (FIG. 6) iv) discovery of GSK3β inhibitors to engage β-catenin-Ikaros complexes as a strategy to selectively kill pathological B-cells in autoimmune diseases (FIGS. 28-29); v) using ssDNA HDRT-mediated genetic editing”, to engineer GFP-tagged knockin alleles carrying point mutations of Ikaros-motifs in MYC BENC enhancer regions and determine how these mutations affect B-cell autoimmunity in humanized mice (FIG. 29); vi) using dCas9-APEX2-based genomic locus proteomics (GLoPro) proximity labeling and proteomics², to comprehensively identify components of β-catenin-Ikaros complexes binding the BENC region (FIG. 6).

It was found herein that activated B-cells were highly sensitive to nuclear β-catenin-accumulation and critically depend on its negative regulation by GSK30. Inhibition of GSK3β induced nuclear accumulation of β-catenin, formation of complexes with Ikaros, repression of MYC and ultimately anergy and cell death (FIG. 6). Conversely, genetic deletion or downregulation of β-catenin enabled autoreactive B-cells to evade negative selection¹³⁻¹⁹. In analogy to the glucocorticoid-paradigm, the central hypothesis that GSK30 inhibition represents a powerful approach to selectively eliminate pathological B-cell clones in SLE and RA is assessed.

### Targeted Engagement of β-Catenin-Ikaros Complexes in Genetic Mouse Models for RA and SLE.

Previous studies suggested that GSK3β inhibitors can curb active flares in mouse models for autoimmune diseases including rheumatoid arthritis (RA)²⁰ and multiple sclerosis (EAE)²¹, although the mechanism was unknown. Here the hypothesis is tested that small molecule inhibitors of GSK3β are useful to engage suppressive β-catenin-Ikaros complexes in autoreactive B-cells to restore tolerance mechanisms and eradicate autoreactive clones in genetic mouse models for autoimmune diseases including SLE and RA. Consistent with a function as sensor for pathological B-cell signaling, inducible accumulation of β-catenin in splenic mature B-cells caused anergic phenotypes (FIG. 27) reminiscent of anergic Ig^{HEL} B-cells when exposed to soluble HEL²² (FIG. 26).

β-catenin activation in mouse models for SLE. As mouse models for SLE, lupus-prone B6.Sle1.Yaa mice will be studied, to be crossed on the B6Mb1^{+/Cre}x Ctnnb1^{ex3fl/fl} model for B-cell-specific accumulation of β-catenin (FIG. 25). B6.Sle1.Yaa mice only require one backcross then intercross for Sle1 homozygosity, a large locus on chromosome 1 that promotes lupus susceptibility. One caveat of the B6.Sle1.Yaa model is that these are male mice, by contrast to female-dominant lupus in patients, since disease penetrance requires a second copy of Tlr7 on the Y chromosome (the Yaa allele). The B6.Sle1.Yaa faithfully replicates these outcomes as observed in other lupus-prone strains, including female predominant strains such as NZB/WF₁ and MRL/1pr, or single gene models.

β-catenin activation in mouse models for RA. For collagen-induced arthritis (CIA) as a model for RA, disease is modeled on a B6 background with the B6 Mbl^{+/Cre}×Ctnnb1^{ex3fl/fl} allele using chicken type II collagen as an immunogen. Compared to DBA1 mice, the phenotype is somewhat milder but does not require backcrosses. Both models of inflammatory synovitis have been established, CIA induced in DBA1 and B6. The colonies are readily available for analyzing outcomes in these animals, including detailed analysis of B- and T-cell responses, and degree of synovitis and joint damage.

To study if β-catenin-Ikaros complexes can delay or prevent the onset of SLE and RA, these models are studied on a B6 Mbl^{+/Cre}×Ctnnb1^{fl/f1} background. To test whether β-catenin-Ikaros complexes can alleviate already established SLE and RA, small molecule GSK3β-inhibition is leveraged for β-catenin-Ikaros-mediated suppression of Myc in pathogenic B-cell populations. Once full-blown disease has developed the effect of selective pharmacological GSK3β-inhibition is tested by injection of LY2090314 (4 injections i.p., 20 mg/kg). If pharmacological engagement of β-catenin-Ikaros complexes is sufficient to kill autoreactive B-cells in an active flare of SLE and RA, repeated injection of LY2090314 is expected to induce disease remission.

### Preclinical Evaluation of GSK3fl-Inhibitors in Humanized Mouse Models for RA and SLE.

Humanized mice engrafted with HSCs isolated from the bone marrow of four patients with SLE and four patients with RA were generated (FIG. 29). Mice engrafted with patients' HSCs generated elevated frequencies of autoreactive B-cells compared to mice engrafted with HSCs from health donors, similar to those in the blood of patients and healthy donors (FIG. 29). It is proposed herein to determine BCR-reactivity in flow-sorted human CD19⁺ CD10⁺ IgM⁺ IgD^{lo/-} immature B-cells and peripheral extrafollicular autoreactive CD19^{hi} CD21^{lo/-} CD27⁻ IgD⁻ double negative (DN2) B-cells isolated from the bone marrow and blood of eight patients with SLE and eight patients with RA, respectively²³⁻²⁴. Three longitudinal samples are be studied for each patient, (i) at the time of steady state (asymptomatic), (ii) active flare and (iii) post-remission.

Power calculations for patient numbers are based on previous studies of SLE and RA (FIG. 29). Hence, the proposed sample size of 8 SLE and 8 RA patients would yield >0.95 power to detect a significant difference from healthy donors with an α of 0.05 by nonparametric Mann-Whitney U tests. It is unlikely that differences linked to sex, age, and other biological variables can be assessed, however, this study can generate preliminary data for future analyses of larger patient cohorts beyond this study.

This study is based on the discovery that pathological B-cells in acute flares of autoimmune diseases are uniquely dependent on negative regulation of β-catenin by GSK30. Four FDA-approved GSK3β inhibitors have undergone full clinical development. In 18 clinical trials for patients with neurological conditions and solid tumors (Table 11), none of the four GSK3β-small molecule inhibitors achieved clinical responses but were well tolerated with favorable safety and PK/PD profiles. It is anticipated that the repurposing efforts of GSK3β-inhibitors for SLE and RA will benefit from existing safety profiles (DLT, MTD), PK/PD and toxicology measurements.

TABLE 11
Clinical trials of four approved GSK3β-small molecule inhibitors

| Small | | | |
|---|---|---|---|
| Molecule | Indication | NCT IDs | Outcome |
| LY2090314 | Metastatic cancer, | NCT01287520, NCT01214603 | Phase 1 and 2, favorable safety |
| | pancreatic cancer | NCT01632306 | No clinical responses, MTD 80 mg i.v |
| Tideglusib | Myotonic dystrophy, | NCT01350362, NCT00948259 | Phase 1 and 2, favorable safety |
| | Parkinson, | | No clinical responses, MTD 120 mg i.v. |
| | Alzheimer's | | |
| 9-ING-4 | Pancreatic cancer, | NCT03678883, NCT05239182, NCT04239092 | Phase 1 and 2, favorable safety |
| | Advanced Sarcomas | NCT05077800, NCT04906876, NCT03678883 | No clinical response |
| | | NCT05116800, NCT04218071, NCT04832438 | |
| CHIR99021 | Ovarian cancer, | NCT03081780, NCT03213964 | Phase 1 and 2, favorable safety |
| | Advances solid tumors | NCT03319459 | No clinical responses, MTD 80 mg i.v |

### REFERENCES

1. V. Brault, R. Moore, S. Kutsch, M. Ishibashi, D. H. Rowitch, A. P. McMahon, L. Sommer, O. Boussadia, R. Kemler. Inactivation of the beta-catenin gene by Wntl-Cre-mediated deletion results in dramatic brain malformation and failure of craniofacial development. Development 128, 1253-64 (2001).
2. J. Huelsken, R. Vogel, B. Erdmann, G. Cotsarelis, W. Birchmeier. Beta-catenin controls hair follicle morphogenesis and stem cell differentiation in the skin. Cell 105, 533-45 (2001).
3. van de Wetering M, Sancho E, Verweij C, et al. The beta-catenin/TCF-4 complex imposes a crypt progenitor phenotype on colorectal cancer cells. Cell 111, 241-250 (2002).
4. M. Cobas, A. Wilson, B. Ernst, S. J. Mancini, H. R. MacDonald, R. Kemler, F. Radtke. Beta-catenin is dispensable for hematopoiesis and lymphopoiesis. J. Exp. Med. 199, 221-9 (2004).
5. C. Liu, Y. Li, M. Semenov, C. Han, G. H. Baeg, Y. Tan, Z. Zhang, X. Lin, X. He. Control of beta-catenin phosphorylation/degradation by a dual-kinase mechanism. Cell, 108, 837-47 (2002).
6. J. Behrens, B. A. Jerchow, M. Würtele, J. Grimm, C. Asbrand, R. Wirtz, M. K0 hl, D. Wedlich, W. Birchmeier. Functional interaction of an axin homolog, conductin, with beta-catenin, APC, and GSK3beta. Science 280, 596-9 (1998).
7. T. C. He, A. B. Sparks, C. Rago, H. Hermeking, L. Zawel, L. T. da Costa, P. J. Morin, B. Vogelstein, K. W. Kinzler. Identification of c-MYC as a target of the APC pathway. Science, 281, 1509-12 (1998).
8. Bienz M. β-catenin: a pivot between cell adhesion and Wnt signalling. Curr Biol. 15: 64-67 (2005).
9. Bahr C, von Paleske L, Uslu V V, Remeseiro S, Takayama N, Ng S W, Murison A, Langenfeld K, Petretich M, Scognamiglio R, Zeisberger P, Benk A S, Amit I, Zandstra P W, Lupien M, Dick J E, Trumpp A, Spitz F. A Myc enhancer cluster regulates normal and leukaemic haematopoietic stem cell hierarchies. Nature. 553: 515-520 (2018).
10. Pearson O H, Eliel L P, et al. Adrenocorticotropic hormone- and cortisone-induced regression of lymphoid tumors in man; a preliminary report. Cancer 2, 943-945 (1949)
11. Shy B R, Vykunta V S, Ha A, Talbot A, Roth T L, Nguyen D N, Pfeifer W G, Chen Y Y, Blaeschke F, Shifrut E, Vedova S, Mamedov M R, Chung J J, Li H, Yu R, Wu D, Wolf J, Martin T G, Castro C E, Ye L, Esensten J H, Eyquem J, Marson A. High-yield genome engineering in primary cells using a hybrid ssDNA repair template and small-molecule cocktails. Nat Biotechnol. 18: 9003 (2022).
12. Myers S A, Wright J, Peckner R, Kalish B T, Zhang F, Carr S A. Discovery of proteins associated with a predefined genomic locus via dCas9-APEX-mediated proximity labeling. Nat Methods. 15: 437-439 (2018).
13. Tan K, Xie X, Shi W, Miao L, Dong X, Yang W, Shao C, Zhao H, Wang Y, Wang G, Hou F, Hong Y. Deficiency of canonical Wnt/β-catenin signalling in hepatic dendritic cells triggers autoimmune hepatitis. Liver Int. 40:131-140 (2020).
14. Orme JJ, Du Y, Vanarsa K, Wu T, Satterthwaite AB, Mohan C. Leukocyte Beta-Catenin Expression Is Disturbed in Systemic Lupus Erythematosus. PLoS One. 11: e0161682 (2016).
15. Lengfeld J E, Lutz S E, Smith J R, Diaconu C, Scott C, Kofman S B, Choi C, Walsh C M, Raine C S, Agalliu I, Agalliu D. Endothelial Wnt/β-catenin signaling reduces immune cell infiltration in multiple sclerosis. Proc Natl Acad Sci USA. 114: 1168-1177 (2017).
16. Manicassamy S, Reizis B, Ravindran R, Nakaya H, Salazar-Gonzalez R M, Wang Y C, Pulendran B. Activation of beta-catenin in dendritic cells regulates immunity versus tolerance in the intestine. Science 329: 849-853 (2010).
17. Wu F, Mao C, Mou X, Xu C, Zheng T, Bu L, Luo X, Lu Q, Wang X. Decreased β-catenin expression contributes to IFNγ-induced chemokine secretion and lymphocyte infiltration in Hashimoto's thyroiditis. Endocr Connect. 11: e210451 (2022).
18. Sumida T, Lincoln M R, Ukeje C M, Rodriguez D M, Akazawa H, Noda T, Naito A T, Komuro I, Dominguez-Villar M, Hafler D A. Activated β-catenin in Foxp3+ regulatory T cells links inflammatory environments to autoimmunity. Nat Immunol. 19: 1391-1402 (2018).
19. Mastrogiovanni M, Vargas P, Rose T, Cuche C, Esposito E, Juzans M, Laude H, Schneider A, Bernard M, Goyard S, Renaudat C, Ungeheuer M N, Delon J, Alcover A, Di Bartolo V. The tumor suppressor adenomatous polyposis coli regulates T lymphocyte migration. Sci Adv. 8: eab15942 (2022).
20. Kwon Y J, Yoon C H, Lee S W, Park Y B, Lee S K, Park M C. Inhibition of glycogen synthase kinase-3β suppresses inflammatory responses in rheumatoid arthritis fibroblast-like synoviocytes and collagen-induced arthritis. Joint Bone Spine. 81: 240-246 (2014).
21. Beurel E, Kaidanovich-Beilin O, Yeh W I, Song L, Palomo V, Michalek S M, Woodgett J R, Harrington L E, Eldar-Finkelman H, Martinez A, Jope R S. Regulation of Th1 cells and experimental autoimmune encephalomyelitis by glycogen synthase kinase-3. J Immunol. 190: 5000-5011 (2013).
22. Goodnow C C, Crosbie J, Adelstein S, Lavoie T B, Smith-Gill S J, Brink R A, Pritchard-Briscoe H, Wotherspoon J S, Loblay R H, Raphael K, Trent R J, Basten A. Altered immunoglobulin expression and functional silencing of self-reactive B lymphocytes in transgenic mice. Nature. 334: 676-82 (1988).
23. Sadras T, Martin M, Kume K, Robinson M E, Saravanakumar S, Lenz G, Chen Z, Song SY, Siddiqi T, Oksa L, Knapp A M, Cutler J, Cosgun K N, Klemm L, Ecker V, Kiefer F, Heisterkamp N, Pandey A, Wang L, Jumaa H, Buchner M, Ruland J, Chan W C, Meffre E, Martin T & Muschen M. Developmental partitioning of SYK and ZAP70 prevents autoimmunity and cancer. Mol Cell 81: 644-654 (2021)
24. Schickel J N, Kuhny M, Baldo A, Bannock J M, Massad C, Wang H, Katz N, Oe T, Menard L, Soulas-Sprauel P, Strowig T, Flavell R, Meffre E. PTPN22 inhibition resets defective human central B cell tolerance. Sci Immunol. 1:aaf7153 (2016).

1. A method of treating a lymphocyte associated disease or condition, the method comprising administering to a subject in need thereof an effective amount of an agonist or activator of a β-catenin:Ikaros zinc finger (IKZF) protein complex.

2. The method of claim 1, wherein the agonist or activator of the β-catenin:IKZF protein complex is an agent that inhibits the expression or function of Glycogen Synthase Kinase 33 (GSK303), Axis Inhibition Protein 1 (AXIN1), Axis Inhibition Protein 2 (AXIN2), Adenomatous Polyposis Coli (APC), and/or beta-transducin repeat containing (beta-TCRP).

3. The method of claim 2, wherein the agonist or activator of the β-catenin:IKZF protein complex is a small molecule, a peptide, an antibody or antibody fragment, an siRNA, an shRNA, a gapmer, an antisense oligonucleotide, an aptamer, PROTAC-degrader, or a site-specific nuclease.

4. The method of claim 2, wherein the agent that inhibits the expression or function of GSK3β3 is a GSK3β3 inhibitor.

5. The method of claim 4, wherein the GSK3β inhibitor is a small molecule, a peptide, an antibody or antibody fragment, an siRNA, an shRNA, a gapmer, an antisense oligonucleotide, an aptamer, PROTAC-degrader, or a site-specific nuclease.

6. The method of claim 5, wherein the small molecule GSK3β inhibitor is a diazepinoindole, a biindole, an aminopyrimidine, a thiadiazolidine, or a maleimide-based molecule.

7. The method of claim 6, wherein the diazepinoindole is LY2090314.

8. The method of claim 6, wherein the biindole is 6-Bromoindirubin-3′-oxime.

9. The method of claim 6, wherein the aminopyrimidine is CHIR98014 or CHIR99021.

10. The method of claim 6, wherein the thiadiazolidine is Tideglusib.

11. The method of claim 6, wherein the maleimide-based molecule is 9-ING-41.

12. The method of any one of claims 4-11, wherein the GSK3 β inhibitor is administered to the subject a dose sufficient to result in a steady state plasma concentration of the inhibitor from about 5 nM to about 500 nM.

13. The method of any one of claims 4-11, wherein the inhibitor inhibits GSK3β with an IC₅₀ of 100 nM or less.

14. The method of claim 3 or 5, wherein the site-specific nuclease is an engineered homing endo-nuclease or meganuclease, a zinc-finger nucleases (ZFNs), a transcription activator-like effector nucleases (TALENs), or a clustered regularly interspaced short palindromic repeat (CRISPR) system.

15. The method of any one of claims 1-14, wherein the IKZF protein is IKZF1, IKZF2, or IKZF3.

16. The method of claim 15, wherein the IKZF protein is IKZF1 or IKZF3.

17. The method of any one of claims 1-16, wherein the lymphocyte associated disease or condition is a B-lymphoid malignancy, a T-lymphoid malignancy, or a combination of both.

18. The method of any one of claims 1-16, wherein the lymphocyte associated disease or condition is a premalignant condition or a cancer.

19. The method of claim 18, wherein the premalignant condition is lymphoid clonal hematopoiesis of indeterminate potential (L-CHIP), Monoclonal B lymphocytosis (MBL), or a monoclonal gammopathy of unknown significance (MGUS).

20. The method of claim 18, wherein the cancer is a metastatic cancer.

21. The method of claim 18, wherein the cancer is an acute T-lymphoblastic lymphoma/leukemia (T-ALL), a peripheral T-cell lymphoma (PTCL), a cutaneous T-cell lymphomas, an adult T-cell leukemia/lymphoma, an angioimmunoblastic T-cell lymphoma, an extranodal natural killer/T-cell lymphoma, an enteropathy-associated intestinal T-cell lymphoma (EATL), an anaplastic large cell lymphoma (ALCL), a peripheral T-cell lymphoma not otherwise specified cancer (PTCL-NOS), a B-cell acute lymphoblastic leukemia (B-ALL), a diffuse large B-cell lymphoma (DLBCL), a follicular lymphoma, a chronic lymphocytic leukemia (CLL)/small lymphocytic lymphoma (SLL), a mantle cell lymphoma (MCL), a marginal zone lymphoma, a Burkitt lymphoma, a lymphoplasmacytic lymphoma (Waldenstrom macroglobulinemia), a hairy cell leukemia, a primary central nervous system (CNS) lymphoma, a primary intraocular lymphoma, or a non-Hodgkin lymphoma (NHL).

22. The method of any one of claims 1-16, wherein the lymphocyte associated disease or condition is an autoimmune disease.

23. The method of claim 22, wherein the autoimmune disease or condition is rheumatoid arthritis, systemic lupus erythematosus, vasculitis, scleroderma, or Sjogren disease.

24. The method of any one of claims 1-16, wherein the lymphocyte associated disease or condition is a graft versus host disease (GvHD).

25. The method of any one of claims 1-23, wherein the inhibitor of the β-catenin:IKZF protein complex is administered in combination with at least one other treatment regimen for the lymphocyte associated disease or condition.

26. The method of claim 25, wherein the at one least other treatment comprises glucocorticoids; azathioprine; methotrexate; a combination of vincristine, prednisolone, L-asparaginase, daunorubicin (VPLD); a combination of cyclophosphamide, vincristine, Adriamycin, and dexamethasone (hyper-CVAD); a combination of rituximab, cyclophosphamide, doxorubicin, vincristine, and prednisone (R-CHOP); a combination of cyclophosphamide, doxorubicin, vincristine, and prednisone (CHOP); or combinations thereof.

27. The method of any one of claims 1-26, wherein the disease or condition is a drug-resistant disease or condition.

28. The method of any one of claims 1-27, wherein the agonist or activator is administered intravenously, subcutaneously, or orally.

29. The method of any one of claims 1-28, wherein the agonist or activator is administered in a dosage range from 5 nM to 100 nM.

30. A method of eradicating pathogenic lymphocyte populations, the method comprising administering to a subject in need thereof a therapeutically effective amount of an agonist or activator of a β-catenin:Ikaros zinc finger (IKZF) protein complex.

31. The method of claim 30, wherein the agonist or activator of the β-catenin:IKZF protein complex is an agent that inhibits the expression or function of Glycogen Synthase Kinase 3β (GSK3β), Axis Inhibition Protein 1 (AXIN1), Axis Inhibition Protein 2 (AXIN2), Adenomatous Polyposis Coli (APC), and/or beta-transducin repeat containing (beta-TCRP).

32. The method of claim 31, wherein the agonist or activator of the β-catenin:IKZF protein complex is a small molecule, a peptide, an antibody or antibody fragment, an siRNA, an shRNA, a gapmer, an antisense oligonucleotide, an aptamer, PROTAC-degrader, or a site-specific nuclease.

33. The method of claim 31, wherein the agent that inhibits the expression or function of GSK3β3 is a GSK3β3 inhibitor.

34. The method of claim 33, wherein the GSK3β inhibitor is a small molecule, a peptide, an antibody or antibody fragment, an siRNA, an shRNA, a gapmer, an antisense oligonucleotide, an aptamer, PROTAC-degrader, or a site-specific nuclease.

35. The method of claim 34, wherein the small molecule GSK3β inhibitor is a diazepinoindole, a biindole, an aminopyrimidine, a thiadiazolidine or a maleimide-based molecule.

36. The method of claim 35, wherein the diazepinoindole is LY2090314.

37. The method of claim 35, wherein the biindole is 6-Bromoindirubin-3′-oxime.

38. The method of claim 35, wherein the aminopyrimidine is CHIR98014 or CHIR99021.

39. The method of claim 35, wherein the thiadiazolidine is Tideglusib.

40. The method of claim 35, wherein the maleimide-based molecule is 9-ING-41.

41. The method of any one of claims 33-40, wherein the GSK3β3 inhibitor is administered to the subject a dose sufficient to result in a steady state plasma concentration of the inhibitor from about 5 nM to about 500 nM.

42. The method of any one of claims 33-40, wherein the inhibitor inhibits GSK3β with an IC₅₀ of 100 nM or less.

43. The method of claim 32 or 34, wherein the site-specific nuclease is an engineered homing endo-nuclease or meganuclease, a zinc-finger nucleases (ZFNs), a transcription activator-like effector nucleases (TALENs), or a clustered regularly interspaced short palindromic repeat (CRISPR) system.

44. The method of any one of claims 30-43, wherein the IKZF protein is IKZF1, IKZF2, or IKZF3.

45. The method of claim 44, wherein the IKZF protein is IKZF1 or IKZF3

46. The method of any of claim 30-45, wherein the pathogenic lymphocyte is a B-lymphocyte, a T-lymphocyte, or a combination of both.

47. The method of any one of claims 30-46, wherein the pathogenic lymphocyte is a drug-resistant pathogenic lymphocyte.

48. The method of any one of claims 30-47, wherein the inhibitor is administered intravenously, subcutaneously, or orally.

49. The method of any one of claims 30-48, wherein the inhibitor is administered in a dosage range from 5 nM to 500 nM.

50. A method of enhancing adoptive cellular therapy (ACT) in a subject, the method comprising administering to a subject in need thereof a therapeutically effective amount of an agonist or activator of a β-catenin:Ikaros zinc finger (IKZF) protein complex.

51. The method of claim 50, wherein the agonist or activator of a β-catenin:IKZF protein complex is administered to the subject before administration of the ACT.

52. The method of claim 50 or claim 51, wherein the agonist or activator of the (β-catenin:IKZF protein complex is an agent that inhibits the expression or function of Glycogen Synthase Kinase 3β (GSK3β), Axis Inhibition Protein 1 (AXIN1), Axis Inhibition Protein 2 (AXIN2), Adenomatous Polyposis Coli (APC), and/or beta-transducin repeat containing (beta-TCRP).

53. The method of claim 52, wherein the agonist or activator of the β-catenin:IKZF protein complex is a small molecule, a peptide, an antibody or antibody fragment, an siRNA, an shRNA, a gapmer, an antisense oligonucleotide, an aptamer, PROTAC-degrader, or a site-specific nuclease.

54. The method of claim 52, wherein the agent that inhibits the expression or function of GSK3β is a GSK3β inhibitor.

55. The method of claim 54, wherein the GSK3β inhibitor is a small molecule, a peptide, an antibody or antibody fragment, an siRNA, an shRNA, a gapmer, an antisense oligonucleotide, an aptamer, PROTAC-degrader, or a site-specific nuclease.

56. The method of claim 55, wherein the small molecule GSK3β inhibitor is a diazepinoindole, a biindole, an aminopyrimidine, a thiadiazolidine, or a maleimide-based molecule.

57. The method of claim 56, wherein the diazepinoindole is LY2090314.

58. The method of claim 56, wherein the biindole is 6-Bromoindirubin-3′-oxime.

59. The method of claim 56, wherein the aminopyrimidine is CHIR98014 or CHIR99021.

60. The method of claim 56, wherein the thiadiazolidine is Tideglusib.

61. The method of claim 56, wherein the maleimide-based molecule is 9-ING-41.

62. The method of any one of claims 54 to 61, wherein the GSK3 β inhibitor is administered to the subject a dose sufficient to result in a steady state plasma concentration of the inhibitor from about 5 nM to about 500 nM.

63. The method of any one of claims 54 to 61, wherein the inhibitor inhibits GSK3β with an IC₅₀ of 100 nM or less.

64. The method of claim 53 or 55, site-specific nuclease is an engineered homing endo-nuclease or meganuclease, a zinc-finger nucleases (ZFNs), a transcription activator-like effector nucleases (TALENs), or a clustered regularly interspaced short palindromic repeat (CRISPR) system.

65. The method of any one of claims 50-64, wherein the IKZF protein is IKZF1, IKZF2, or IKZF3.

66. The method of claim 65, wherein the IKZF protein is IKZF1 or IKZF3.

67. The method of any one of claims 50-66, wherein the inhibitor is administered intravenously, subcutaneously, or orally.

68. The method of any one of claims 50-67, wherein the inhibitor is administered in a dosage range from 5 nM to 500 nM.

69. A method of treating a lymphopenic associated disease or condition, the method comprising administering to a subject in need thereof an effective amount of an agent that inhibits the expression or function of β-catenin or a β-catenin:Ikaros zinc finger (IKZF) protein complex.

70. The method of claim 69, wherein the agent that inhibits the expression or function of β-catenin inhibitor or a β-catenin:Ikaros zinc finger (IKZF) protein complex is a small molecule, a peptide, an antibody or antibody fragment, an siRNA, an shRNA, a gapmer, an antisense oligonucleotide, an aptamer, PROTAC-degrader, or a site-specific nuclease.

71. The method of claim 70, wherein the site-specific nuclease is an engineered homing endo-nuclease or meganuclease, a zinc-finger nucleases (ZFNs), a transcription activator-like effector nucleases (TALENs), or a clustered regularly interspaced short palindromic repeat (CRISPR) system.

72. The method of any one of claims 69-71, wherein the β-catenin gene is knocked out or knocked down.

73. The method of any one of claims 69-72, wherein the lymphopenic associated disease or condition is lymphocytopenia and/or bone marrow failure.

74. The method of any one of claims 69-72, wherein the lymphopenic associated disease or condition is caused by myeloid skewing, immunosenescence, side effects of drug-treatment, bone marrow transplantation, viral infections, and/or immunodeficiencies.

75. A method of enhancing adoptive cellular therapy (ACT) in a subject, the method comprising administering to a subject in need thereof or an ACT preparation a therapeutically effective amount of an agent that inhibits the expression or function of β-catenin or a β-catenin:Ikaros zinc finger (IKZF) protein complex.

76. The method of claim 75, wherein the agent that inhibits the expression or function of β-catenin inhibitor or a β-catenin:Ikaros zinc finger (IKZF) protein complex is a small molecule, a peptide, an antibody or antibody fragment, an siRNA, an shRNA, a gapmer, an antisense oligonucleotide, an aptamer, PROTAC-degrader, or a site-specific nuclease.

77. The method of claim 76, site-specific nuclease is an engineered homing endo-nuclease or meganuclease, a zinc-finger nucleases (ZFNs), a transcription activator-like effector nucleases (TALENs), or a clustered regularly interspaced short palindromic repeat (CRISPR) system.

78. The method of any one of claims 75-77, wherein the β-catenin gene is knocked out or knocked down.


SEQUENCE LISTING (22 sequences)

SEQ ID NO: 1 - DNA, 20 bp, synthetic construct
  source 1..20: mol_type = other DNA
gcgaggtattcggctccgcg

SEQ ID NO: 2 - DNA, 20 bp, synthetic construct
  source 1..20: mol_type = other DNA
acaatggcagacaccatctg

SEQ ID NO: 3 - DNA, 20 bp, synthetic construct
  source 1..20: mol_type = other DNA
ctggagtgtcactgactggg

SEQ ID NO: 4 - DNA, 20 bp, synthetic construct
  source 1..20: mol_type = other DNA
attatgaagccggagcccat

SEQ ID NO: 5 - DNA, 20 bp, synthetic construct
  source 1..20: mol_type = other DNA
ggttcttgactaccgtaatt

SEQ ID NO: 6 - DNA, 20 bp, synthetic construct
  source 1..20: mol_type = other DNA
aaggttatgcaaggtcccag

SEQ ID NO: 7 - protein, 9 aa, synthetic construct
LAGLIDADG

SEQ ID NO: 8 - DNA, 20 bp, synthetic construct
  source 1..20: mol_type = other DNA
cgattgctgagaggaactgg

SEQ ID NO: 9 - DNA, 23 bp, synthetic construct
  source 1..23: mol_type = other DNA
agtgtaaagacttggtccacctg

SEQ ID NO: 10 - DNA, 18 bp, synthetic construct
  source 1..18: mol_type = other DNA
caggacagggtggtcacg

SEQ ID NO: 11 - DNA, 20 bp, synthetic construct
  source 1..20: mol_type = other DNA
aactggcttttggtgtcggg

SEQ ID NO: 12 - DNA, 22 bp, synthetic construct
  source 1..22: mol_type = other DNA
tcggtggcttgctgattatttc

SEQ ID NO: 13 - DNA, 21 bp, synthetic construct
  source 1..21: mol_type = other DNA
ttcagccttcagtctaacatc

SEQ ID NO: 14 - DNA, 18 bp, synthetic construct
  source 1..18: mol_type = other DNA
atctgtgaagacagggtg

SEQ ID NO: 15 - DNA, 19 bp, synthetic construct
  source 1..19: mol_type = other DNA
ccctgtggatgccacctcc

SEQ ID NO: 16 - DNA, 19 bp, synthetic construct
  source 1..19: mol_type = other DNA
gtcctggcatctgtcagag

SEQ ID NO: 17 - DNA, 13 bp, synthetic construct
  source 1..13: mol_type = other DNA
caggggaaggagc

SEQ ID NO: 18 - DNA, 13 bp, synthetic construct
  source 1..13: mol_type = other DNA
cagaggaagattg

SEQ ID NO: 19 - DNA, 12 bp, synthetic construct
  source 1..12: mol_type = other DNA
agccagaagtgg

SEQ ID NO: 20 - DNA, 17 bp, synthetic construct
  source 1..17: mol_type = other DNA
cagccaggggaaggagc

SEQ ID NO: 21 - DNA, 17 bp, synthetic construct
  source 1..17: mol_type = other DNA
cagccagaattcggagc

SEQ ID NO: 22 - DNA, 18 bp, synthetic construct
  source 1..18: mol_type = other DNA
cagccagaattcggaagc